(19) United States
(12) Patent Application Publication
(10) Pub. No.: US 2026/0219274 A1
(43) Pub. Date: Jul. 30, 2026

(54) POLYOL SWEETNERS AND CARDIOVASCULAR AND THROMBOTIC EVENT RISK

(71) Applicant: THE CLEVELAND CLINIC FOUNDATION, Cleveland, OH (US)

(72) Inventor: Stanley L. HAZEN, Cleveland, OH (US)

(21) Appl. No.: 19/146,757

(22) PCT Filed: Jan. 9, 2024
(86) PCT No.: PCT/US2024/010901

§ 371 Date: Jul. 9, 2025

Related U.S. Application Data
(60) Provisional application No. 63/479,141, filed on Jan. 9, 2023.

(51) Int. Cl. G01N 33/58; A61K 31/616; G01N 30/72; G01N 30/86; G16H 10/40; G16H 50/30

(52) U.S. Cl. CPC G01N 33/58; A61K 31/616; G01N 30/72; G01N 30/8631; G16H 10/40; G16H 50/30; G01N 2458/15; G01N 2800/2871; G01N 2800/32; G01N 2800/50; G01N 2800/52

(57) ABSTRACT

Provided herein are compositions, systems, kits, and methods for determining the level of a polyol sweetener in a biological sample from a subject (e.g., a subject with, or suspected of having, or at increased risk of developing: a cardiovascular disease, a major adverse cardiovascular event, or enhanced thrombosis). In certain embodiments, determining such polyol sweetener levels are higher than a control level allows a subject to be prescribed, or administered, a CVD therapeutic, anti-thrombotic agent, or lifestyle change to reduce consumption of such polyol sweeteners. In certain embodiments, xylitol is detected in a biological sample from a subject such that xylitol's structural isomers arabitol and rabitol are distinguished. In other embodiments, erythritol is detected in a biological sample from a subject such that erythritol's structural isomer threitol is distinguished.

The present application claims priority to U.S. Provisional application Ser. No. 63/479,141, filed Jan. 9, 2023, which is herein incorporated by reference in its entirety.

### FIELD

Provided herein are compositions, systems, kits, and methods for determining the level of a polyol sweetener in a biological sample from a subject (e.g., a subject with, or suspected of having, or at increased risk of developing: a cardiovascular disease, a major adverse cardiovascular event, or enhanced thrombosis). In certain embodiments, determining such polyol sweetener levels are higher than a control level allows a subject to be prescribed, or administered, a CVD therapeutic, anti-thrombotic agent, or lifestyle change to reduce consumption of such polyol sweeteners. In certain embodiments, xylitol is detected in a biological sample from a subject such that xylitol's structural isomers arabitol and rabitol are distinguished. In other embodiments, erythritol is detected in a biological sample from a subject such that erythritol's structural isomer threitol is distinguished.

### BACKGROUND

In light of the obesity pandemic worldwide(1), measures to reduce sugar intake have become a public health priority. (2) One such measure is the proliferation of artificial sweeteners (including non-nutritive and low-calorie sweeteners (which collectively are also often called “artificial sweeteners”) in processed foods that are promoted as healthy sugar alternatives. Artificial sweeteners are generally regarded as safe and approved by public health authorities (e.g. Food and Drug administration (FDA)(3), European Union (EU)(4). Their use has markedly increased in near universal fashion across all categories, including gender, age, race/ethnicity, weight status, geographic and socio-economic subgroups. Of note, the use of artificial sweeteners is recommended for subjects that suffer from cardiometabolic diseases including obesity, diabetes and cardiovascular disease (CVD), by multiple guideline organizations (e.g. American Heart Association, diabetes associations in the US, UK, Canada and Australia) (5-9) while their long-term cardiovascular adverse effects are rarely studied(10).

Contrary to their intended benefits, artificial sweeteners have been associated with cardiometabolic adverse effects including insulin resistance, type 2 diabetes, incident CVD, atherothrombotic complications and death in a number of epidemiological studies. (11-15). A limited number of randomized controlled trials have examined the short-term effects of sweeteners on metabolic indices with results that in general fail to support the promoted benefits (16-19), though some studies have suggested potential small metabolic improvements. (20,21). The limited randomized controlled trials available on artificial sweeteners have been criticized for not reflecting real-life exposure (22) and for typically being conducted over a short period of time, making them unable to recapitulate long term exposers appropriately for CVD endpoints.

A central limitation of many epidemiological studies and randomized controlled trials that assess health effects of artificial sweeteners is the lack of reliable sweetener quantification. Most clinical studies are based on food questionnaires/dietary recalls that may not adequately reflect artificial sweetener consumption. Further, they often do not differentiate between specific sweeteners, but rather use broad categories (such as low-calorie or artificial sweeteners). Moreover, study subjects likely are not even aware of unintentional consumption because of non-disclosure policies by the FDA.(22). Equally important, artificial sweeteners are not routinely measured because of technical difficulties. In particular, polyol sweeteners have multiple structural isomers that are difficult to separate due to their similar physicochemical properties, and have identical elemental composition and thus molecular weight, making quantification challenging. Thus, while reliable measurement of structurally specific individual sweeteners is needed (to both examine their potential adverse effects and to establish risk thresholds to allow for better informed clinical practice and public health policies), numerous barriers exist.

### SUMMARY

Provided herein are compositions, systems, kits, and methods for determining the level of a polyol sweetener in a biological sample from a subject (e.g., a subject with, or suspected of having, or at increased risk of developing: a cardiovascular disease, a major adverse cardiovascular event, or enhanced thrombosis). In certain embodiments, determining such polyol sweetener levels are higher than a control level allows a subject to be prescribed, or administered, a CVD therapeutic, anti-thrombotic agent, or lifestyle change to reduce consumption of such polyol sweeteners. In certain embodiments, xylitol is detected in a biological sample from a subject such that xylitol's structural isomers arabitol and rabitol are distinguished. In other embodiments, erythritol is detected in a biological sample from a subject such that erythritol's structural isomer threitol is distinguished.

In some embodiments, provided herein are methods of detecting the level of xylitol in a biological sample that may also contain xylitol's structural isomers arabitol and rabitol, comprising: a) mixing a biological sample from a subject with at least one stable isotope labeled structural isomer (SIL isomer) family member selected from: stable isotope labelled xylitol (SIL-xylitol), SIL-arabitol, and SIL-rabitol, b) mixing the biological sample with a hydroxyl group modifying agent such that the SIL isomer and xylitol and any arabitol and rabitol present in said biological sample are modified, thereby generating modified-xylitol and modified SIL isomer, and generating modified-arabitol and modified-rabitol if present; c) subjecting the biological sample to purification such that the modified-xylitol and modified SIL isomer, and modified-arabitol modified-rabitol is present, are substantially isolated from other components of the biological sample, thereby generating a purified sample; d) subjecting said purified sample to mass spectrometry such that peak intensity values are generated for the modified-xylitol and the modified SIL isomer, and the modified-arabitol and modified-rabitol if present; and e) employing the peak intensity value of the modified SIL isomer as an internal standard, and the peak intensity of the modified-xylitol, to determine the level of the xylitol present in the biological sample.

In certain embodiments, the subject has, or is suspected of having, cardiovascular disease, or has an increased risk of: developing cardiovascular disease, having a major adverse cardiovascular event, risk of stroke, or having enhanced thrombosis. In particular embodiments, the biological sample is selected from a urine sample, plasma sample, blood sample, serum sample, sputum, or liquified stool sample. In further embodiments, the hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride. In some embodiment, esterification reactions for polyol sweeteners are performed using the same chemistry as for cyclodextrin (e.g., Blaj et al., Molecules 2023, 28(5), 2001; Garcia, et al., PLoS ONE 2014, 9, e88234; and Li et al., Carbohydr. Res. 2015, 404, 55-62; all three of which are herein incorporated by refence.

In further embodiments, the subject reports a higher than average intake of xylitol in their diet. In some embodiments, the at least one stable isotope labeled structural isomer comprises stable isotope labelled arabitol (SIL-arabitol). In other embodiments, the at least one stable isotope labeled structural isomer comprises stable isotope labelled xylitol (SIL-xylitol). In additional embodiments, the at least one stable isotope labeled structural isomer comprises stable isotope labelled rabitol (SIL-rabitol).

In further embodiments, further comprising generating a report comprising the level of xylitol, wherein the report identifies the level of xylitol as higher than a control level. In additional embodiments, the report describes that the subject should reduce intake of xylitol in order to improve: risk of cardiac disease and/risk of thrombosis. In particular embodiments, the report describes that the subject should be prescribed and/or administered at least one of the following: a CVD therapeutic, an anti-coagulating agent, an anti-platelet agent, a lipid lowering agent, and a blood pressure control agent or therapy. In other embodiments, the mass spectrometry comprises MS/MS, and/or wherein the stable isotope is selected from C13 and deuterium. In particular embodiments, the subject is a human.

In some embodiments, provided herein are methods of detecting the level of erythritol in a biological sample that also contains erythritol's structural isomer threitol, comprising: a) mixing a biological sample from a subject with at least one stable isotope labeled structural isomer (SIL isomer) family member selected from: stable isotope labelled erythritol (SIL-erythritol) and SIL-threitol; b) mixing said biological sample with a hydroxyl group modifying agent such that said SIL isomer and erythritol and any threitol present in said biological sample are modified, thereby generating modified-erythritol, modified-threitol, and modified SIL isomer; c) subjecting the biological sample to purification such that the modified-erythritol, modified-threitol, and the modified SIL isomer are substantially isolated from other components of the biological sample, thereby generating a purified sample; and d) subjecting the purified sample to mass spectrometry such that peak intensity values are generated for the modified-erythritol, modified-threitol, and the modified SIL isomer; and e) employing the peak intensity value of the modified SIL isomer as an internal standard, and the peak intensity of the modified-erythritol, to determine the level of the erythritol present in the biological sample.

In certain embodiments, the subject has, or is suspected of having, cardiovascular disease, or has an increased risk of: developing cardiovascular disease, having a major adverse cardiovascular event, risk of stroke, or having enhanced thrombosis. In other embodiments, the biological sample is selected from a urine sample, plasma sample, blood sample, serum sample, sputum, or liquified stool sample. In other embodiments, the hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride. In particular embodiments, the modified-erythritol, modified SIL isomer, and modified-threitol are esterified or acetylated versions of said erythritol, SIL isomer, and threitol. In some embodiment, esterification reactions for polyol sweeteners are performed using the same chemistry as for cyclodextrin (e.g., Blaj et al., Molecules 2023, 28(5), 2001; Garcia, et al., PLoS ONE 2014, 9, e88234; and Li et al., Carbohydr. Res. 2015, 404, 55-62; all three of which are herein incorporated by refence.

In particular embodiments, the subject reports a higher than average intake of erythritol in their diet. In other embodiments, the at least one stable isotope labeled structural isomer comprises stable isotope labelled erythritol (SIL-erythritol). In some embodiments, the at least one stable isotope labeled structural isomer comprises stable isotope labelled threitol (SIL-threitol).

In further embodiments, the methods further comprise: generating a report comprising the level of erythritol, wherein the report identifies the level of erythritol as higher than a control level. In particular embodiments, the report describes that the subject should reduce intake of erythritol in order to improve: risk of cardiac disease and/risk of thrombosis. In other embodiments, the report describes that the subject should be prescribed and/or administered at least one of the following: a CVD therapeutic, an anti-coagulating agent, an anti-platelet agent, a lipid lowering agent, and a blood pressure control agent or therapy.

In particular embodiments, the mass spectrometry comprises MS/MS, and/or wherein the stable isotope is selected from C13 and deuterium (or other suitable isotope). In additional embodiments, the subject is a human.

In some embodiments, provided herein are methods comprising: a) receiving results of, or conducting, a circulating polyol sweetener analysis on a biological sample from a subject with, or suspected of having, or at increased risk of developing: a cardiovascular disease, a major adverse cardiovascular event, or enhanced thrombosis; and b) performing at least one of the following after identifying the biological sample as having higher levels of the polyol sweetener compared to control levels, i) treating the subject with a CVD therapeutic, anti-coagulating agent, anti-platelet agent; ii) treating the subject with lipid lowering agents, or BP control agent or therapy; iii) treating the subject with antiplatelet agents, due to the increased thrombosis risks associated with elevated polyol sweetener, iv) prescribing, or administering, to the subject a dietary intervention to reduce polyol sweetener levels in their diet; v) treating the subject with heart failure related therapy efforts; and/or vi) transmitting a report to the subject or medical personnel treating the subject, indicating the subject is suitable for, or should be, treated with any of i)-v) above.

In particular embodiments, provided herein are kits, systems, and composition comprising: a) at least one stable isotope labeled structural isomer (SIL isomer) family member selected from: stable isotope labelled xylitol (SIL-xylitol), SIL-arabitol, and SIL-rabitol; and b) at least one of the following: i) a mass spectrometer, ii) a hydroxyl group modifying agent; iii) a biological sample from a subject, optionally wherein the subject with, or suspected of having, or at increased risk of developing: a cardiovascular disease, a major adverse cardiovascular event, or enhanced thrombosis. In some embodiments, the mass spectrometer is present.

In certain embodiments, the hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.

In some embodiments, provided herein are kits, systems, or compositions comprising: a) at least one stable isotope labeled structural isomer (SIL isomer) family member selected from: stable isotope labelled erythritol (SIL-erythritol) and SIL-threitol; and b) at least one of the following: i) a mass spectrometer, ii) a hydroxyl group modifying agent, iii) a biological sample from a subject, optionally wherein the subject with, or suspected of having, or at increased risk of developing: a cardiovascular disease, a major adverse cardiovascular event, or enhanced thrombosis. In particular embodiments, the mass spectrometer is present. In other embodiments, the hydroxyl modifying group agent and/or biological sample is present. In other embodiments, the hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.

In certain embodiments, the polyol sweetener is erythritol or xylitol. In other embodiments, the polyol sweetener is selected from: isothreonic acid, threitol, pseudouridine, arabitol, myo-inositol, xylose, cellobiose, mannose, levoglucosan, saccharic acid, conduritol beta epoxide, lactulose, glycerol, 1,5-AHG, sucrose, beta-glycerol phosphate, glyceric acid, uridine, shikimic acid, 2-monoolein, threonic acid, fucose, UDP-glucuronic acid, fructose, ascorbate, maltose, inosine, glycerol-3-galactoside, propane-1,3-diol, glucose, 1,2-anhydro-myo-inositol, 1-monopalmitin, 6-deoxyyhexitol, 5-deoxyglucitol, glycerol-alpha-phosphate, 3-phosphoglycerate, 1-monstearin, 1-monolein, quinic acid, and diglycerol.

In some embodiments, the subject has at least one of the following: coronary artery disease (CAD), peripheral artery disease (PAD), cerebrovascular disease (CVD), transient ischemic attack (TIA), acute coronary syndrome (ACS), arterial aneurysm, heart failure (heart failure with preserved ejection fraction (HFpEF), heart failure with reduced ejection fraction (HFrEF)), need for revascularization (CABG, angioplasty, stent), or enhanced thrombosis, non-ST-segment myocardial infarction (NSTEMI), and ST-segment myocardial infarction (STEMI). In particular embodiments, the prescribing, or administering, to the subject a dietary intervention to reduce polyol sweetener levels in their diet comprises recommending to the subject at least one of the following: avoidance of artificial sweeteners, avoidance of processed foods, reduction in high glycemic index related foods, caloric restriction diet, and a weight loss diet. In further embodiments, the biological sample is selected from a urine sample, plasma sample, blood sample, serum sample, sputum, or liquified stool sample.

In additional embodiments, the anti-platelet agent is selected from: ASA, also called acetylsalicylic acid (Aspirin, Asaphen, Entrophen, Novasen); Clopidogrel (Plavix); Prasugrel (Effient); and Ticagrelor (Brilinta). In further embodiments, the anti-coagulant is selected from: a coumarin, a indandione, a factor Xa inhibitor, a heparin, a thrombin inhibitor, rivaroxaban (Xarelto), dabigatran (Pradaxa), apixaban (Eliquis), edoxaban (Lixiana), and Warfarin.

In further embodiments, the conducting a circulating polyol sweetener analysis comprises: i) mixing the biological sample from a subject with at least one stable isotope labeled structural isomer (SIL isomer) family member that includes the stable isotope labelled polyol sweetener and a first structural isomer of the polyol sweetener. In additional embodiments, the conducting a circulating polyol sweetener analysis further comprises: wherein said conducting a circulating polyol sweetener analysis further comprises: ii) mixing said biological sample with a hydroxyl group modifying agent such that said SIL isomer and said polyol sweetener are modified, thereby generating modified-polyol sweetener and modified SIL isomer. In additional embodiments, the conducting a circulating polyol sweetener analysis further comprises: iii) subjecting the biological sample to purification such that the modified-polyol sweetener and the modified SIL isomer are substantially isolated from other components of the biological sample, thereby generating a purified sample. In some embodiments, the conducting a circulating polyol sweetener analysis further comprises: iv) subjecting the purified sample to mass spectrometry such that peak intensity values are generated for the modified-polyol sweetener and the modified SIL isomer, and optionally further comprises: comprises: v) employing the peak intensity value of the modified SIL isomer as an internal standard, and the peak intensity of the modified-polyol sweetener, to determine the level of the polyol sweetener present in the biological sample.

In other embodiments, the conducting a circulating polyol sweetener analysis comprises: i) the use of mass spectrometry of the analytes; ii) the use of immunological detection of the analytes; iii) the use of colorimetric detection of the analytes (see, Musto et al., Analytical Chemistry. 2009; 81:6526-6533 and Sung et al., Org Lett. 2008 Oct. 16; 10(20):4405-8, both of which are herein incorporated by reference); iv) the use of electrochemical detection of the analytes (see, Filho et al., Food Chemistry. 2003; 83:297-301; and Nikolelis et al., Electrochimica Acta. 2001; 46:1025-1031, both of which are herein incorporated by reference); v) the use of fluorescence detection of the analytes (see, Zhang et al., Talanta. 2010; 81:591-596; and Li et al., A new fluorescent vesicular sensor for saccharides based on boronic acid-diol recognition on the interfaces of vesicles. 2007 SENSORS, 2007 IEEE, Print ISSN: 1930-0395; both of which are herein incorporated by reference herein), vi) the use of spectrophotometric detection of analytes (see, Capitin-Vallvey et al., Food Additives & Contaminants. 2004; 21:32-41 and Capitán-Vallvey et al., Valencia et al., Analytical and Bioanalytical Chemistry. 2006; 385:385-391); vii) the use of chemiluminescence detection of analytes (see, Niu et al., Analytical and Bioanalytical Chemistry. 2011; 402:389-395, herein incorporated by reference); viii) the use of ambient ionization mass spectrometry to detect analytes (see, Sisco et al., Analyst. 2015; 140:2785-2796; Nielen et al., TrAC Trends in Analytical Chemistry. 2011; 30:165-180; and Hajslova et al., TrAC Trends in Analytical Chemistry. 2011; 30:204-218; all of which are herein incorporated by reference). In certain embodiments, the analysis of the sample is LC-MS/MS or an antibody based assay (e.g., antibodies to erythritol are known, see Sreenath et al., Food Addit Contam. 2006 October; 23(10):1053, herein incorporated by reference, including for the antibody disclosed therein). In some embodiments, antibody based assays immunodiagnostic or competitive immunodiagnostic for erythritol or other polyol sweetener.

In some embodiments, the report comprises a paper report or an electronic report; and/or wherein the receiving information comprises receiving the report, wherein the receiving the report is optionally via: 1) the mail system, 2) email, or 3) via a LAN of a hospital or clinic. In other embodiments, the transmitting the report comprises: 1) mailing the reporting through the mail system, 2) emailing the report over the internet, or 3) sending the report through a local area network (LAN) or a hospital or clinic. In particular embodiments, the control value is derived from a sample from the general public or from a group known to not have cardiovascular disease or be at elevated risk for a thrombotic event. In further embodiments, the conducting a circulating polyol sweetener analysis is performed with an analytical device selected from: a mass spectrometer, NMR spectrometer, and a UV/Vis spectrometer.

### DESCRIPTION OF THE FIGURES

FIG. 1. Xylitol levels are associated with higher risks of major adverse cardiovascular events (MACE) in the Discovery and Validation Cohorts. Panel A (left) Circulating semi-quantitative xylitol levels (from untargeted metabolomics) in Discovery Cohort subjects. Boxes represent interquartile ranges (IQR) with the notch indicating the median. Lower whiskers represent smallest observation (≥25% quantile−1.5×IQR) and upper whiskers largest observation (≤75% quantile+1.5×IQR). Two-tailed Mann Whitney P values are indicated. (middle) Kaplan-Meier Plot for 3 year MACE stratified by tertiles (T) of relative levels of xylitol in Discovery Cohort subjects. P values were calculated with log rank test. (right) Hazard ratios (HR) for incident 3 year MACE based on univariable and multivariable Cox proportional-hazards regression analysis. Data points indicate HR, 95% confidence intervals are represented by line length. Multivariable adjustments include age, sex, smoking, diabetes, systolic blood pressure, LDL cholesterol, HDL cholesterol, triglycerides and hsCRP. Panel B (left) Circulating xylitol levels (from quantitative stable isotope dilution LC-MS/MS analysis) in Discovery Cohort subjects. Boxes represent interquartile ranges (IQR) with the notch indicating the median. Lower whiskers represent smallest observation (≥25% quantile−1.5×IQR) and upper whiskers largest observation (≤75% quantile+1.5×IQR). Two-tailed Mann Whitney P values are indicated. (middle) Kaplan-Meier Plot for 3 year MACE stratified by quartiles (Q) of plasma xylitol levels in the Validation Cohort. P values were calculated with log rank test. (right) Hazard ratios (HR) for incident 3 year MACE based on univariable and multivariable Cox proportional-hazards regression analysis. Data points indicate HR, 95% confidence intervals are represented by line length. Multivariable adjustments include age, sex, smoking, diabetes, systolic blood pressure, LDL cholesterol, HDL cholesterol, triglycerides and hsCRP.

FIG. 2. Xylitol levels following oral challenge and effect of xylitol platelet responsiveness. Panel A—Study participants (n=10) were given 30 g of xylitol dissolved in water to ingest. Xylitol levels were quantified via LC-MS/MS in the blood before and at the indicated times after the xylitol challenge in the first 4 subjects. In the remainder of subjects, xylitol levels were measured before and 30 min after xylitol challenge. Values listed above data points at each time point represent median(interquartile ranges). The distribution of fasting (≥12 h) plasma xylitol levels observed in the Validation Cohort are also shown: the dashed lines represent the upper and lower range, and the dotted lines indicate the bottom boundaries at quartiles (Q) 2, 3 and 4 in the Validation Cohort. Panel B and C Platelet rich plasma (PRP) was isolated from healthy volunteers and used to study the effects of varying levels of xylitol on agonist induced platelet aggregometry. Scatter plots show aggregometry responses for fixed concentrations of xylitol (30 μM, red circles) vs. vehicle (blue circles) with different concentrations of ADP (Panel A) or thrombin receptor-activating peptide (TRAP6, Panel B) with line representing medians. Global P values (for xylitol effect) were calculated with 2 way ANOVA and Šídák's multiple comparisons test to compare groups. *P<0.05, **P<0.01, ***P<0.001. Bar graphs (magnified areas) show submaximal ADP-stimulated (2 μM, Panel A) and TRAP6-stimulated (5 μM, Panel B) platelet aggregometry responses of human PRP following incubation with xylitol (30 μM, red) versus normal saline (vehicle, blue), with line and whiskers representing means (±SD). P values were calculated by two-tailed Mann Whitney Test. Panel D and E Aggregometry responses of human PRP with varying concentrations of xylitol and fixed submaximal concentration of ADP (2 μM, Panel D) and TRAP6 (5 M, Panel E) with lines and whiskers representing medians (IQR)P values were calculated by two-sided Kruskal Wallis test with Dunn's post hoc test. *P<0.05, **P<0.01, ***P<0.001. Bar graph data are represented as means (±SD). P values were calculated by two-tailed Mann Whitney Test.

FIG. 3. Xylitol increases stimulus-dependent intracellular calcium release and markers for activation in human platelets. Panel A—Representative fluorescent signal showing thrombin (0.02 U)-induced changes in intracellular calcium release in Fura 2-filled washed human platelets incubated with xylitol. Panel B—Fold-change (relative to vehicle) in peak Fura 2 fluorescence following submaximal (0.02 U) thrombin stimulation at the indicated concentrations of xylitol in washed human platelets. Bars show mean with SEM indicated by whiskers. P values were calculated by two-sided Kruskal Wallis test with Dunn's post hoc test. *P<0.05; **P<0.01; ***P<0.001. Panel C—ADP-induced changes in P-selectin surface expression in washed human platelets pre-incubated with the indicated concentrations of xylitol. Plotted are interquartile ranges (boxes). The line in the box is the median, whiskers represent minimum and maximum values. P values were calculated by two-sided Kruskal Wallis test with Dunn's post hoc test. *P<0.05; **P<0.01; ***P<0.001, ****P<0.0001. Panel D—ADP-induced changes in GP IIb/IIIa (PAC-1 antibody staining) in washed human platelets pre-incubated with the indicated concentrations of xylitol. Plotted are interquartile ranges (boxes). The line in the box is the median, whiskers represent minimum and maximum values. P values were calculated by two-sided Kruskal Wallis test with Dunn's post hoc test. *P<0.05; **P<0.01; ***P<0.001, ****P<0.0001. Panel E (left) Representative fluorescent images of platelet-leukocyte aggregates (BF, bright field, CD45 in green, P-selectin in yellow, CD41 in red, merged image) in human whole blood stimulated with TRAP6 (7.5 μM). (right) Numbers of platelet-leukocyte aggregates (CD45+, P-selectin+, CD41+) quantified by image stream in human whole blood incubated with indicated concentrations of xylitol at baseline (blue circles) and stimulated with 7.5 μM TRAP6 (red circles) relative to vehicle control with TRAP6. P values were calculated by two-sided Kruskal Wallis test with Dunn's post hoc test. *P<0.05; **P<0.01; ***P<0.001.

FIG. 4. Xylitol enhances in vivo clot formation. Panel A—Human platelet adhesion in whole blood to a collagen-coated microfluidic chip surface under physiological shear conditions ±xylitol. Representative images of platelet (green) adhesion at the indicated times (scale bar, 50 μm). P values were calculated by 2-way repeated measures ANOVA with Sidak's post hoc test. Overall P value (xylitol effect) is shown in black, Šídák's post hoc test P values are shown in red over the 3 follow-up times. Data is represented as means (±SEM). Panel B—Representative micrographs of carotid artery thrombus formation at the indicated time points following FeCl3-induced carotid artery injury (scale bar, 200 μm) and time to cessation of blood flow in mice from indicated groups i.p. injected with vehicle or xylitol. Bars represent means, two-sided P values were calculated by Mann Whitney Test. Plasma xylitol concentrations in both groups are indicated as means (±SEM).

FIG. 5. Effect of routine dietary xylitol challenge on platelet responsiveness in healthy subjects. Panel A and B Study participants (n=10) were given 30 g of xylitol in a drink. Before and 30 min after the xylitol challenge, PRP was rapidly isolated and platelet aggregometry performed using different concentrations of ADP (Panel A) and TRAP6 (Panel B). Shown are aggregation responses of paired samples (baseline and post xylitol) that were analyzed together. Boxes represent interquartile ranges (IQR) with the notch indicating the median. Lower whiskers represent smallest observation (≥25% quantile−1.5×IQR) and upper whiskers largest observation (≤75% quantile+1.5×IQR). The total number of replicates (multiple replicates per donor) and the total number of individual donors for each agonist concentration are indicated. P values were calculated with the Dunn test—a Kruskal-Wallis (K.W.) test with a Dunn post hoc analysis.

FIG. 6. Effect of dietary xylitol exposure on platelet responsiveness in individual subjects. Panel A and B—Platelet aggregation responses in PRP from each subject in response to submaximal concentration of ADP (2 M, Panel A) and TRAP6 (7.5 M, Panel B) before and after xylitol exposure. Shown are aggregation responses of paired samples (baseline and post xylitol) that were analyzed together. Boxes represent interquartile ranges (IQR) with the notch indicating the median. Lower whiskers represent smallest observation (≥25% quantile−1.5×IQR) and upper whiskers largest observation (≤75% quantile+1.5×IQR). The total number of replicates per individual donor is indicated. All subjects showed significant differences in agonist-induced aggregation (p<0.05) for pairwise comparison (pre vs. post xylitol exposure) except for subject 1 with only 3 replicates and subject 10 with 4 replicates showed p=0.07 for TRAP6 stimulation. P values were calculated with the Dunn test—a Kruskal-Wallis (K.W.) test with a Dunn post hoc analysis.

FIG. 7 shows a graphical overview of how artificial sweeteners can cause heart attack, stroke, or death by causing increased platelet reactivity (e.g., leading to thrombus formation).

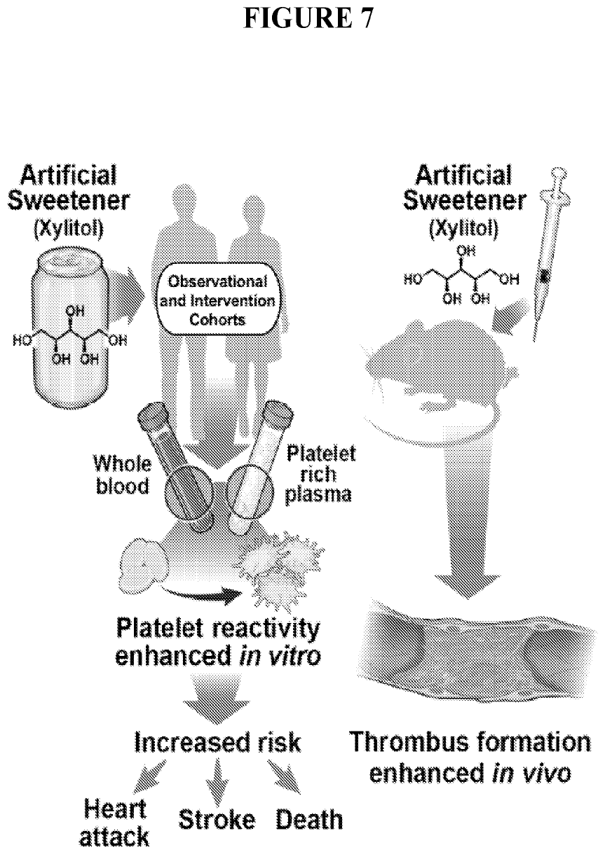

FIG. 8. Chromatographic separation of xylitol from its structural isomers arabitol and rabitol. Upper panel—Prior to chromatographic isolation and tandem mass spectrometry analyses, the structural isomers xylitol, arabitol and rabitol were first chemically derivatized by exhaustive acetylatation. They were then monitored by a characteristic parent 4 daughter ion transition shared amongst all three structural isomers. Shown for illustrative purposes is the structure of xylitol before and following acetylation, as well as the ammoniated ion form of the parent, and the origin of the daughter ion monitored after the loss of one OAc moiety and the ammonium ion. Middle and lower panels—Demonstration of baseline chromatographic separation of xylitol, arabitol and rabitol during LC-MS/MS analysis. Shown are individual chromatograms monitored following injection of each individual synthetic standard (middle) using multiple reaction monitoring (MRM) transition m/z 380→303 Da. The LC-MS/MS chromatogram of all three C5 polyol isomers (xylitol, arabitol and rabitol) when co-injected are baseline separated on a reverse phase column (after exhaustive acetylation). The MRM transition m/z 380→303 Da detects all three structural isomers. Also shown is the LC-MS/MS chromatogram monitored at MRM transition m/z 382→305 Da in the same sample spiked with synthetic [13C2]arabitol, which was used as internal standard. The accuracy and precision of the stable isotope dilution LC-MS/MS method developed to quantify xylitol (separated from its structural isomers arabitol and rabitol) was verified by the method of standard additions using authentic synthetic xylitol added to plasma samples, as described in Methods.

FIG. 9. Long-term risk of MACE among patient subgroups in the Validation Cohort. Shown is the HR for 3-year MACE based on the Cox proportional hazards regression analysis comparing top to bottom quartiles (Q). Data points (open circles) in the center indicate HR (with point estimates shown to the right) and 95% confidence intervals are represented by line length. MPC, mean platelet concentration; MPV, mean platelet volume; PCT, plateletcrit; PDW, platelet distribution width; PLT, platelet count. Antiplatelet drugs were defined as either aspirin or a P2Y12 agent.

FIG. 10. Postprandial levels of xylitol in urine. Xylitol levels were quantified via LC-MS/MS in the urine before, 30 min, 1 h, 4 h, 6 h and 24 h after the xylitol challenge in the first 4 subjects. In the remainder of subjects xylitol urine levels were measured before and 30 min after xylitol challenge. Urine xylitol levels shown are normalized to urine creatinine.

FIG. 11. Post prandial levels of xylitol and erythritol in mice reveal xylitol is poorly absorbed. Mice were gavaged with roughly molar equivalent levels of either xylitol alone (500 mg/kg in water) or erythritol alone (400 mg/kg), or both compounds (500 mg/kg xylitol and 400 mg/kg erythritol). Time-dependent changes in levels of xylitol and erythritol were quantified using LC-MS/MS in plasma (Panel A), urine (Panel B), and feces (Panel C) for the three groups of mice. At baseline, both xylitol and erythritol are present in mouse plasma at low levels (0.5-1.0 μM). After gavage (t=0), plasma levels of xylitol (left) did not exceed 4 μM, whereas erythritol levels show >100-fold increases. Significant xylitol increases also were not observed in the urine of mice post prandially (compared to erythritol). Fecal analyses reveal the majority of xylitol is recovered in feces, consistent with poor oral absorption. Reported is content of xylitol and erythritol per ug of wet feces.

FIG. 12. ADP-induced platelet aggregation before and after xylitol exposure. Study participants (n=10) were given 30 g of xylitol dissolved in water. Before and 30 min after the xylitol challenge, PRP was isolated and aggregation induced with different concentrations of ADP. Data for individual subjects are shown. Boxes represent 25th and 75th percentile with the notch indicating the median. Whiskers show minimum and maximum values. P values were calculated for the overall xylitol effect with Kruskal-Wallis (K.W.) rank sum test.

FIG. 13. TRAP6-induced platelet aggregation before and after xylitol exposure. Study participants (n=10) were given 30 g of xylitol dissolved in water. Before and 30 min after the xylitol challenge, PRP was isolated and aggregation induced with different concentrations of TRAP6. Data for individual subjects are shown. Boxes represent 25th and 75th percentile with the notch indicating the median. Whiskers show minimum and maximum values. P values were calculated for the overall xylitol effect with Kruskal-Wallis (K.W.) rank sum test.

FIG. 14. Correlation of xylitol levels with agonist-induced platelet aggregation. Panel A Scatter plots show submaximal agonist-induced aggregation responses for ADP (2 μM) and plasma xylitol concentrations of the same samples in the xylitol challenge study (FIG. 5) including all participants (n=10) before and after exposure to 30 g of xylitol. Spearman correlation rho values and P values are shown. Panel B Scatter plots show submaximum agonist-induced aggregation responses for TRAP6 (7.5 M) and plasma xylitol concentrations of the same samples including all participants (n=10) before and after exposure to 30 g of xylitol. Spearman correlation rho values and P values are shown.

FIG. 15. Kaplan-Meier estimates and forest plots indicating the risks of Major Adverse Cardiovascular Events (MACE), according to erythritol quartile level. Data shown are for the discovery cohort (upper panel), and two validation cohorts (US cohort, middle panel and European cohort, lower panel). The adjustment in discovery and US cohort included age, sex, type 2 diabetes, systolic blood pressure, body mass index (BMI), low-density and high-density lipoprotein cholesterol, triglyceride, and current smoking status. In the European Cohort, the adjustment included all of the aforementioned variables except for BMI (not available), and instead of systolic blood pressure, hypertension was used. Hazard ratios are indicated by data points in the centre (open circles). The 5-95% confidence interval is indicated by line length.

FIG. 16. Long term risk of Major Adverse Cardiovascular Events (MACE) among patient subgroups. Hazard ratios (HR) for 3 year MACE based on Cox proportional-hazards regression analysis compare top to bottom quartiles (Q) for the US cohort (left panel) and European cohort (right panel). Data points (open circles) in the centre indicate HR (with point estimates shown to the right), 95% confidence intervals are represented by line length. N numbers for each subgroup are indicated. P values for interaction with the groups and tabular data are shown in Table 14 and 15.

FIG. 17. Erythritol enhances platelet responsiveness. A. Bar graphs show submaximal ADP-stimulated (2 M) and Thrombin receptor-activating peptide 6 (TRAP6)-stimulated (5 μM) platelet aggregometry responses of human platelet-rich plasma following incubation with erythritol (45 M, red) versus normal saline (vehicle, blue). Data are represented as means (±SD), and P values were calculated by two-tailed Mann Whitney Test. Scatter plots show aggregometry with varying concentrations of erythritol and fixed submaximal level of ADP (2 μM) or TRAP6 (5 μM) including the data that is used in the bar graphs. P values were calculated by two-sided Kruskal Wallis test with Dunn's post hoc test. For ADP-stimulated PRP, n=15 for vehicle, n=6 for Erythritol 4.5 M, n=1 for Erythritol 18 M, n=10 for Erythritol 45 M, n=6 for Erythritol 90 and 270 PM. For TRAP6-stimulated PRP, n=10 for vehicle, n=6 for Erythritol 4.5 M, n=10 for Erythritol 18 μM, n=6 for Erythritol 45, 90 and 270 μM. (*p<0.05; **p<0.01; ***p<0.001). Dunn's adjusted P values for ADP-stimulated PRP (Erythritol vs. vehicle): Erythritol 45 M P=0.002, Erythritol 90 μM P=0.0008, Erythritol 270 M P<0.0001. Dunn's adjusted P values for TRAP6-stimulated PRP (Erythritol vs. vehicle): Erythritol 45 μM P=0.02, Erythritol 90 M P=0.002, Erythritol 270 μM P<0.0001. B. Thrombin-induced (0.02 U) changes in intracellular calcium concentration [Ca²⁺] in Fura 2-filled washed human platelets incubated with erythritol. P values were calculated by two-sided Wilcoxon matched-pairs signed rank test. n=11 per group. C. ADP-induced changes in GP IIb/IIIa (PAC-1 antibody staining) and P-selectin surface expression in washed human platelets pre-incubated with the indicated concentrations of erythritol. Boxes show 25th and 75th percentiles. The line in the box (centre) is the median, whiskers represent minimum and maximum values. P values were calculated by two-sided Kruskal Wallis test with Dunn's post hoc test for all samples. For GP IIb/IIIa activation, n=7 for ADP-stimulated PRP exposed to erythritol 4.5 μM, for all other conditions n=8 per group. For P-selectin surface expression n=8 per group. (*p<0.05; **p<0.01; ***p<0.001). Dunn's adjusted P values for GP IIb/IIIa activation (Erythritol vs. vehicle): Erythritol 4.5 μM P=0.04, Erythritol 18 M P=0.02, Erythritol 45 M P=0.003, Erythritol 90 M P=0.002, Erythritol 270 M P<0.0001. Dunn's adjusted P values for P-selectin surface expression (Erythritol vs. vehicle): Erythritol 18 μM P=0.03, Erythritol 45 μM P=0.04, Erythritol 90 μM P=0.005, Erythritol 270 M P=0.001. Each data point represents an individual measurement or the average of multiple measurements of a distinct sample. There were no repeated measurements within the data shown.

FIG. 18. Erythritol enhances in vivo thrombosis formation. A. Human platelet adhesion in whole blood to a collagen-coated microfluidic chip surface under physiological shear conditions ±erythritol. Individual biological samples we used and followed over 3 min. Representative images of platelet (green) adhesion at the indicated times (scale bar, 50 μm). P values were calculated by 2-way repeated measures analysis of variance with Sidák's post hoc test. Overall P value (erythritol effect) is shown in black, Sidák's post hoc test P values are shown in red over the 3 follow-up times. Data is represented as means (±SEM). n=10 for erythritol, n=11 for vehicle, n=3 for no collagen control. B. Representative micrographs of carotid artery thrombus formation at the indicated time points following FcCl₃-induced carotid artery injury (scale bar, 200 m) and time to cessation of blood flow in mice from indicated groups i.p. injected with vehicle or erythritol. Bars represent means, two-sided P values were calculated by Kruskal Wallis test with Dunn's post hoc test. n=11 for vehicle, n=12 for erythritol, n=8 for 1,5 anhydroglucitol (AHG).

FIG. 19. Effects of an Erythritol challenge on mean plasma levels. Study participants (n=8) were given 30 g of erythritol in a drink, and plasma levels were measured over the course of 7 days. Thresholds indicated (red) represent the erythritol concentrations noted in dose-response studies where significant increase in the indicated measure of platelet responsiveness was observed.

FIG. 20: Polyol metabolites and major adverse cardiovascular events (MACE) in untargeted metabolomics analyses of the discovery cohort. Shown are boxplots with relative levels for the indicated polyol (defined as compounds with two or more hydroxyl groups) area in both patients with (red) and without (blue) incident (3 yr) MACE ranked by Mann Whitney P values. Compound relative areas are shown as log of fold change (no MACE vs. MACE) to facilitate comparison. Boxes represent interquartile ranges (IQR) with the notch indicating the median. Lower whiskers represent smallest observation (≥25% quantile−1.5×IQR) and upper whiskers largest observation (≤75% quantile−1.5×IQR). Two-sided P values were calculated by Mann-Whitney U-test. N for no MACE=1041, n for MACE=116. False discovery rate corrected two-sided P values (Benjamini-Hochberg method) are indicated as follows: ****P<0.0001, ***P<0.001, **P<0.01, *P<0.05.

FIG. 21: Chromatographic separation of erythritol from its structural isomer threitol. After exhaustive acetylation with acetic acid anhydride, the polyols erythritol and its structural isomer, threitol, were baseline resolved by the HPLC method developed. Shown are the chromatograms generated by multiple reaction monitoring transitions (MRM) for the derivatized plasma analytes (m/z 308; [M+NH4]+) and synthetic isotopically labeled erythritol internal standard (D6-Erythritol; m/z 314; [M+NH4]+). With the column matrix and mobile phase/gradient employed, coupled with the characteristic parent [M+NH4+]→daughter ion transition used (for both erythritol and threitol), baseline chromatographic resolution of the two structural isomers was achieved.

FIG. 22. Plasma levels of erythritol are elevated in patients with major adverse cardiovascular events (MACE) and coronary artery disease (CAD) in both US and European validation cohorts. Erythritol levels in patients stratified by presence of (3 year) MACE or CAD. Data are shown as log of plasma Erythritol. Plotted are individual values as dots. Boxes represent interquartile ranges (IQR) with the notch indicating the median. Lower whiskers represent smallest observation (≥25% quantile−1.5×IQR) and upper whiskers largest observation (≤75% quantile−1.5×IQR). Two-sided P values were calculated by Mann-Whitney U-test. Numbers of subjects within each group are indicated.

FIG. 23. Erythritol increases platelet aggregation responses to submaximal concentrations of agonists. ADP-stimulated and Thrombin receptor-activating peptide(TRAP)6-stimulated platelet aggregometry responses of human platelet-rich plasma with fixed concentration of erythritol (45 or 90 M, red) versus normal saline (vehicle, blue). Data in bar graphs are represented as means (±SD), and two-sided P values were calculated by Mann Whitney Test (bar graphs) and by 2-way analysis of variance (overall P value is shown for erythritol effect) with Sidák's post hoc test. Sidák's adjusted P values for Erythritol 45 μM vs. vehicle: for ADP 2 μM P=0.01, ADP 3 μM P=0.005, for erythritol 90 μM vs. vehicle: TRAP6 5 μM: P=0.0002. Numbers of independent biological replicates (n) are indicated. *P<0.05, **P<0.01, ***P<0.001.

FIG. 24. Impact of glucose on platelet aggregation. ADP-stimulated (left panel) and Thrombin receptor-activating peptide (TRAP) 6-stimulated (right panel) platelet aggregometry responses in human platelet-rich plasma incubated with glucose (270 M, green) versus vehicle (saline, blue). Data in bar graphs are represented as means (±SD). Two-sided P values were calculated using Mann-Whitney U-test. Numbers of independent biological replicates (n) are indicated.

FIG. 25. Impact of 1,5 Anhydroglucitol (AHG) on platelet aggregation and calcium release. Panel A ADP-stimulated and Thrombin receptor-activating peptide (TRAP)6-stimulated platelet aggregometry responses in human platelet-rich plasma incubated with 1,5-AHG (green) versus vehicle (saline, blue). Two-sided P values were calculated by Mann Whitney Test. For ADP and TRAP6 stimulated platelet-rich plasma n=7. Panel B shows thrombin-induced (0.02 U) changes in intracellular calcium concentration in Fura 2-filled washed human platelets incubated with 1,5-AHG (green) or vehicle (saline, blue). Data represent mean (±SD). Two-sided P values were calculated by Wilcoxon matched-pairs signed rank test. Numbers of independent biological replicates (n) are indicated.

FIG. 26. Impact of 1,5-Anhydroglucitol (AHG) and glucose on platelet activation. ADP-induced changes in GP IIb/IIIa (PAC-1 antibody staining) and P-selectin surface expression in washed human platelets pre-incubated with vehicle (saline, blue) or the indicated concentrations of either 1,5-AHG (green, panel A) or glucose (green, panel B). Bars represent means (±SD), Two-sided P values were calculated by Kruskal-Wallis test with Dunn's post hoc test for multiple-group comparisons. Numbers of independent biological replicates (n) are indicated.

FIG. 27. Impact of erythritol at different physiological concentrations on platelet aggregation responses. Human platelet-rich plasma was incubated with erythritol (red) at low levels observed in fasting patients (18 μM) and higher concentrations observed after erythritol ingestions (6 mM) versus vehicle (saline, blue). Shown are thrombin receptor-activating peptide(TRAP)6-stimulated (panel A) and ADP-stimulated (panel B) platelet aggregometry responses. Data in bar graphs are represented as means (±SD). Two-sided P values were calculated by Mann Whitney Test. Numbers of independent biological replicates (n) are indicated.

FIG. 28: Example of the gating strategy used for platelet flow cytometry experiments. Human washed platelets were exposed to vehicle (saline), stimulated with 2 M ADP and incubated with anti-P-selectin antibody (CD62P-PE). The sample was analyzed on a FACS LSR Fortessa flow cytometer. Platelets were gated to exclude doublets and the raw mean fluorescent intensity (MFI) was quantified. Twenty thousand events were collected.

FIG. 29. Erythritol ingestion enhances platelet responsiveness to multiple agonists in healthy volunteers. Platelet aggregation in response to the indicated concentrations of ADP (top) or TRAP6 (bottom) at baseline (blue) and 30 min post glucose (orange) or erythritol ingestion (red). Multiple replicates of paired samples (connected by lines) from each subject are shown. Boxes represent interquartile range (IQR) with median (thicker line within box). Lower whiskers represent smallest observations (≥25% quantile−1.5×IQR), and upper whiskers represent largest observations (≤75% quantile+1.5×IQR). The total number of paired (baseline/postprandial) replicates from subjects for challenges (glucose or erythritol) are shown. P values for pairwise comparisons were performed with Wilcoxon signed rank test.

### DEFINITIONS

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject.

### GENERAL DESCRIPTION

Provided herein are compositions, systems, kits, and methods for determining the level of a polyol sweetener in a biological sample from a subject (e.g., a subject with, or suspected of having, or at increased risk of developing: a cardiovascular disease, a major adverse cardiovascular event, or enhanced thrombosis). In certain embodiments, determining such polyol sweetener levels are higher than a control level allows a subject to be prescribed, or administered, a CVD therapeutic, anti-thrombotic agent, or lifestyle change to reduce consumption of such polyol sweeteners. In certain embodiments, xylitol is detected in a biological sample from a subject such that xylitol's structural isomers arabitol and rabitol are distinguished. In other embodiments, erythritol is detected in a biological sample from a subject such that erythritol's structural isomer threitol is distinguished.

Work conducted during development of embodiments herein determined that polyol sweeteners, such as erythritol and xylitol, enhances platelet reactivity and thrombosis potential in subjects (e.g., healthy individuals and those with CVD risk). The use of “zero calorie,” “zero carb,” and “keto” friendly processed food products, which are often marketed towards subjects with heightened cardiovascular disease (CVD) risk, is exponentially increasing. Although artificial sweeteners used in these products have received “generally regarded as safe” (GRAS) status by US and EU regulatory agencies, and are recommended by professional health bodies (e.g. ADA, AHA, EU National health authorities), there have been no clinical trials to assess long-term CVD risks. Similarly, there have been no interventional studies that directly examine short-term relevant phenotypes like pro-thrombotic effects of artificial sweeteners.

As shown in the Examples below, erythritol consumption enhanced platelet reactivity and thrombosis potential in every subject examined. Such present results provide evidence for a causal relationship between erythritol ingestion and heightened thrombosis potential. The present results provide have far-reaching clinical and public health implications. Erythritol has become one of the most widely used sugar substitutes worldwide, and our results indicate that consumption of a typical sized portion of an erythritol sweetened drink will foster a pro-thrombotic effect. While use of sugar substitutes is intended to protect individuals from excess calories and dietary glucose, the very subjects most likely to use sugar substitutes are often the most vulnerable to experiencing heightened CVD risks. Our work provides evidence for a causal relationship between artificial sweetener consumption in general, and erythritol consumption specifically, and thrombosis potential, and thus provide a mechanistic explanation for previously noted clinical associations between circulating levels of erythritol and incident risks for major adverse CVD events.

In certain embodiments, levels of the polyol sweetener (e.g., xylitol or erythritol) in the biological sample obtained from the subject may compared to a control value (e.g., to know if the polyol sweetener level is the same, increased, or decreased compared to the control). A control value is a concentration of an analyte that represents a known or representative amount of an analyte. For example, the control value can be based upon levels of the selected polyol sweetener in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of cardiovascular disease or atherosclerosis, such as angina pectoris, or history of an acute adverse cardiovascular event such as a myocardial infarction or stroke, or evidence of atherosclerosis by diagnostic imaging methods including, but not limited to coronary angiography. In certain embodiments, the reference cohort includes individuals, who if examined by a medical professional would be characterized as free of symptoms of disease (e.g., cardiovascular disease). In another example, the reference cohort may be individuals who are nonsmokers (i.e., individuals who do not smoke cigarettes or related items such as cigars). Accordingly, the control values selected may take into account the category into which the test subject falls. Appropriate categories can be selected with no more than routine experimentation by those of ordinary skill in the art.

The control value is preferably measured using the same units used to characterize the level of the polyol sweetener obtained from the subject. Thus, if the level of the selected polyol sweetener is an absolute value such as the units of the polyol sweetener per ml of blood or plasma, the control value is also based upon the units of the polyol sweetener per ml of blood or plasma in individuals in the general population or a select population of human subjects.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Control values of the selected polyol sweetener in biological samples obtained, such as mean levels, median levels, or “cut-off levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described, for example, in Knapp, R. G., and Miller, M. C. (1992). Clinical Epidemiology and Biostatistics. William and Wilkins, Harual Publishing Co. Malvern, Pa., which is specifically incorporated herein by reference. A “cutoff value can be determined for each GDAAM that is assayed.

In some embodiments, a subject with an elevated level of an artificial sweetener in a sample is treated with a cardiovascular disease or anti-thrombotic agent. In certain embodiments, the cardiovascular disease therapeutic is a lipid lowering agent, and optionally wherein the lipid lowering agent comprises a statin. In additional embodiments, the cardiovascular disease therapeutic is selected from: i) atorvastatin, rosuvastatin, simvastatin, pravastatin, lovastatin, pitavastatin, bempadoic acid, ii) a PCSK9 inhibitor, wherein the PCSK9 inhibitor optionally comprises a anti-PCSK9 antisense or siRNA, or an anti-PCSK9 antibody, fragment thereof, or nanobody, iii) an apoB inhibitor, wherein the apoB inhibitor optionally comprises a anti-apoB antisense or siRNA, or an anti-apoB antibody, fragment thereof, or nanobody; or iv) lipoprotein a or apolipoprotein a.

In certain embodiments, the sample is selected from the group consisting of: a plasma sample, a serum sample, a liquified stool sample, whole blood sample, and a urine sample. In other embodiments, the determining comprises detecting the level of a polyol sweetener is with an analytical device selected from: a mass spectrometer, NMR spectrometer, and a UV/Vis spectrometer.

In some embodiments, if the level of a polyol sweetener in the test subject's biological sample is greater than the control value or exceeds or is in the upper range of control values, the test subject is at greater risk of developing or having CVD or a thrombosis, or experiencing an adverse cardiac event within the ensuing year, two years, and/or three years than individuals with levels comparable to or below the control value or in the lower range of control values. In contrast, if levels of the polyol sweetener in the test subject's biological sample is below the control value or is in the lower range of control values, the test subject is at a lower risk of developing or having CVD or a thrombotic event or experiencing an adverse cardiac event within the ensuing year, two years, and/or three years than individuals whose levels are comparable to or above the control value or exceeding or in the upper range of control values. The extent of the difference between the test subject's polyol sweetener level and control value is also useful for characterizing the extent of the risk and thereby determining which individuals would most greatly benefit from certain aggressive therapies or greatly reducing polyol sweetener intake in their diet. In those cases, where the control value ranges are divided into a plurality of groups, such as the control value ranges for individuals at high risk, average risk, and low risk, the comparison involves determining into which group the test subject's level of the relevant risk predictor falls.

Another type of control value is an internal standard in the sample. An internal standard is a known amount of another compound that can be provided in a sample that can be measured along with the analyte to serve as a reference. The diagnostic methods described herein can also be carried out by determining the levels of a selected polyol sweetener in a subject's biological sample and comparing them to the amount of an internal standard.

### EXAMPLES

### Example 1

Xylitol is Associated with Cardiovascular Event Risks and Enhances Platelet Responsiveness and Thrombosis Potential In Vivo

Low-calorie sweeteners are widely used sugar substitutes in processed foods with presumed health benefits. Here, we describe both clinical evidence linking endogenous levels of the sugar alcohol xylitol with incident major adverse cardiac event risk, and both animal model and human intervention studies with this common low-calorie sweetener that reveal a causal role for xylitol in fostering heightened platelet reactivity and thrombosis potential.

Initial untargeted metabolomics studies were performed in a Discovery Cohort of 1,157 sequential stable subjects undergoing elective diagnostic cardiac evaluations, and subsequent stable-isotope-dilution LC-MS/MS analyses were performed on an independent, non-overlapping Validation Cohort (n=2,149). Complementary in vitro isolated human platelet, platelet rich plasma, whole blood, and animal model studies examined the effect of xylitol on platelet responsiveness and thrombus formation in vivo. Finally, an intervention study (n=10) was performed to assess the effects of xylitol consumption on platelet function in human subjects.

In initial untargeted metabolomics studies (Discovery Cohort), circulating levels of a polyol tentatively assigned as xylitol were associated with incident (3 yr) major adverse cardiovascular events (MACE) risk. Subsequent stable-isotope-dilution LC-MS/MS analyses specific for xylitol (and not its structural isomers) confirmed its association with incident MACE risk (4th vs. 1st quartile adjusted hazard ratio[95% CI], 2.00[1.31-3.06]). Complementary mechanistic studies showed xylitol enhanced multiple indices of platelet reactivity and in vivo thrombosis formation. In interventional studies, consumption of a xylitol-sweetened drink was shown to enhance multiple measures of platelet responsiveness in subjects. Fasting plasma xylitol levels are associated with incident MACE risk. Elevated xylitol levels are shown to enhance platelet reactivity and promote thrombosis potential in vivo.

Xylitol is a 5-carbon sugar alcohol (polyol) that is commonly used as a low-calorie sweetener. It can be found in small quantities in fruits and vegetables²⁷ but is highly enriched (often over 1000-fold higher than found in nature) in numerous artificially sweetened foods and beverages. Because of its anti-cariogenic properties²⁸, xylitol is also commonly used in candy, gum and oral care products.²⁹ Xylitol is also produced endogenously as a low abundance intermediate side product of human glucose metabolism, and has negligible impact on blood sugar or insulin secretion.^{30,31} In contrast to traditional high intensity sweeteners, xylitol has comparable sweetness to sucrose.²⁹ Thus, when used as a sugar substitute, it is added in much larger amounts to processed food (i.e. levels equal to sucrose with up to 45 g per serving in some products such as artificially sweetened pie fillings).³² It is therefore appreciated by the food industry as a bulking sugar substitute that confers texture, moisture and increases shelf life of processed foods without after taste.³³ Consequently, xylitol is marketed as a “natural sweetener”, “keto friendly” or “low carb”, and is generally recommended as a sugar substitute for patients with diabetes to improve glycemic control.³⁴ Despite the ever growing market share and use of xylitol in processed foods and oral care products, the impact of xylitol on cardiovascular event risk has not been reported. Here we provide human clinical observational, interventional, and mechanistic studies linking the low-calorie sweetener xylitol to CVD event risk and both heightened platelet reactivity and a pro-thrombotic state in vivo.

### Methods

### Human Subjects

### Study Approvals

We performed 3 distinct clinical studies. All human subjects provided written informed consent, and all human studies abided by the Declaration of Helsinki. The Institutional Review Board of the Cleveland Clinic approved all human study protocols (GeneBank IRB 4265; IRB 21-005 (xylitol ingestion related studies), healthy volunteer blood donors for platelet-related studies IRB 09-506).

### Discovery Cohort Study Design

Our first study employed a Discovery Cohort, where we used untargeted metabolomics (semi-quantitative analyses) to analyze plasma samples from 1,157 stable subjects to identify circulating analytes whose levels were associated with future development of adverse cardiovascular events over an ensuing 3 year period. The Discovery Cohort included sequential stable patients (ages 18 years or above) undergoing elective diagnostic coronary angiography for risk evaluation at a quaternary referral center between 2001 and 2007 (GeneBank at the Cleveland Clinic; clinicaltrials.gov identifier: NCT00590200) with clinical and longitudinal outcomes.

### Validation Cohort Study Design

Our second clinical study employed an independent non-overlapping Validation Cohort, where we employed quantitative stable isotope dilution liquid chromatography tandem mass spectrometry (LC-MS/MS) to determine xylitol levels in serum samples from 2,149 patients (non-overlapping from GeneBank at the Cleveland Clinic; clinicaltrials.gov identifier: NCT0059020).³⁵⁻³⁶

In both, the Discovery and Validation Cohorts, Major Adverse Cardiovascular Events (MACE) were defined as death, nonfatal myocardial infarction, or nonfatal cerebrovascular accident (stroke) during follow-up. Adjudicated long term survival data were collected through medical records review and Social Security Death Index query. Cardiovascular disease (CVD) was indicated as presence of peripheral artery disease or coronary artery disease, indicated as any clinical history of myocardial infarction, coronary revascularization (including percutaneous coronary intervention, coronary artery bypass surgery), or angiographic evidence of significant stenosis (≥50%) in 1 or more major coronary arteries. Estimated glomerular filtration rate (eGFR) in both cohorts was calculated based on the recent Chronic Kidney Disease Epidemiology Collaboration 2021 CKD-EPI Creatinine equation.³⁷ Platelet-related hematology analyses were performed with an Advia 120 hematology analyzer (Siemens, New York, NY).³⁸ Use of anti-platelet agents (=1556) was defined as either aspirin or a P2Y12 agent or both. All observational studies in this example are reported in compliance with the STrengthening the Reporting of Observational studies in Epidemiology (STROBE)-Statement.³⁹ Information on gender of study participants of the Discovery and Validation Cohorts is reported in Table 1-3. Moreover, gender was included in our multivariable Cox models and did not affect the association of xylitol with incident risk for MACE.

TABLE 1
Clinic characteristics of the Discovery and Validation Cohorts.

| | Discovery Cohort | Validation Cohort |
|---|---|---|
| Characteristic | (n = 1,157) | (n = 2,149) |
| Age (years) | 65.0 (56-72) | 63.0 (55-72) |
| Male (%) | 63.6 | 64.0 |
| BMI (kg/m²) | 28.4 (25.4-32.1) | 28.4 (25.5-32.2) |
| Diabetes mellitus (%) | 22.0 | 22.1 |
| Hypertension (%) | 72.2 | 70.4 |
| Current smoking (%) | 13.7 | 12.7 |
| MACE 3 Years (%) | 10.0 | 10.2 |
| Cardiovascular disease (%) | 75.9 | 77.7 |
| Heart failure (%) | 16.8 | 19.4 |
| eGFR (mL/min/1.73 m²) | 89.8 (75.7-99.1) | 90.4 (75.4-100.3) |
| LDL cholesterol (mg/dL) | 96.0 (80.0-116.0) | 96.0 (77.0-117.0) |
| HDL cholesterol (mg/dL) | 34.3 (28.5-41.2) | 34.3 (28.2-41.7) |
| Total cholesterol (mg/dL) | 163.2 (142.7-188.2) | 160.8 (138.5-187.4) |
| Triglycerides (mg/dL) | 122.0 (84.0-171.0) | 114.0 (84.0-163.0) |
| Statins (%) | 61.4 | 59.1 |
| Aspirin (%) | 76.8 | 72.4 |
| Anti-diabetic drugs (%) | 9.2 | 12.1 |
| ACE inhibitors (%) | 49.9 | 49.9 |
| Calcium channel blockers (%) | 18.8 | 18.9 |
| Diuretics (%) | 18.6 | 20.5 |

The baseline characteristics of participants in the Discovery Cohort and Validation Cohort are shown. Continuous data are presented as median (interquartile range) and categorical variables are presented as %. ACE, angiotensin converting enzyme; BMI, body mass index; eGFR, estimated glomerular filtration rate; HDL, high density lipoprotein; LDL, low density lipoprotein; MACE, major adverse cardiovascular events.

TABLE 2
Clinic characteristics of the Discovery Cohort stratified by xylitol levels.

| | All subjects | Tertile 1 | Tertile 2 | Tertile 3 | |
|---|---|---|---|---|---|
| Characteristics | (n = 1157) | (n = 382) | (n = 393) | (N = 382) | P |
| Age (years) | 65.0 (56-72) | 65.5 (56-73) | 65.2 (56-72) | 64.3 (55-72) | 0.49 |
| Male (%) | 63.6 | 62.6 | 62.8 | 65.4 | 0.66 |
| BMI (kg/m²) | 28.4 (25.4-32.1) | 28.4 (25.3-31.7) | 28.6 (25.7-32.7) | 28.2 (25.3-31.7) | 0.25 |
| Diabetes mellitus (%) | 22.0 | 21.5 | 22.4 | 22.3 | 0.95 |
| Hypertension (%) | 72.2 | 68.3 | 72.8 | 75.5 | 0.09 |
| Current smoking (%) | 13.7 | 10.7 | 16.3 | 13.9 | 0.08 |
| MACE 3 Years (%) | 10 | 8.4 | 8.4 | 13.4 | 0.03 |
| Cardiovascular disease (%) | 75.9 | 74.9 | 74.6 | 78.3 | 0.41 |
| Heart failure (%) | 16.8 | 13.1 | 14.6 | 22.7 | 0.001 |
| eGFR (mL/min/1.73 m²) | 89.8 (75.7-99.1) | 89.5 (76.4-99.9) | 89.9 (76.9-98.8) | 90.3 (70.9-98.9) | 0.57 |
| LDL cholesterol (mg/dL) | 96.0 (80.0-116.0) | 98.0 (83.0-117.0) | 98.0 (81.0-119.0) | 92.0 (76.0-111.0) | 0.001 |
| HDL cholesterol (mg/dL) | 34.3 (28.5-41.2) | 35.0 (29.8-42.1) | 34.1 (28.1-40.9) | 33.5 (27.8-40.2) | 0.009 |
| Total cholesterol (mg/dL) | 163.2 (142.7-188.2) | 165.7 (145.7-188.7) | 166.7 (146.2-192.3) | 157.2 (133.9-182.5) | <0.001 |
| Triglycerides (mg/dL) | 122.0 (84.0-171.0) | 115.5 (81.3-160.8) | 132.0 (87.0-189.0) | 116.0 (84.0-167.8) | 0.005 |
| Statins (%) | 61.4 | 63.9 | 57.8 | 62.6 | 0.18 |
| Aspirin (%) | 76.8 | 79.3 | 74.6 | 76.7 | 0.29 |
| Anti-diabetic drugs (%) | 9.2 | 8.9 | 9.9 | 8.6 | 0.81 |
| ACE inhibitors (%) | 49.9 | 46.6 | 49.4 | 53.7 | 0.14 |
| Calcium channel blockers (%) | 18.8 | 16.8 | 20.6 | 18.8 | 0.39 |
| Diuretics (%) | 18.6 | 16 | 16.3 | 23.6 | 0.009 |

Continuous data are presented as median (interquartile range) and categorical variables are presented as %. ACE, angiotensin converting enzyme; BMI, body mass index; eGFR, estimated glomerular filtration rate; HDL, high density lipoprotein; LDL, low density lipoprotein; MACE, major adverse cardiovascular events. Difference between tertiles were examined using Kruskal Wallis test for continuous variables and chisq (χ²) test for categorical variables.

TABLE 3
Clinic characteristics of the Validation Cohort stratified by xylitol levels.

| | All subjects | Quartile 1 | Quartile 2 | Quartile 3 | Quartile 4 | |
|---|---|---|---|---|---|---|
| Characteristics | (n = 1157) | (n = 515) | (n = 557) | (n = 528) | (n = 549) | P |
| Age (years) | 63.0 | 60.9 | 63.9 | 63.5 | 63.8 | 0.005 |
| | (55.2-71.5) | (54.4-69.4) | (55.4-71.6) | (55.5-72.5) | (55.8-72.1) | |
| Male (%) | 64 | 64.1 | 64.3 | 68 | 59.9 | 0.05 |
| BMI (kg/m²) | 28.4 | 28.1 | 28.4 | 28.7 | 28.7 | 0.63 |
| | (25.5-32.2) | (25.4-31.7) | (25.7-32.0) | (25.4-32.2) | (25.4-32.9) | |
| Diabetes mellitus (%) | 22.1 | 19.6 | 22.6 | 20.3 | 25.7 | 0.07 |
| Hypertension (%) | 70.4 | 66.3 | 69.8 | 70.0 | 75.4 | 0.01 |
| Current smoking (%) | 12.7 | 13.6 | 11.0 | 10.8 | 15.5 | 0.06 |
| MACE 3 Years (%) | 10.2 | 6 | 9.9 | 11.2 | 13.7 | 0.001 |
| Cardiovascular disease | 77.7 | 73.4 | 80.4 | 79.2 | 77.4 | 0.04 |
| (%) | | | | | | |
| Heart failure (%) | 19.4 | 15.3 | 19.1 | 18.8 | 24 | 0.005 |
| eGFR (mL/min/1.73 m²) | 90.4 | 93.12 | 90.9 | 89.9 | 88.1 | <0.001 |
| | (75.4-100.3) | (81.6-101.4) | (77.8-100.3) | (72.3-99.6) | (71.8-99.0) | |
| LDL cholesterol (mg/dL) | 96.0 | 98.0 | 96.0 | 96.0 | 94.0 | 0.06 |
| | (77.0-117.0) | (78.0-124.0) | (79.0-115.0) | (77.0-117.3) | (75.0-113.0) | |
| HDL cholesterol (mg/dL) | 34.3 | 35.3 | 34.6 | 33.7 | 33.5 | 0.01 |
| | (28.2-41.7) | (29.1-42.9) | (28.5-43.0) | (28.0-40.6) | (27.5-40.5) | |
| Total cholesterol (mg/dL) | 160.8 | 160.7 | 163.4 | 159.4 | 158.7 | 0.07 |
| | (138.5-187.4) | (141.0-193.0) | (140.4-187.2) | (137.8-185.5) | (135.8-184.8) | |
| Triglycerides (mg/dL) | 114.0 | 111.0 | 111.0 | 117.5 | 117.00 | 0.55 |
| | (84.0-163.0) | (82.0-160.0) | (85.0-162.0) | (85.8-167.1) | (85.0-165.0) | |
| Statins (%) | 59.1 | 61.7 | 60 | 54.2 | 60.3 | 0.06 |
| Aspirin (%) | 72.4 | 74.6 | 74.5 | 70.8 | 69.6 | 0.15 |
| Anti-diabetic drugs (%) | 12.1 | 9.7 | 11.5 | 12.3 | 14.8 | 0.09 |
| ACE inhibitors (%) | 49.9 | 46.8 | 49 | 51.9 | 51.9 | 0.27 |
| Calcium channel blockers | 18.9 | 14.4 | 16.9 | 18.9 | 25.1 | <0.001 |
| (%) | | | | | | |
| Diuretics (%) | 20.5 | 14.4 | 18.7 | 21.6 | 27 | <0.001 |

Continuous data are presented as median (interquartile range) and categorical variables are presented as %. ACE, angiotensin converting enzyme; BMI, body mass index; eGFR, estimated glomerular filtration rate; HDL, high density lipoprotein; LDL, low density lipoprotein; MACE, major adverse cardiovascular events. Difference between quartiles were examined using Kruskal Wallis test for continuous variables and chisq (χ²) test for categorical variables.

### Xylitol Intervention Study Design

A third xylitol intervention study (clinicaltrials.gov number: NCT04731363) included 10 prospectively recruited healthy volunteers (35±11 years of age, 50% male, nonsmokers without CVD, hypertension, or diabetes). Fasting (overnight) subjects were provided a standardized artificially sweetened beverage (300 mL) containing 30 g of xylitol dissolved in water with instructions to rapidly consume the drink (within 2 minutes). In the first part of xylitol ingestion studies, pharmacokinetics studies focused on post-prandial plasma levels and time course of elimination of xylitol. For LC-MS/MS analysis of xylitol, serial venous blood and urine sampling was performed at baseline, 30 min, 1 h, 4 h, 6 h and 24 h to establish postprandial peak levels and the time course of xylitol elimination in the first 4 subjects. After we noticed that xylitol levels were rapidly cleared with postprandial xylitol peak levels at 30 min after ingestion in all 4 subjects, we streamlined our protocol in the interest of being both practical and less demanding on subjects, and sampled blood and urine at baseline and after 30 min for the remainder of subjects.

The second part of xylitol ingestion studies focused on platelet phenotypic changes after oral xylitol challenge. In these parallel studies, platelet-rich plasma (PRP) was isolated immediately after the blood draw (from the same subjects that were used for xylitol pharmacokinetics studies) and aggregometry assays performed (as described below) on blood samples collected at baseline and 30 min (when postprandial peak levels were observed) after xylitol exposure.

A non-randomized study design was used wherein each subject served as their own control, comparing both xylitol levels and indices of platelet function within subjects at baseline versus 30 min following ingestion of the standardized drink of xylitol. Power calculations were performed for our intervention study a priori and suggested a sample size of 10 per group with an expected standard deviation of approximately 20% for both postprandial xylitol levels (based on previous literature of polyol pharmacokinetics studies) and changes in aggregation responses (based on previous observations with aggregometry studies) to detect a change in 30% or more in the means, to a power of 0.9 with a type 1 error rate of 5%. Xylitol ingestion related clinical studies registration and IRB protocol also include an intervention study using an alternative sweetener (erythritol) that was performed independently with a distinct non-overlapping subset of subjects and that is not part of the current manuscript.

### Human Blood Donors for Platelet-Related Studies

Healthy adults (both male and female) with no chronic illness were consented for donation of blood for use in research (for other platelet and thrombosis related in vitro studies involving human platelets, PRP or whole blood). These samples were acquired using a distinct IRB protocol with no overlap to samples from our xylitol intervention study. All studies were approved by their local institutional board review and all participants provided written informed consent.

### Animals

Mice were used to causally test whether elevation in xylitol plasma levels results in enhancement in thrombosis potential. All mice were C57BL/6J and 12-14 weeks of age. All animal studies were approved by the Institutional Animal Care and Use Committee at the Cleveland Clinic (IRB 2019-2251).

### Aggregometry Studies in Platelet-Rich Plasma

Aggregometry in platelet rich plasma (PRP) was performed as previously described⁴⁰ and outlined in supplemental methods.

### Intracellular Calcium Measurements

Measurement of intracellular calcium release in washed platelets was performed as previously described.⁴¹ Further details are provided in supplemental methods.

### Platelet Flow Cytometry Assay

Antibody staining of washed platelets for flow cytometry was performed as described previously⁴¹ with further information outlined in additional methods.

### Imaging Flow Cytometry in Whole Blood

Imaging flow cytometry was performed on an Amnis ImageStreamX MK II two-camera system using anti-CD45 AF488, anti-CD41 AF647 and anti-CD62P-PE. Details and the gating strategy are described in supplemental methods.

### Whole Blood in Vitro Thrombosis Assay

Shear flow experiments of whole blood were performed with a Cellix Microfluidics System (Cellix, Dublin, Ireland) as previously described and outlined in additional methods.⁴¹⁻⁴³ The extent of platelet activation and adhesion to the collagen matrix was quantified using computer assisted tomographic analyses, as previously described.⁴⁰

### Carotid Artery FeCl₃ Injury Model

The FeCl₃-induced carotid artery injury model was performed using intravital fluorescence microscopy with continuous image capture monitoring, as previously described.⁴⁴ Time to cessation of blood flow through thrombus formation for all experiments was determined by visual inspection by two independent investigators.

### Untargeted and Targeted Mass Spectrometry Analyses of Human Plasma

For untargeted mass spectrometry analyses, subject plasma samples were derivatized and run on GC-MS analyses as previously described.¹⁹ Raw data files were processed using the metabolomics BinBase database.⁴⁵

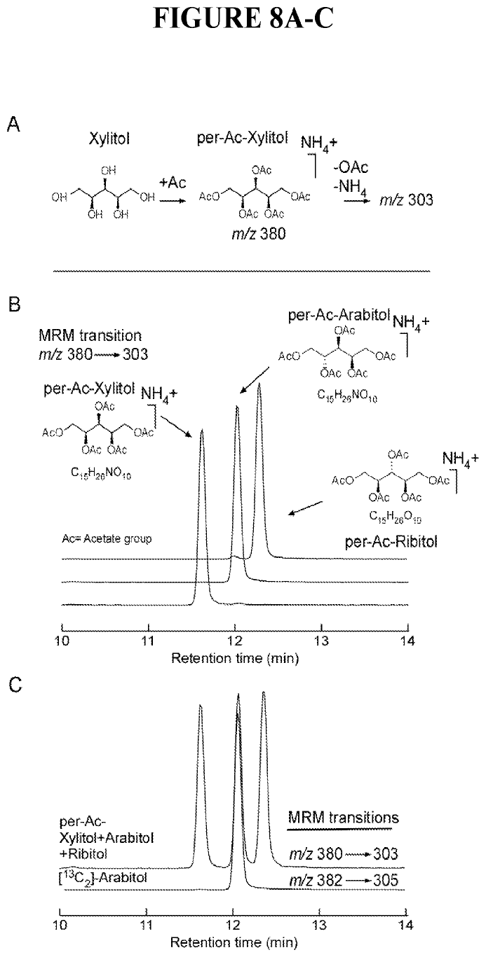

Stable-isotope-dilution LC-MS/MS was developed to specifically quantify xylitol in human and mouse plasma, urine and feces (fecal pellets were dissolved in water and supernatant subjected to LC-MS/MS). The chromatographic separation of xylitol from its structural isomers is shown in FIG. 8. Accuracy and precision of the method to quantify xylitol (separated from its structural isomers arabitol and rabitol) were assessed by a standard addition method 4 of pure synthetic standard into three different plasma pools. Accuracy was 2.4-2.8%, and intra- and inter-day coefficients of variation (CVs), were 1.2-8.9% and 10.9-11.5%, respectively. Urine creatinine was similarly analyzed by stable-isotope-dilution LC-MS/MS using D₃-creatinine as internal standard as previously described.⁴⁷ Erythritol was quantified by stable-isotope-dilution LC-MS/MS as previously described.¹⁹ Details for all assays can be found in the additional methods.

### Statistical Analysis

Continuous variables are summarized as median (interquartile range [IQR]), and categorical variables are presented as %. Difference between groups (e.g. tertiles or quartiles) were examined using Kruskal Wallis test for continuous variables and chisq (χ²) test for categorical variables. Kaplan-Meier analysis with Cox proportional-hazards regression was used for time-to-event analysis to determine hazard ratios (HR) and 95% confidence intervals (CI) for incident MACE. Adjustments included traditional cardiovascular risk factors (age, sex, diabetes mellitus, systolic blood pressure, low-density lipoprotein cholesterol levels, high-density lipoprotein cholesterol levels, triglyceride levels, smoking status) and high-sensitivity C-reactive protein (hsCRP). We confirmed that the proportionality hazards assumptions were met using the Schoenfeld residuals against the transformed time. Mann-Whitney U-test or Wilcoxon matched-pairs signed rank test were applied to continuous variables to examine differences between 2 groups. Kruskal-Wallis test with Dunn's post hoc test was used for pairwise multiple comparisons of ranked data. Two-way ANOVA with Šidák's multiple comparison post hoc test was used for multiple-group comparisons of aggregometry responses using different concentrations of agonists. For analysis of collagen-dependent platelet adhesion in whole blood, a two-way repeated measures ANOVA with Sidák's multiple comparison post hoc test was used. All reported measurements represent distinct samples. Data analyses were performed with R software (version 4.0.2) and GraphPad Prism software (version 9.1.2). A two-sided P<0.05 was considered statistically significant.

### Results

Untargeted Metabolomics Analyses in a Discovery Cohort Suggests Xylitol is Associated with Adverse Cardiovascular Outcomes

We have previously utilized untargeted metabolomics as a discovery platform to identify metabolites and pathways linked to residual CVD risks beyond traditional established risk factors.^{19,36,41,48} During the conduct of those untargeted mass spectrometry studies, we examined plasma from sequential stable subjects undergoing elective diagnostic cardiac evaluations (Discovery Cohort, n=1,157) and observed that numerous polyols, including a metabolite with presumed pre-derivatization elemental composition of C₅H₁₂O₅ and tentatively classified as xylitol, showed significant difference in level between those who experienced an incident (3 year) adverse cardiovascular event versus those who did not.¹⁹ In new studies, we performed further analyses of the raw untargeted metabolomics data from this Discovery Cohort (Table 1 shows baseline characteristics), this time utilizing a fragment ion more selective for xylitol as quantifying ion (Methods).

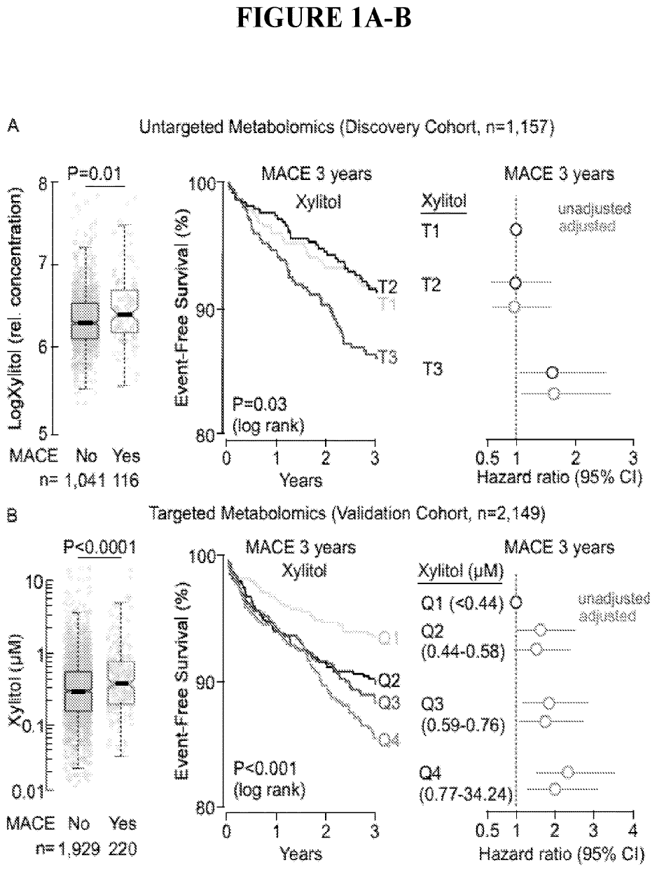

As shown in FIG. 1A, left, levels of the analyte tentatively annotated as xylitol showed enhanced relative abundance in subjects that experienced (versus not) a MACE during the ensuing 3 years of follow-up. Further examination showed subjects with increased levels of the analyte (tertile 3 (T3) versus either T1 or T2) displayed both poorer event-free survival in Kaplan-Meier survival analyses (FIG. 1A, middle), and significant increased risk for incident (3 year) MACE (T3 versus T1, hazard ratio (HR) [95% confidence interval (CI)], 1.63 [1.04-2.53], P<0.05) in Cox proportional hazards regression analyses, including following adjustments for traditional CVD risk factors and high-sensitivity C-reactive protein (hsCRP) (adjusted(a)HR [95% CI], 1.64 [1.05-2.56], P<0.05; FIG. 1A, right).

### Stable Isotope Dilution Tandem Mass Spectrometry Analyses of Xylitol in the Validation Cohort

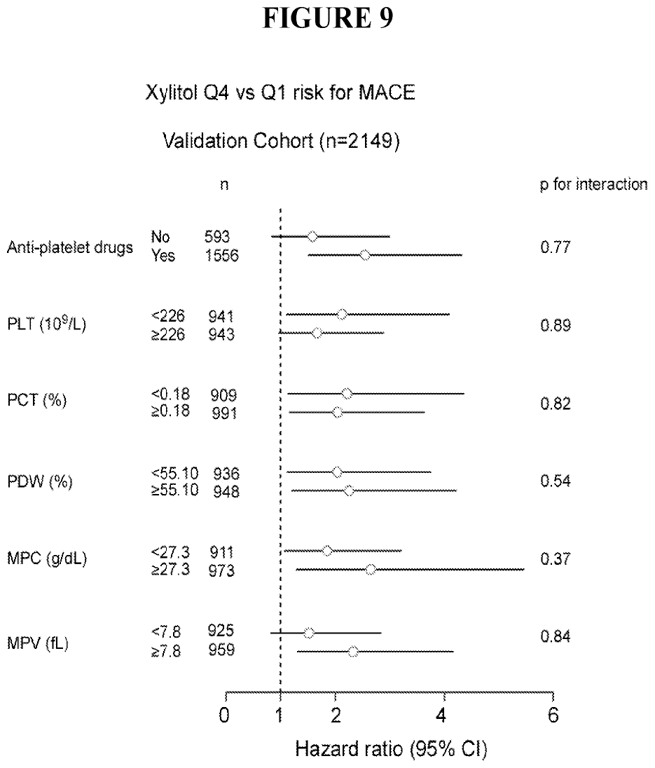

Untargeted metabolomics studies are both semi-quantitative and not designed to necessarily distinguish between structural isomers. For example, xylitol and its isomers arabitol and ribitol all share identical elemental composition (C₅H₁₂O₅), overall structure, and have similar physicochemical properties. The isomers also have similar mass spectrometry fragmentation patterns, and often co-chromatograph under routine separation protocols employed during untargeted metabolomics (Methods). We therefore developed a stable isotope dilution liquid tandem mass spectrometry (LC-MS/MS) assay with chromatography conditions that provided baseline separation of xylitol from its structural isomers (FIG. 8), and then applied the method to unequivocally quantify xylitol in an independent (non-overlapping) Validation Cohort (n=2,149) comprised of stable subjects undergoing elective diagnostic cardiac evaluation (Table 1). Higher plasma levels of xylitol were observed among subjects that experienced MACE (FIG. 1B, left). Kaplan-Meier analysis similarly revealed higher levels of circulating xylitol were associated with poorer event-free survival over the follow-up period (FIG. 1B, middle). In time to event Cox proportional hazards analysis, subjects with higher plasma xylitol levels had significantly increased risk of incident (3 year) MACE (HR [95% CI], 2.31 [1.52-3.52], P<0.0001), including following adjustments for traditional cardiovascular risk factors and hsCRP (aHR [95% CI], 1.98 [1.29-3.02], P=0.002, FIG. 1B, right). In parallel analyses, we looked at patient subgroups in the presence or absence of anti-platelet drugs and subgroups based on different platelet indices (FIG. 9). The association of xylitol with MACE risk was independent of all anti-platelet drug use, and all platelet markers examined, with the point estimate of the HR to the right of unity in all subgroups analyzed and no interaction observed (P for interaction in all subgroups >0.35).

### Examination of Postprandial Circulating Levels of Xylitol Following Ingestion of a Xylitol-Sweetened Drink

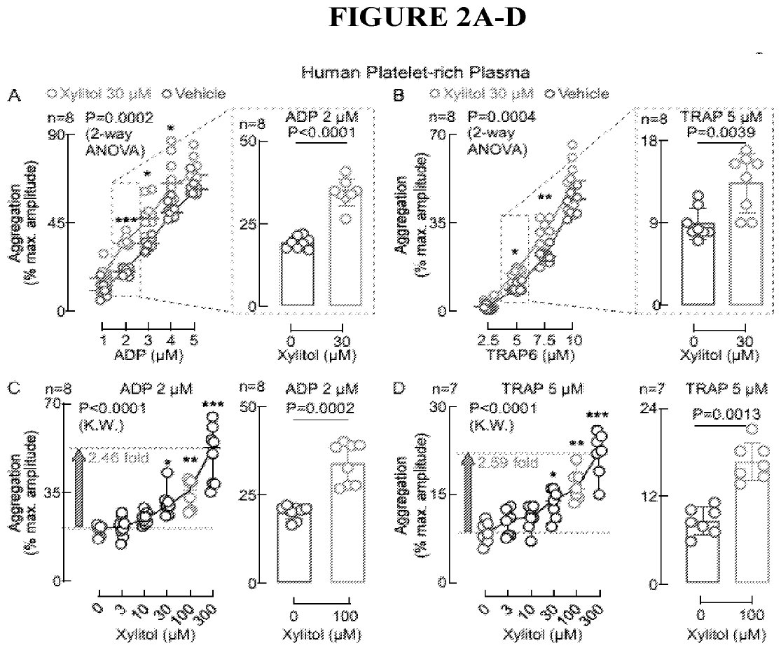

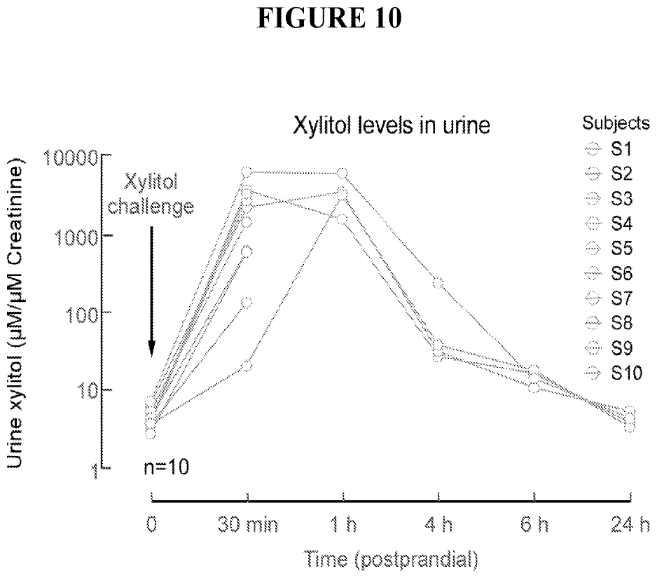

Plasma levels monitored in both the Discovery Cohort and the Validation Cohort were following overnight (>12 h) fast. Since “naturally sweetened” or “keto friendly” processed foods and beverages can contain relatively large amounts of xylitol, and thus might substantially increase circulating levels in the postprandial setting, before performing platelet function studies, we assessed how high circulating levels of xylitol reach following a commonly observed dietary exposure. Postprandial levels of xylitol were measured in heathy volunteers (n=10) following ingestion of a xylitol-sweetened (30 g) water, an exposure comparable to a pint of numerous xylitol-sweetened ice creams, a xylitol-sweetened bakery good, or several pieces of xylitol-sweetened candy (clinicaltrials.gov identifier NCT04731363, Methods). At baseline (following overnight fast), plasma levels of xylitol were low (median [inter quartile range (IQR)], 0.30[0.27-0.34]μM) and comparable to quartile 1 (Q1) values observed in the Validation cohort (FIG. 1B, right). However, 30 min following ingestion, 1000-fold increases in plasma levels were noted (median[IQR], 312[134-629]μM), with concurrent and subsequent excretion in the urine (FIGS. 2A and 10). Plasma xylitol levels returned to low-micromolar levels within 4 h to 6 h (median [IQR], 1.87[1.43-2.80]μM and 0.67[0.57-1.25]μM, respectively), with a plasma half-life of 12.6 (±3.9) min. The rapid rate of xylitol excretion observed in healthy volunteers with return to near baseline (fasting) levels within several hours following ingestion of a significant dietary exposure suggests that the plasma levels observed in our observational (Validation) cohort represent variations in endogenous production/levels, and not food intake.

### Physiological Levels of Xylitol Augment Platelet Responsiveness

The observed positive association between xylitol and incident thrombotic event risk (FIG. 1) suggested a potential impact of xylitol on platelet function. In initial studies, we assessed whether xylitol can impact platelet responsiveness using physiological concentrations of xylitol (i.e. at levels observed in overnight fasted subjects from our Validation Cohort, which is comprised of subjects with relatively preserved renal function, Table 1). Brief incubation of human platelet-rich plasma (PRP) recovered from healthy volunteers with a fixed physiological (Q4) level of xylitol (30 μM) showed no effect. However, when platelets were exposed to the same fixed xylitol concentration in the presence of sub-maximal levels of known agonists such as ADP or the thrombin receptor-activated peptide TRAP6, marked enhancement in stimulus-dependent platelet activation and aggregation were observed (FIGS. 2B and 2C). Employing a different study design, we next instead used a fixed submaximal level of each of the platelet agonists (ADP or TRAP6) and varying levels of pre-incubation with xylitol. Notably, a dose-dependent enhancement in the extent of platelet responsiveness was observed across both the physiological range of xylitol in fasting subjects in our clinical (Validation) cohort (e.g. 30 M), and extending to the postprandial levels observed in healthy volunteers following consumption of xylitol (e.g. we also examined 300 μM (FIGS. 2D and 2E), though postprandial levels of up to ~1000 M were observed, FIG. 2A). Collectively, these data show that within the range of fasting plasma (endogenous) levels of xylitol observed in our cohort, xylitol augments stimulus-dependent platelet responsiveness (i.e. shifts the dose response curve for known agonists to the left). Moreover, post-prandial levels of xylitol were observed to enhance platelet reactivity to an even greater extent.

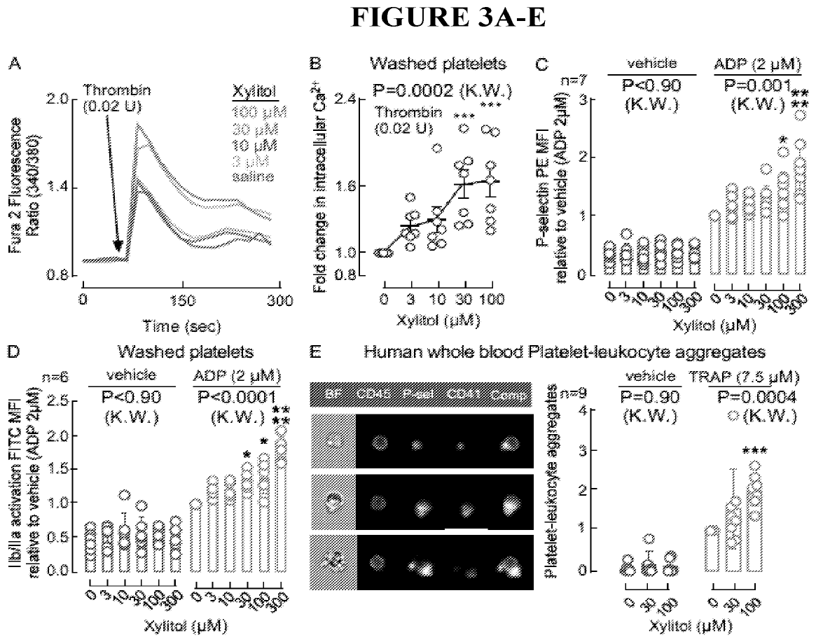

Platelet aggregation responses can involve factors beyond direct interaction with platelets. We therefore isolated washed human platelets from healthy volunteers to directly test whether xylitol interacts with the platelets and impacts agonist-induced intracellular cytosolic calcium release. Brief (15-30 min) pre-incubation of Fura 2-AM-loaded platelets with varying levels of xylitol dose-dependently enhanced submaximal (0.02 U) thrombin-evoked intracellular Ca²⁺ release in multiple different platelet preparations (FIGS. 3A and 3B). In parallel experiments, exposure of washed human platelets to physiological levels of xylitol caused dose-dependent enhancement in multiple examined platelet activation phenotypes including ADP-stimulated P-selectin surface expression and glycoprotein α2β3 (GP IIb/IIIa) activation (FIGS. 3C and 3D). Activated platelets bind to leukocytes—a process that leads to mutual changes in cellular effector functions and is associated with various CVD phenotypes.⁴⁹ We therefore also examined the effect of xylitol on platelet—leukocyte aggregates. For these studies we used imaging flow cytometry since it is optimized for cell-cell interaction and, in contrast to conventional flow cytometry, distinguishes between tethered platelets (genuine platelet-leukocyte aggregates) from coincidental, untethered platelets near leukocytes.⁵⁰ Following brief pre-incubation with a range of physiological levels of xylitol, whole blood from healthy volunteers showed a dose-dependent increase in TRAP6 stimulated platelet-leukocyte aggregates (FIG. 3E).

### Xylitol Enhances Platelet Clotting in Whole Blood and Thrombosis Potential In Vivo.

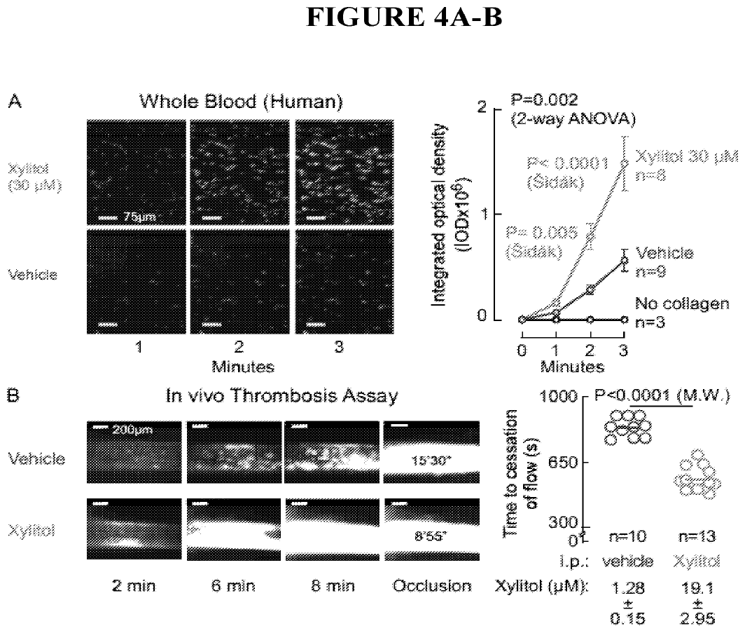

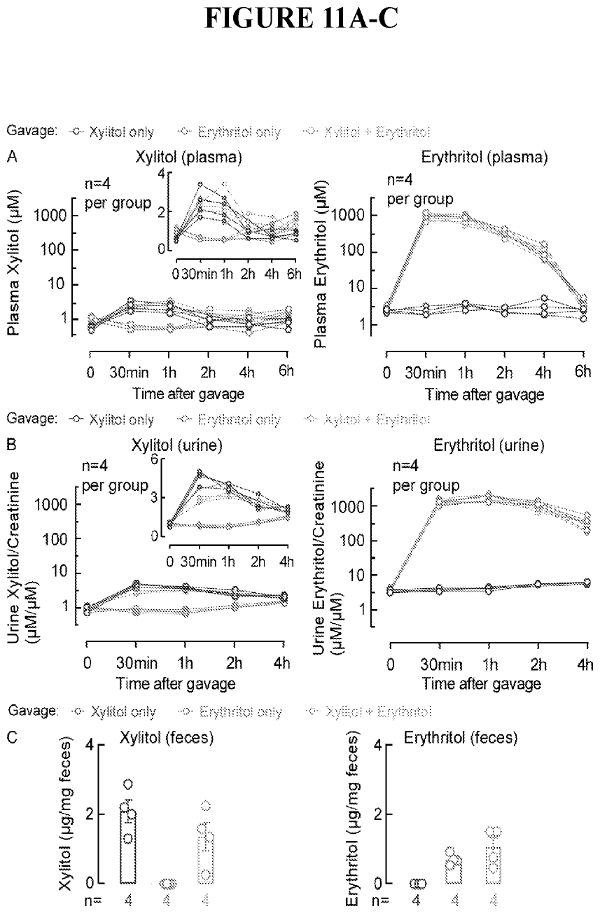

To investigate the effect of xylitol on the initial step in thrombus formation, platelet adhesion to a collagen surface, we examined platelet adhesion in whole blood under physiological shear flow using a microfluidics device.⁴⁰ Xylitol substantially accelerated the rate of collagen-dependent platelet adhesion and spreading under physiological shear flow (FIG. 4A). In additional studies, the impact of xylitol on in vivo thrombosis potential was assessed using a FeCl₃-induced carotid artery injury model.⁴⁴ In preliminary studies mice showed poor oral absorption of xylitol (in comparison to erythritol) in both feeding studies (when provided in drinking water or food), and in studies where the sugar alcohols were delivered by gastric gavage (FIG. 11). Thus, an i.p. injection model was performed to recapitulated physiological plasma levels of xylitol in mice that paralleled those observed in humans. Compared to vehicle (saline) control, mice with elevated plasma levels of xylitol exhibited both a marked increase in the rate of clot formation (FIG. 4B, left), and a significant reduction of the time to cessation of blood flow following arterial injury (FIG. 4B, right).

### Xylitol Dietary Challenge in Subjects Enhances Multiple Indices of Platelet Reactivity.

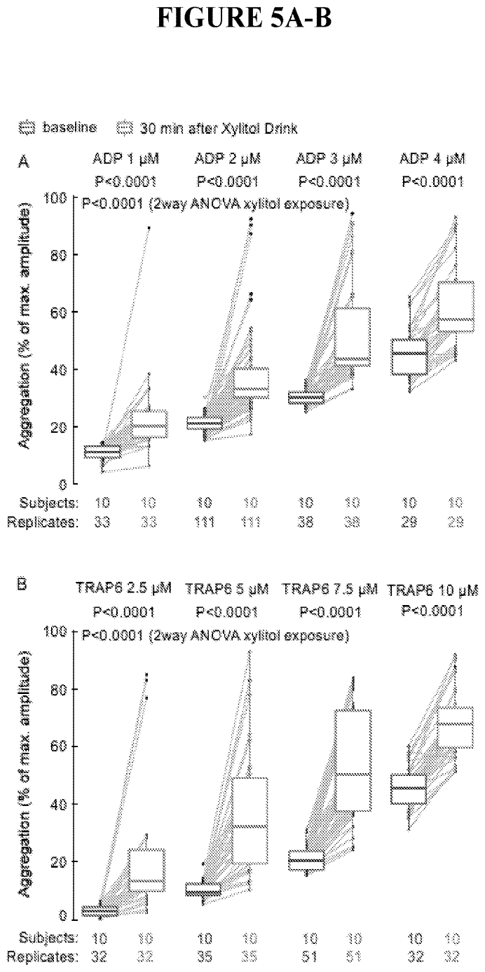

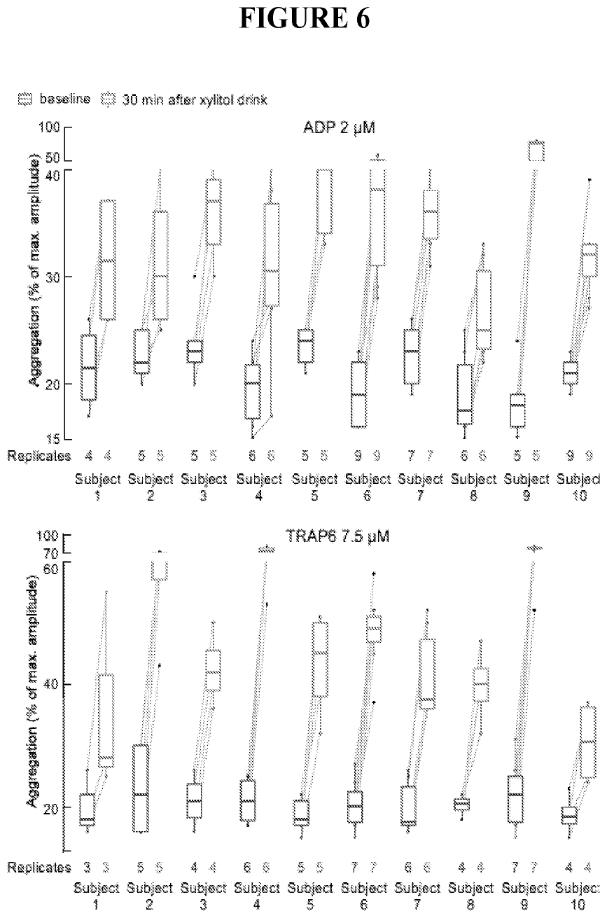

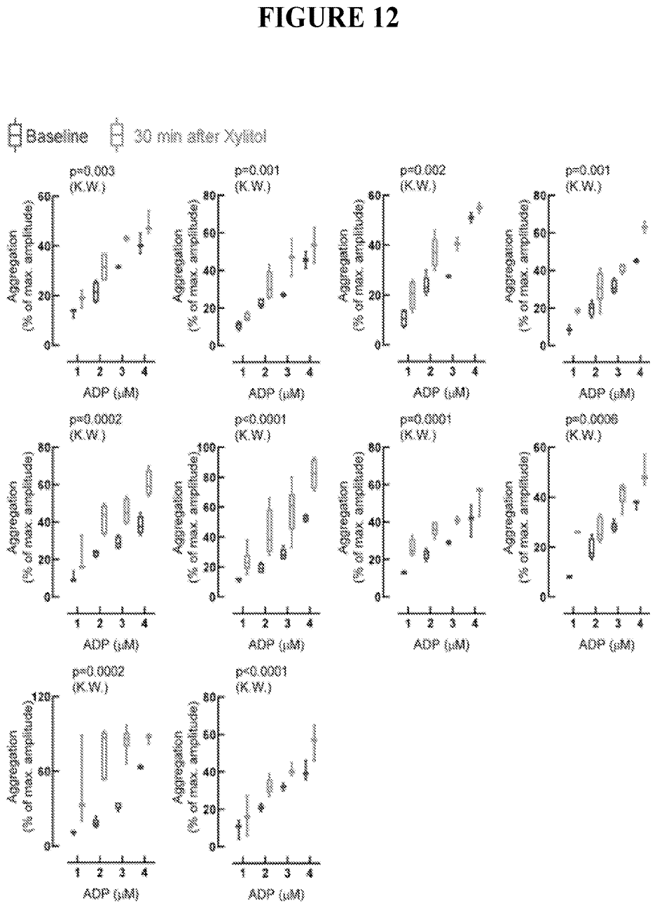

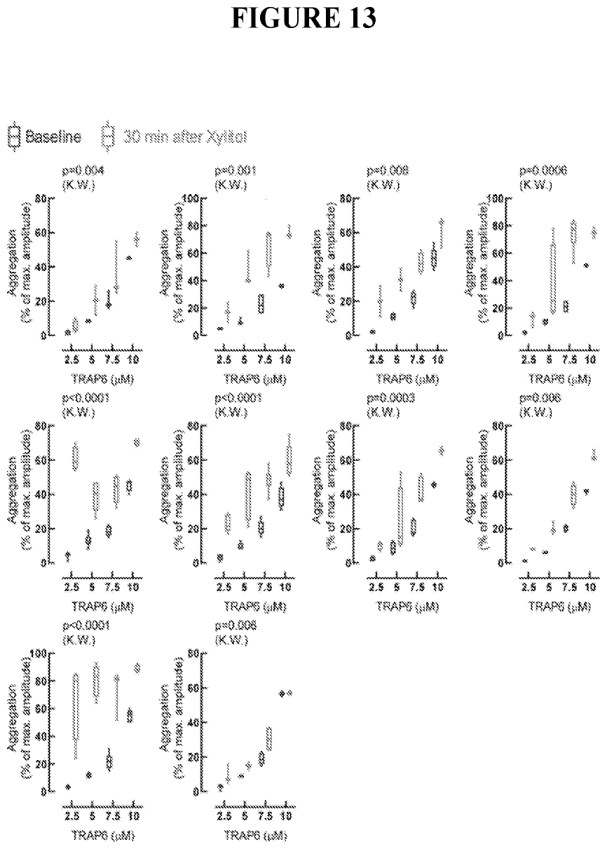

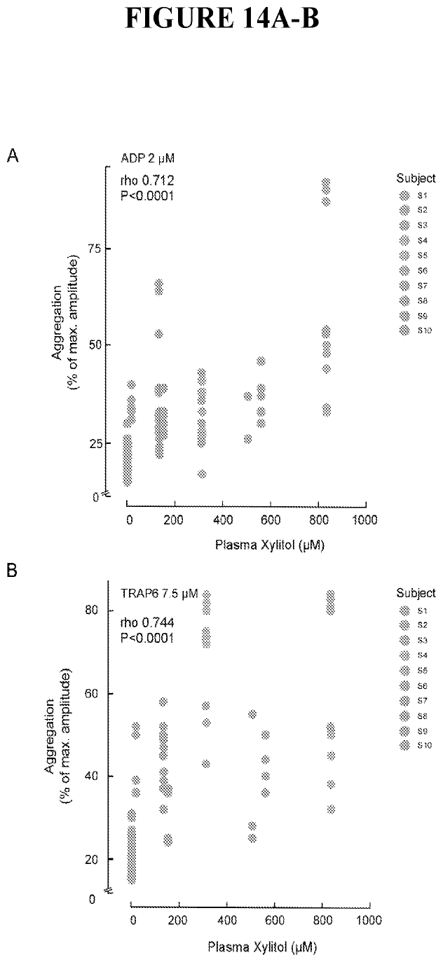

Since our studies with isolated washed human platelets, platelet rich plasma, whole blood, and murine in vivo thrombosis models, all suggested that xylitol can impact platelet responsiveness, we next sought to examine whether dietary exposure to xylitol impacted platelet phenotypes in humans following ingestion of xylitol-sweetened water. For these studies, we rapidly isolated PRP from healthy volunteers (n=10) before and 30 min after ingestion of xylitol (clinicaltrials.gov identifier NCT04731363, Methods) and assessed agonist-induced aggregation responses. As noted before, xylitol ingestion significantly increased postprandial (30 min) plasma levels (median [IQR], 312 [134-629]M). In parallel, platelet functional analyses revealed xylitol exposure provoked a substantial (multiple-fold) increase in aggregation responses to either ADP or TRAP6 (P<0.0001 at all doses examined, FIG. 5), in line with our in vitro studies using similar concentrations of xylitol (FIGS. 2C and D). When responses of individual subjects to submaximal agonist (ADP and TRAP6) concentrations were examined, a significant increase in platelet responsiveness was observed following xylitol ingestion in every subject (FIG. 6, FIGS. 12 and 13). Further, subjects showing the largest increases in platelet responsiveness post xylitol challenge also tended to have the highest postprandial xylitol levels, and a strong positive correlation was noted between postprandial xylitol levels and either ADP-induced or TRAP-induced aggregation responses (Spearman rho 0.71 and 0.74, respectively; P<0.0001 for each; FIG. 14).

Many studies, including data analyses from the National Health and Nutrition Examination Survey (NHANES), have confirmed a dramatic increase of low-calorie sweetener use over the past decades.⁵¹ Meanwhile, the consumption of sweeteners is likely underestimated because of lack of itemized listing of specific low-calorie sweeteners on labels in many reduced- and low-calorie processed foods, and the lack of disclosure requirements (e.g. for the quantity used) in policies for food labeling by both the FDA and EU.⁵² Remarkably, artificial sweeteners have even been detected in presumed “non-consumers” who were counseled extensively to avoid artificial sweetener exposure before they were enrolled into randomized clinical trials.⁵³ Moreover, the increases in artificial sweetener use have even reached levels where they are readily detected within ground water and waste effluent, where their detection and quantification has been recommended as “ideal chemical markers of domestic wastewater in groundwater”.⁵⁴ Although non-nutritive and low-calorie sweeteners have historically been regarded as safe by public agencies (e.g. EU⁴ and the FDA³), several cohort studies¹³⁻¹⁷, but not all^{55,56}, have linked ingestion of artificially sweetened foods with cardiometabolic adverse phenotypes. This recently prompted authorities, including the European Food Safety Authority (EFSA) and the World Health Organization, to re-evaluate exposure and toxicity of sweeteners, in particular with respect to potential long-term effects on health.^{57,58} In light of the substantial rise in the incorporation of artificial sweeteners into our food chain, and the present studies identifying both clinical and mechanistic links between xylitol and CVD risks and relevant phenotypes, further studies that assess their long-term cardiovascular safety seem warranted.

Polyol sweeteners like xylitol are difficult to quantify since they possess multiple structural isomers that differ only in the spatial orientation of the hydroxyl groups on the molecule. These difficulties, coupled with limited regulatory requirements for disclosure, have hampered their quantification in epidemiological studies to explore links between levels of sweeteners in blood and both metabolic and CVD risks. In the present example, an unambiguous link between plasma levels of xylitol and incident MACE risks was observed in our Validation Cohort, where xylitol was separated from its structural isomers and quantified using isotope dilution LC-MS/MS. Further, comparisons between xylitol levels that elicit increases in platelet reactivity and in vivo thrombosis potential suggest even at elevated plasma levels among overnight fasted subjects (e.g. Q4), significant increases in platelet responsiveness are observed. By performing human clinical intervention studies exploring the physiological effects of xylitol ingestion, a marked enhancement in multiple indices of platelet responsiveness was observed in every subject examined. Collectively, the body of evidence accrued, including both in vitro, in vivo (animal model), and xylitol human ingestion studies, argues for a direct effect of the low-calorie sweetener xylitol on platelet function and thrombosis potential in vivo.

Interestingly, during the conduct of these and prior studies, we note inter-species differences in the absorption and metabolism of different sugar alcohols, arguing for both the necessity of monitoring circulating levels of specific molecular species in studies (to confirm what is ingested is actually absorbed), as well as the need for human clinical investigations. For example, we found xylitol, like erythritol¹⁹, is readily absorbed following oral ingestion by humans, with plasma levels increasing over 1000-fold in the postprandial setting. However, our present studies also revealed that in mice, while erythritol is rapidly absorbed by oral route, xylitol is poorly absorbed, with the majority of the ingested sugar alcohol recovered in feces (FIG. 11). We therefore used intraperitoneal injection to elevate xylitol plasma levels in mice to the concentrations observed in humans, allowing for testing the effect of systemic exposures in rodent models analogous to those experienced in humans (Methods). Another example of inter-species differences in metabolism of sugar alcohols within mammals occurs with dogs. While xylitol does not induce glucose or insulin secretion in humans or rodents^{59,60}, it is highly toxic to dogs, where it provokes extreme insulin secretion,⁶¹ and numerous reports list the extensive use of xylitol in human processed foods as a warning for pet owners.⁶²

Despite the clear evidence for xylitol inducing a direct physiological effect on isolated human platelets, the molecular participants involved (i.e. receptor) transmitting the effect on platelet function remains unknown. Indeed, little is known about how polyol sweeteners are recognized by cells. In the present studies, when added at submaximal levels, xylitol enhanced stimulus induced platelet aggregation responses and in vivo thrombosis formation. Collectively, when coupled with recent observations with erythritol¹⁹, our data suggest that xylitol and erythritol act on converging pathways to enhance platelet responsiveness and in vivo thrombosis potential. They thus argue for a potential adverse class effect of sugar alcohol sweeteners, a finding that is highly relevant for both the processed food industry, and the diets of the more vulnerable subjects most likely to consume multiple artificial sweeteners (diabetics, obese, those with CVD).

One topic that deserves further discussion is the relationship in the large-scale clinical observational studies performed associating plasma levels of xylitol with incident risks of MACE, and dietary exposures to xylitol. The present studies, we believe, argue that the observed associations noted in the Discovery and Validation Cohorts reflect endogenous xylitol levels (steady state between endogenous production and excretion), and not recent dietary exposure. First, while a limitation of the present example is the lack of any information on xylitol dietary exposure in the clinical observational cohorts, given the speed with which we observe xylitol is excreted (i.e. postprandial plasma levels in healthy volunteers return to near baseline levels within 6 hours; FIG. 2A), the fasting (>12 h) plasma levels monitored in the Discovery and Validation Cohorts likely represent endogenous levels of xylitol production. Second, the enrollment of subjects in the Validation Cohort largely predate (2004-2011) the more recent increases in dietary exposure to xylitol in processed foods. We also note that it is across variations in endogenous levels (measured in overnight (>12 h) fasting samples) that our initial clinical observational studies observe heightened MACE risks in subjects with higher xylitol levels (e.g. Q4 vs Q1). Xylitol is endogenously produced within cells where it serves as an intermediate of the glucuronic acid pathway—an alternative route of glucose utilization that provides biosynthetic precursors and involves detoxification through glucuronidation with an estimated endogenous production of 15 g daily in subjects.^{31,63,64} As far as we are aware, the glucuronic acid pathway has not yet been linked with CVD pathogenesis. However, we note that in recent studies glucuronic acid levels were reported to be associated with health span and longevity in both population-based cohort studies, and in mice.⁶⁵ Yet other studies have linked glucuronic acid pathway intermediates with cell migration and metastasis.⁶⁶ Finally, we also think it important to mention that throughout the evolution of humans, Homo sapiens have not experienced millimolar levels of xylitol in plasma—that is, until its introduction into our food chain within the past decade or so. The present studies show, however, that even at plasma xylitol levels as low as 19 μM in animal models of thrombosis (FIG. 4B), well within the 4th quartile of our fasting plasma levels from a cohort (Validation) of subjects with largely preserved renal function, we observe significant heightened thrombosis potential in vivo. Based on our additional human intervention study data (FIGS. 2, 5 and 6), much higher postprandial levels of xylitol (driven by consumption of dietary xylitol from artificially sweetened processed foods) can be superimposed on endogenous levels, substantially further enhancing thrombotic risk (especially in the post-prandial setting).

### Additional Methods

### Aggregometry Studies in Platelet-Rich Plasma

Platelet rich plasma (PRP) was isolated as previously described.⁵ Whole blood was drawn from healthy donors and anticoagulated with sodium citrate (0.109 M). PRP was prepared by centrifuging at 100×g for 10 min at 22° C. Platelet poor plasma (PPP) was obtained by additional centrifugation of the remaining blood samples at 11,000×g for 2 min. Next, platelets were counted with a hemocytometer and the platelet concentration adjusted to 2×10⁸/mL using PPP. PRPs were then pre-incubated with xylitol (Catalogue #X3375-5G, Sigma, St. Louis, MO, USA) at indicated concentrations or vehicle (saline) for 30 min at 22° C. After exposure to xylitol or vehicle, PRPs were maintained in suspension with constant stirring (600 rpm) at 37° C. Subsequently, platelet aggregation was induced by adding the agonists ADP (up to 5 μM, Catalogue #384, Chronolog, Havertown, PA, US) or TRAP6 (TFLLR-NH2, up to 10 μM, Catalogue #464, Tocris, Bristol, UK). For all platelet aggregometry experiments, blood was always processed within approx. 30 minutes of collection, and isolated platelets were used within 120 minutes of isolation. When used, pairs of vehicle vs. xylitol incubated PRP were always incubated and analyzed together (beginning, middle, end of platelet prep use) and after 120 minutes post isolation, PRPs were not used anymore.

### Intracellular Calcium Measurements

To isolate washed platelets for intracellular Ca²⁺ measurements, PRP was prepared as described above. Next, 100 nM prostaglandin E1 (PGE-1, Catalogue #P5512, Sigma, St. Louis, MO, USA) was added to the PRP and centrifuged at 500×g for 20 min at 22° C. as previously described.⁶ After a gentle wash in a modified phosphate buffer saline (NaCl (137 mM), KCl (2.7 mM), Na₂HPO₄ (12 mM), MgCl₂ (1 mM), and glucose (5.5 mM), pH 7.4) with PGE-1 (100 nM), the platelet pellet was spun again at 500×g for 20 min. The platelet pellet was re-suspended in modified Hank's buffered salt solution (HBSS-BSA-glucose; NaCl (0.137 M), KCl (5.4 mM), Na₂HPO₄ (0.25 mM KH₂PO₄ (0.44 mM), CaCl₂ (1.3 mM), MgSO₄ (1.0 mM), NaHCO₃ (4.2 mM), glucose (5 mM) and BSA (0.1%)) with 100 nM PGE-1 and incubated with Fura 2-AM (1 mM) at 22° C. After 30 min of incubation, an additional centrifugation at 500×g for 30 min was performed to remove excess Fura 2-AM. After resuspension in modified Hank's buffered salt solution the platelet pellet was incubated with xylitol at the indicated concentrations or vehicle for 30 min at 22° C. Intracellular Ca²⁺ release was initiated by submaximal concentration of thrombin (0.02 U/mL) and changes in intracellular Ca²⁺ monitored via Fura 2-AM fluorescence using 340/380 nm dual-wavelength excitation and an emission of 510 nm.

### Platelet Flow Cytometry Assay

Antibody staining of washed platelets for flow cytometry was performed as described previously.⁶ Whole blood was collected and anticoagulated with sodium citrate (0.109 M). PRP was generated by centrifuging at 100×g for 10 min at 22° C. and re-suspended in modified Hank's buffered salt solution containing PGE-1 (100 nM). Washed platelets were isolated by centrifugation at 500×g for 10 min and re-suspended in modified Hank's buffered salt solution without PGE1. The final platelet count was adjusted to 2×10⁸ platelets/mL and the suspension then pre-incubated with xylitol (at indicated concentrations) or vehicle for 30 min at 22° C. Where indicated, the platelets were stimulated with ADP (2 μM) for 10 min. The platelets were then incubated with a PE-conjugated anti-P-selectin (CD62P-PE, Catalogue #555524, BD PharMingen, San Diego, CA, USA) or Fluorescein isothiocyanate(FITC)-conjugated PAC1 (binds only to active conformation of GP IIb/IIIa, Catalogue #340507, BD PharMingen, San Diego, CA, USA) or isotype control antibody (PE IgG isotype control, Catalogue #555749 or FITC IgM Isotype control, Catalogue #555583, BD PharMingen, San Diego, CA, USA) in the dark for 20 min. The washed platelets were subsequently fixed with 2% paraformaldehyde. Data was acquired using a flow cytometer (FACS LSR Fortessa, BD Biosciences, Franklin Lakes, New Jersey, USA) with twenty thousand (20,000) events recorded. We used FACSDiva Software (v.9.0) (BD Biosciences) for the data analysis. Platelets were gated to exclude doublets and the raw mean fluorescent intensity (MFI) of either P-selectin (CD62P) or PAC-1 was quantified.

### Imaging Flow Cytometry in Whole Blood

Whole blood collected from consented healthy volunteers was incubated with xylitol (30 or 100 μM) or vehicle for 30 min. Where indicated, the samples were then stimulated with 7.5 μM TRAP6 for 10 min. Blood samples were then incubated with anti-CD45 AF488 (Catalogue #FAB1430G, R&D Systems, MN, USA), anti-CD41 AF647 (Catalogue #303726, Biolegend, San Diego, CA, USA) and anti-CD62P-PE (Catalogue #555524, BD PharMingen, San Diego, CA, USA) for 20 min at room temperature in the dark. After gentle red blood cell lysis using VersaLyse Lysing solution (Catalogue #A09777, Beckman Coulter, Brea, California, USA), cells were gently spun down and resuspended in HBSS-BSA. Platelet leucocyte aggregates were measured on an Amnis ImageStreanX MK II two-camera system (Luminex, Austin, TX, USA). Data were analyzed using Amnis IDEAS software (Luminex, Austin, TX, USA).

IDEAS analysis was performed as follows. Gating out of focus events using Gradient RMS in the brightfield image. Gating on CD45+ events using the Intensity and Max Pixel features. Elimination of false positive CD45 events and selection of only true CD45+ cells using Intensity vs. Area of CD45+. Elimination of CD45+ doublets and aggregates using Area vs. Aspect Ratio. Gating on CD45+CD41+ events using the Intensity and Max Pixel features. Starting from CD45+ cells, Intensity vs. Max Pixel of CD41 was plotted and positive events were selected. Gating on CD45+CD62P+ positive events using the Intensity and Max Pixel features. Creation of a combined CD45+CD62P+ and CD41+ positive population using Boolean logic. Determination of whether CD62P and CD41 signals are overlapping, touching, or non-touching from the Similarity vs. Delta Centroid XY features. The number of CD45+, CD62P+ and CD41+ events/all CD45+ cells of each sample was used for the analysis.

### Whole Blood In Vitro Thrombosis Assay

Shear flow experiments of whole blood were performed with a Cellix Microfluidics System (Cellix, Dublin, Ireland) as previously described.⁶⁻⁸ Before use, the micro channels of a Vena8 Fluoro+ biochip were coated with type 1 collagen (15 μL; 50 μg/mL) and each channel of the biochip then washed with PBS using the Mirus Nanopump. Images were taken with an HC Plan Apo 20X/0.7NA lens on a Leica DMI6000 inverted microscope equipped with an environmental chamber and a Hamamatsu ImagEM cooled CCD camera. Whole blood was incubated with an Alexa Fluor®488-conjugated anti-human CD42b antibody (catalogue #303914, Biolegend, San Diego, CA, USA) to label platelets and exposed to xylitol (30 M) or vehicle (saline) for 30 min at 22° C. The blood was then perfused over chips coated with (or without) immobilized type 1 collagen at a physiological shear rate (60 dynes/cm²) using a multi-channel microfluidic device over the course of 3 min. Images of platelets adhering to the collagen matrix were recorded every 5 s. When the experiment was finished, the biochip was washed with PBS at 20 dynes/cm² and five images were captured along the length of the channel. The extent of platelet activation and adhesion to the collagen matrix was quantified using computer assisted tomographic analyses as previously described.⁵ Briefly, images of CD42b stained platelet thrombi were quantified using Image Pro plus software (Media Cybernetics, Rockville, Maryland, USA). The intensity threshold was chosen to select for specific staining and the integrated optical density (IOD, Area×Intensity) quantified.

### Carotid Artery FeCl₃ Injury Model

The FeCl₃-induced carotid artery injury model was performed as previously described.⁹ BL/6J mice, both male and female, 12-14 weeks of age, were i.p. injected with vehicle (saline) or xylitol (25 mg/kg). The animals were anesthetized with 100 mg/kg ketamine+10 mg/kg xylazine and rhodamine 6G (100 μL; 0.5 mg/mL, catalogue #252433, Sigma, St. Louis, MO, USA) was injected into the right jugular vein for platelet labeling. The left carotid artery was then exposed, and an injury induced using a 1 mm² Whatman filter paper containing 10% FeCl₃ (Catalogue #157740, Sigma, St. Louis, MO, USA) for 1 min. Thrombus formation in real time was monitored using intravital fluorescence microscopy equipped with video recording. Time to cessation of blood flow through thrombus formation for all experiments was determined by visual inspection by two independent investigators and animals were immediately euthanized after data acquisition.

### Untargeted GC-MS Analysis of Human Plasma

Subject plasma samples were derivatized and run on GC-MS analyses as previously described.¹⁰ Raw data files were processed using the metabolomics BinBase database.¹¹ All database entries in BinBase were matched against UC Davis metabolomics center's mass spectral library. The fragment ion m/z 217 was used for quantifying the predicted derivatized xylitol analyte.

### Targeted Mass Spectrometry Analyses

Stable-isotope-dilution LC-MS/MS was developed to specifically quantify xylitol in human and mouse plasma, urine and feces (fecal pellets were dissolved in water and supernatant subjected to LC-MS/MS). Ice cold methanol (800 μL) and internal standard (¹³C₂-arabitol) were added to the plasma samples (20 μL) or urine (20 μL) previously diluted in MQ water, followed by vortexing and centrifuging (21,000×g; 4° C. for 15 min). The clear supernatant (800 μL) was transferred into a clean, labeled glass tubes (Borosilicate glass 12×75 mm) and dried in a speed vacuum concentrator (Speed vac plus, SC210, Thermo Sevant). Acetylation was used since xylitol is poorly ionizable in order to achieve better retention on the reverse phase column and separation from other structural isomers as well as better ionization to achieve better sensitivity. The dry residue was reconstituted in acetic anhydride (100 μL) and 4-Dimethylaminopyridine (DMAP) in pyridine (100 μl; 1 mg/mL), sealed with safety caps, vortexed and heated (45 min at 80° C.) followed with drying under nitrogen. Dried residues were dissolved in HCl in water (0.1 M; 0.5 mL) and extracted with ethylacetate (2.0 mL). Ethylacetate layer was transferred into a clean glass tube (Borosilicate glass 12×75 mm) and dried under nitrogen. The dry residue was reconstituted in ammonium formate in a mixture of methanol:water (100 μL; 50:50 v/v with 10 mM ammonium formate), tubes were vortexed and liquid was transferred to glass vials with micro-insets and caped. LC-MS/MS analysis was performed on a chromatographic system consisting of two Shimadzu LC-30 AD pumps (Nexcra X2), a CTO 20AC oven operating at 30° C., and a SIL-30 AC-MP autosampler in tandem with a triple quadruple mass spectrometer (8050 series, Shimadzu Scientific Instruments, Inc., Columbia, MD, USA). For chromatographic separation, a Kinetex C18 column (50 mm×2.1 mm; 2.6 μm) (Cat #00B-4462-AN, Phenomenex, Torrance, CA) was used. Solvent A (10 mM ammonium formate and 0.1% formic acid in water) and B (10 mM ammonium formate and 0.1% formic acid in acetonitrile:water 95:5 (v/v)) were run using the following gradient: 0.0 min (0% B); 0.0-11.0 min (25% B); 11.0-14.0 min (25% B→30% B); 14.0-17.0 min (30% B→35% B); 17.0-19.0 min (35% B); 19.0-22.0 min (35% B→40% B); 22.0-22.5 min (100%); 22.5-25 min (100% B); 25.0-26.0 min (100% B→0% B); 26.0-28.0 min (0% B) with flow rate of 0.35 mL/min and an injection volume of 1 μL. Electrospray ionization in the positive mode was used with multiple reaction monitoring (MRM) for detection of endogenous and stable isotope labeled internal standards. The following transitions were used: m/z 380 [M⁺NH₄]⁺ 4303 for xylitol, arabitol and ribitol and m/z 382 [M+NH₄]⁺→305 for [¹³C₂]-arabitol. The following ion source parameters were applied: nebulizing gas flow, 3 L/min; heating gas flow, 10 L/min; interface temperature, 300° C.; desolvation line temperature, 250° C.; heat block temperature, 400° C.; and drying gas flow, 10 L/min. Accuracy and precision of the method to quantify xylitol (separated from its structural isomers arabitol and ribitol) were assessed by a standard addition method of pure synthetic standard into different plasma pools. The accuracy of xylitol measurement was determined by the method of standard additions¹² using three different plasma pools, and was 2.4-2.8%. Intra- and inter-day coefficients of variation (CVs), measured by multiple analysis of three different control samples, were 1.2-8.9% and 10.9-11.5%, respectively. Freeze/thaw stability was determined over the five cycles and it was less than 7% for xylitol. The limit of quantification (LOQ) was defined (by convention) as the level at which xylitol was measured within the matrix (plasma) with a peak having signal-to-noise-ratio of 10:1. Limit of detection (LOD) was determined to be the lowest concentration of xylitol in the sample with a signal-to-noise ratio ≥3. LOD and LOQ for xylitol quantification were 0.019 and 0.063 μM; respectively. Three quality control samples were run with each batch of samples at the beginning, middle and the end of each batch and inter-batch variations expressed as CV were less than 12%. Further, we did not observe any matrix effect on metabolites retention times when compared to pure standards dissolved in water. Data were collected and analyzed by LabSolution 5.91 software (Shimadzu). The chromatographic separation of xylitol from its structural isomers is shown in FIG. 8. Urine creatinine was similarly analyzed by stable-isotope-dilution LC-MS/MS using D₃-creatinine as internal standard as previously described.¹³ Erythritol was quantified as previously described.¹⁰

### References for Additional Methods

1. Tang, W. H., et al. Intestinal microbial metabolism of phosphatidylcholine and cardiovascular risk. N Engl J Med 368, 1575-1584 (2013).
2. Wang, Z., et al. Gut flora metabolism of phosphatidylcholine promotes cardiovascular disease. Nature 472, 57-63 (2011).
3. Inker, L. A., et al. New Creatinine- and Cystatin C-Based Equations to Estimate GFR without Race. N Engl J Med 385, 1737-1749 (2021).
4. STROBE Statement—checklist of items that should be included in reports of observational studies1 (© STROBE Initiative). International Journal of Public Health 53, 3-4 (2008).
5. Zhu, W., et al. Gut Microbial Metabolite TMAO Enhances Platelet Hyperreactivity and Thrombosis Risk. Cell 165, 111-124 (2016).
6. Nemet, I., et al. A Cardiovascular Disease-Linked Gut Microbial Metabolite Acts via Adrenergic Receptors. Cell 180, 862-877.e822 (2020).
7. Gupta, N., Li, W. & McIntyre, T. M. Deubiquitinases Modulate Platelet Proteome Ubiquitination, Aggregation, and Thrombosis. Arterioscler Thromb Vase Biol 35, 2657-2666 (2015).
8. Scavone, M., et al. Platelet Adhesion and Thrombus Formation in Microchannels: The Effect of Assay-Dependent Variables. Int J Mol Sci 21, 750 (2020).
9. Witkowski, M., et al. Vascular endothelial Tissue Factor contributes to trimethylamine N-oxide-enhanced arterial thrombosis. Cardiovasc Res (2021).
10. Witkowski, M., et al. The artificial sweetener erythritol and cardiovascular event risk. Nat Med 29, 710-718 (2023).
11. Fiehn, O., Wohlgemuth, G. & Scholz, M. Setup and Annotation of Metabolomic Experiments by Integrating Biological and Mass Spectrometric Metadata. in Data Integration in the Life Sciences (eds. Ludascher, B. & Raschid, L.) 224-239 (Springer Berlin Heidelberg, Berlin, Heidelberg, 2005).
12. Whitmire, M. L., et al. LC-MS/MS Bioanalysis Method Development, Validation, and Sample Analysis: Points to Consider When Conducting Nonclinical and Clinical Studies in Accordance with Current Regulatory Guidances. Journal of analytical and bioanalytical techniques 2011(2011).
13. Wang, Z., et al. Impact of chronic dietary red meat, white meat, or non-meat protein on trimethylamine N-oxide metabolism and renal excretion in healthy men and women. Eur Heart J 40, 583-594 (2019).

### References for Example 1

1. Abarca-Gómez, L., et al. Worldwide trends in body-mass index, underweight, overweight, and obesity from 1975 to 2016: a pooled analysis of 2416 population-based measurement studies in 128-9 million children, adolescents, and adults. The Lancet 390, 2627-2642 (2017).
2. Hu, F. B. Resolved: there is sufficient scientific evidence that decreasing sugar-sweetened beverage consumption will reduce the prevalence of obesity and obesity-related diseases. Obes Rev 14, 606-619 (2013).
3. Roberts, A. The safety and regulatory process for low calorie sweeteners in the United States. Physiol Behav 164, 439-444 (2016).
4. Mortensen, A. Sweeteners permitted in the European Union: safety aspects. Scandinavian Journal of Food and Nutrition 50, 104-116 (2006).
5. World Health Organization. https://www.who.int/publications/i/item/9789240073616 (5/25/2023).
6. Sylvetsky, A. C., Welsh, J. A., Brown, R. J. & Vos, M. B. Low-calorie sweetener consumption is increasing in the United States. The American Journal of Clinical Nutrition 96, 640-646 (2012).
7. Gardner, C., et al. Nonnutritive sweeteners: current use and health perspectives: a scientific statement from the American Heart Association and the American Diabetes Association. Circulation 126, 509-519 (2012).
8. Association, B.D. Policy statement—The Use of Artificial Sweeteners.
9. Australian Diabetes Society. The Australian Obesity Management Algorithm. https://diabetessociety.com.au/documents/ObesityManagementAlgorithm18.10.2016FINAL.pdf
10. Diabetes Canada Clinical Practice Guidelines Expert Committee. Diabetes Canada 2018 Clinical Practice Guidelines for the Prevention and Management of Diabetes in Canada. Can J Diabetes. 2018; 42(Suppl 1):S1-S325, (2018).
11. Johnson, R. K., et al. Low-Calorie Sweetened Beverages and Cardiometabolic Health: A Science Advisory From the American Heart Association. Circulation 138, e126-e140 (2018).
12. Lohner, S., Toews, I. & Meerpohl, J. J. Health outcomes of non-nutritive sweeteners: analysis of the research landscape. Nutr J 16, 55 (2017).
13. Ruanpeng, D., Thongprayoon, C., Cheungpasitporn, W. & Harindhanavudhi, T. Sugar and artificially sweetened beverages linked to obesity: a systematic review and meta-analysis. Qjm 110, 513-520 (2017).
14. Imamura, F., et al. Consumption of sugar sweetened beverages, artificially sweetened beverages, and fruit juice and incidence of type 2 diabetes: systematic review, meta-analysis, and estimation of population attributable fraction. BMJ: British Medical Journal 351, h3576 (2015).
15. Chazelas, E., et al. Sugary Drinks, Artificially-Sweetened Beverages, and Cardiovascular Disease in the NutriNet-Santé Cohort. Journal of the American College of Cardiology 76, 2175-2177 (2020).
16. Malik, V. S., et al. Long-Term Consumption of Sugar-Sweetened and Artificially Sweetened Beverages and Risk of Mortality in US Adults. Circulation 139, 2113-2125 (2019).
17. Mullee, A., et al. Association Between Soft Drink Consumption and Mortality in 10 European Countries. JAMA Internal Medicine 179, 1479-1490 (2019).
18. Bhagavathula, A. S., Rahmani, J., Vidyasagar, K., Tesfaye, W. & Khubchandani, J. Sweetened beverage consumption and risk of cardiovascular mortality: A systematic review and meta-analysis. Diabetes Metab Syndr 16, 102462 (2022).
19. Witkowski, M., et al. The artificial sweetener erythritol and cardiovascular event risk. Nat Med 29, 710-718 (2023).
20. Toews, I., Lohner, S., Kiillenberg de Gaudry, D., Sommer, H. & Meerpohl, J. J. Association between intake of non-sugar sweeteners and health outcomes: systematic review and meta-analyses of randomised and non-randomised controlled trials and observational studies. Bmj 364, k4718 (2019).
21. Azad, M. B., et al. Nonnutritive sweeteners and cardiometabolic health: a systematic review and meta-analysis of randomized controlled trials and prospective cohort studies. Cmaj 189, E929-e939 (2017).
22. Suez, J., et al. Personalized microbiome-driven effects of non-nutritive sweeteners on human glucose tolerance. Cell 185, 3307-3328.e3319 (2022).
23. Romo-Romo, A., Aguilar-Salinas, C. A., Brito-Córdova, G. X., Gómez-Díaz, R. A. & Almeda-Valdes, P. Sucralose decreases insulin sensitivity in healthy subjects: a randomized controlled trial. Am J Clin Nutr 108, 485-491 (2018).
24. Miller, P. E. & Perez, V. Low-calorie sweeteners and body weight and composition: a meta-analysis of randomized controlled trials and prospective cohort studies. Am J Clin Nutr 100, 765-777 (2014).
25. McGlynn, N. D., et al. Association of Low- and No-Calorie Sweetened Beverages as a Replacement for Sugar-Sweetened Beverages With Body Weight and Cardiometabolic Risk: A Systematic Review and Meta-analysis. JAMA Netw Open 5, e222092 (2022).
26. Sylvetsky, A. C., Blau, J. E. & Rother, K. I. Understanding the metabolic and health effects of low-calorie sweeteners: methodological considerations and implications for future research. Rev Endocr Metab Disord 17, 187-194 (2016).
27. Washuttl, J., Riederer, P. & Bancher, E. A qualitative and quantitative study of sugar-alcohols in several foods. Journal of Food Science 38, 1262-1263 (1973).
28. Scheinin, A., Makinen, K. K., Tammisalo, E. & Rekola, M. Turku sugar studies XVIII. Incidence of dental caries in relation to 1-year consumption of xylitol chewing gum. Acta Odontol Scand 33, 269-278 (1975).
29. Grembecka, M. Sugar alcohols-their role in the modern world of sweeteners: a review. European Food Research and Technology 241, 1-14 (2015).
30. Livesey, G. Health potential of polyols as sugar replacers, with emphasis on low glycaemic properties. Nutr Res Rev 16, 163-191 (2003).
31. Wamelink, M. M., Struys, E. A. & Jakobs, C. The biochemistry, metabolism and inherited defects of the pentose phosphate pathway: a review. J Inherit Metab Dis 31, 703-717 (2008).
32. Milgrom, P., Rothen, M. & Milgrom, L. Developing Public Health Interventions with Xylitol for the US and US-Associated Territories and States. Suom Hammaslaakarilehti 13, 2-11 (2006).
33. Mussatto, S. I. Application of Xylitol in Food Formulations and Benefits for Health. in D-Xylitol: Fermentative Production, Application and Commercialization (eds. da Silva, S. S. & Chandel, A. K.) 309-323 (Springer Berlin Heidelberg, Berlin, Heidelberg, 2012).
34. Wölnerhanssen, B. K., Meyer-Gerspach, A. C., Beglinger, C. & Islam, M. S. Metabolic effects of the natural sweeteners xylitol and erythritol: A comprehensive review. Crit Rev Food Sci Nutr 60, 1986-1998 (2020).
35. Tang, W. H., et al. Intestinal microbial metabolism of phosphatidylcholine and cardiovascular risk. N Engl J Med 368, 1575-1584 (2013).
36. Wang, Z., et al. Gut flora metabolism of phosphatidylcholine promotes cardiovascular disease. Nature 472, 57-63 (2011).
37. Inker, L. A., et al. New Creatinine- and Cystatin C-Based Equations to Estimate GFR without Race. N Engl J Med 385, 1737-1749 (2021).
38. Brennan, M. L., et al. Comprehensive peroxidase-based hematologic profiling for the prediction of 1-year myocardial infarction and death. Circulation 122, 70-79 (2010).
39. STROBE Statement—checklist of items that should be included in reports of observational studies1 (© STROBE Initiative). International Journal of Public Health 53, 3-4 (2008).
40. Zhu, W., et al. Gut Microbial Metabolite TMAO Enhances Platelet Hyperreactivity and Thrombosis Risk. Cell 165, 111-124 (2016).
41. Nemet, I., et al. A Cardiovascular Disease-Linked Gut Microbial Metabolite Acts via Adrenergic Receptors. Cell 180, 862-877.e822 (2020).
42. Gupta, N., Li, W. & McIntyre, T. M. Deubiquitinases Modulate Platelet Proteome Ubiquitination, Aggregation, and Thrombosis. Arterioscler Thromb Vasc Biol 35, 2657-2666 (2015).
43. Scavone, M., et al. Platelet Adhesion and Thrombus Formation in Microchannels: The Effect of Assay-Dependent Variables. Int J Mol Sci 21, 750 (2020).
44. Witkowski, M., et al. Vascular endothelial Tissue Factor contributes to trimethylamine N-oxide-enhanced arterial thrombosis. Cardiovasc Res (2021).
45. Fiehn, O., Wohlgemuth, G. & Scholz, M. Setup and Annotation of Metabolomic Experiments by Integrating Biological and Mass Spectrometric Metadata. in Data Integration in the Life Sciences (eds. Ludascher, B. & Raschid, L.) 224-239 (Springer Berlin Heidelberg, Berlin, Heidelberg, 2005).
46. Whitmire, M. L., et al. LC-MS/MS Bioanalysis Method Development, Validation, and Sample Analysis: Points to Consider When Conducting Nonclinical and Clinical Studies in Accordance with Current Regulatory Guidances. Journal of analytical and bioanalytical techniques 2011(2011).
47. Wang, Z., et al. Impact of chronic dietary red meat, white meat, or non-meat protein on trimethylamine N-oxide metabolism and renal excretion in healthy men and women. Eur Heart J 40, 583-594 (2019).
48. Li, X. S., et al. Trimethyllysine, a trimethylamine N-oxide precursor, provides near- and long-term prognostic value in patients presenting with acute coronary syndromes. Eur Heart J 40, 2700-2709 (2019).
49. Schrottmaier, W. C., Mussbacher, M., Salzmann, M. & Assinger, A. Platelet-leukocyte interplay during vascular disease. Atherosclerosis 307, 109-120 (2020).
50. Hui, H., Fuller, K. A., Erber, W. N. & Linden, M. D. Imaging flow cytometry in the assessment of leukocyte-platelet aggregates. Methods 112, 46-54 (2017).
51. Sylvetsky, A. C., et al. Consumption of Low-Calorie Sweeteners among Children and Adults in the United States. Journal of the Academy of Nutrition and Dietetics 117, 441-448.e442 (2017).
52. EU European Union. Regulation (EU) 1169/2011 of the European parliament and of the Council of 25 Oct. 2011 on the provision of food information to consumers, amending Regulations (EC) No 1924/2006 and (EC) No 1925/2006 of the European Parliament and of the Council, and repealing Commission Directive 87/250/EEC, Council Directive 90/496/EEC, Commission Directive 1999/10/EC, Directive 2000/13/EC of the European Parliament and of the Council, Commission Directives 2002/67/EC and 2008/5/EC and Commission Regulation (EC) No 608/2004 (Text with EEA relevance). Official Journal of the European Union, 2011; L304, 18-63.
53. Sylvetsky, A. C., Walter, P. J., Garraffo, H. M., Robien, K. & Rother, K. I. Widespread sucralose exposure in a randomized clinical trial in healthy young adults. Am J Clin Nutr 105, 820-823 (2017).
54. Buerge, I. J., Buser, H. R., Kahle, M., Müller, M. D. & Poiger, T. Ubiquitous occurrence of the artificial sweetener acesulfame in the aquatic environment: an ideal chemical marker of domestic wastewater in groundwater. Environ Sci Technol 43, 4381-4385 (2009).
55. de Koning, L., et al. Sweetened beverage consumption, incident coronary heart disease, and biomarkers of risk in men. Circulation 125, 1735-1741, s1731 (2012).
56. de Koning, L., Malik, V. S., Rimm, E. B., Willett, W. C. & Hu, F. B. Sugar-sweetened and artificially sweetened beverage consumption and risk of type 2 diabetes in men. The American Journal of Clinical Nutrition 93, 1321-1327 (2011).
57. Authority, E. F. S. Outcome of the public consultation on a draft protocol for assessing exposure to sweeteners as part of their safety assessment under the food additives re-evaluation programme. EFSA Supporting Publications 17, 1913E (2020).
58. Rios-Leyvraz M, Montez J. Health effects of the use of non-sugar sweeteners: a systematic review and meta-analysis. Geneva: World Health Organization; 2022.
59. Salminen, S., Salminen, E. & Marks, V. The effects of xylitol on the secretion of insulin and gastric inhibitory polypeptide in man and rats. Diabetologia 22, 480-482 (1982).
60. Islam, M. S. Effects of Xylitol as a Sugar Substitute on Diabetes-Related Parameters in Nondiabetic Rats. Journal of Medicinal Food 14, 505-511 (2011).
61. Piscitelli, C. M., Dunayer, E. K. & Aumann, M. Xylitol toxicity in dogs. Compend Contin Educ Vet 32, E1-4; quiz E4 (2010).
62. Which Products Contain Xylitol?, Vol. 2023 (Preventive Vet, 2023). https://www.preventivevet.com/xylitol-products-toxic-for-dogs
63. Hollmann, S. & Touster, O. AN ENZYMATIC PATHWAY FROM L-XYLULOSE TO D-XYLULOSEl. Journal of the American Chemical Society 78, 3544-3545 (1956).
64. Winkelhausen, E. & Kuzmanova, S. Microbial conversion of d-xylose to xylitol. Journal of Fermentation and Bioengineering 86, 1-14 (1998).
65. Ho, A., et al. Circulating glucuronic acid predicts healthspan and longevity in humans and mice. Aging (Albany NY) 11, 7694-7706 (2019).
66. Wang, X., et al. UDP-glucose accelerates SNAI1 mRNA decay and impairs lung cancer metastasis. Nature 571, 127-131 (2019).
67. Umai, D., Kayalvizhi, R., Kumar, V. & Jacob, S. Xylitol: Bioproduction and Applications-A Review. Frontiers in Sustainability 3(2022).
68. Msomi, N. Z., Erukainure, O. L. & Islam, M. S. Suitability of sugar alcohols as antidiabetic supplements: A review. J Food Drug Anal 29, 1-14 (2021).
69. WHO. Evaluations of the Joint FAO/WHO Expert Committee on Food Additives (JECFA). (1983).

### Example 2

### The Artificial Sweetener Erythritol and Cardiovascular Event Risk

Artificial sweeteners are widely-used sugar substitutes, but little is known about their long-term effects on cardiometabolic disease risks. Here, we examined the commonly-used sugar substitute erythritol and atherothrombotic disease risk. In initial untargeted metabolomics studies in patients undergoing cardiac risk assessment (n=1,157; discovery cohort, NCT00590200), circulating levels of multiple polyol sweeteners, especially erythritol, were associated with incident (3 year) risk for major adverse cardiovascular events (MACE; includes death or non-fatal myocardial infarction or stroke). Subsequent targeted metabolomics analyses in independent US (n=2,149, NCT00590200) and European (n=833, DRKS00020915) validation cohorts of stable patients undergoing elective cardiac evaluation confirmed this association (4th vs. 1st quartile adjusted HR [95% CI], 1.80 [1.18-2.77] and 2.21 [1.20-4.07], respectively). At physiological levels, erythritol enhanced platelet reactivity in vitro and thrombosis formation in vivo. Finally, in a prospective pilot intervention study (NCT04731363), erythritol ingestion in healthy volunteers (n=8) induced marked and sustained (>2 days) increases in plasma erythritol levels well above thresholds associated with heightened platelet reactivity and thrombosis potential in in vitro and in vivo studies. Our findings reveal that erythritol is both associated with incident MACE risk and fosters enhanced thrombosis.

Erythritol is a 4-carbon sugar alcohol (a polyol) that is commonly used as a sugar substitute. It is naturally present in low amounts in fruits and vegetables¹⁶, but when incorporated into processed foods, it is typically added at levels 1000-fold higher than endogenous levels (e.g. up to 60% of food weight in some creams or pastry products^{17,18}) due to lower sweetness compared to sucrose¹⁶. The daily intake of erythritol in the total US population has been estimated to reach up to 30 g per day in some subjects based on 2013-2014 National Health and Nutrition Examination Survey (NHANES) data and FDA filings¹⁸.

Upon ingestion, erythritol is poorly metabolized and mostly excreted in the urine^{19,20} Consequently, erythritol is characterized as both a ‘zero-calorie’ or ‘non-nutritive’ sweetener, and a ‘natural’ sweetener, leading to its rapidly rising popularity and predicted doubling in market share within the sweetener sector in the next 5 years²¹. Nevertheless, little is known about circulating erythritol levels and cardiometabolic risks. Early studies have implied potential benefits, including reported antioxidant potential in animal models of diabetes²², as well as improvement in endothelial function after a 4-week ingestion of an erythritol containing drink in patients with diabetes²³. However, in a small prospective study, plasma levels of erythritol among freshman college students were associated with incident (9 month) central adiposity weight gain²⁰. In another study, erythritol levels were associated with onset of type 2 diabetes²⁴. Like all polyols, separation of erythritol from its structural isomer is difficult, hindering its analysis and quantification. A detailed examination of the relationship between erythritol and both CVD and atherothrombotic complications has not yet been reported.

Here, after initial untargeted metabolomics studies suggested circulating levels of multiple polyols, especially erythritol, were associated with incident (3 year) risk for major adverse cardiovascular events (MACE=death, myocardial infarction, stroke), we quantitatively examined the relationship between plasma levels of erythritol and incident MACE in distinct US and European validation cohorts. We also examined the impact of erythritol on platelet function in humans at levels observed following ingestion of an artificially sweetened drink, and on in vivo thrombosis potential in animal models of arterial injury.

### Results

### Untargeted Metabolomics and MACE, Discovery Cohort

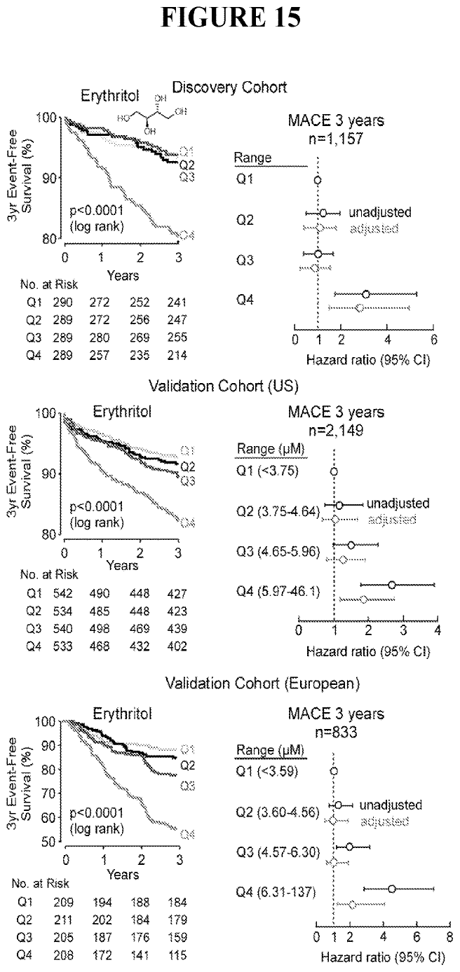

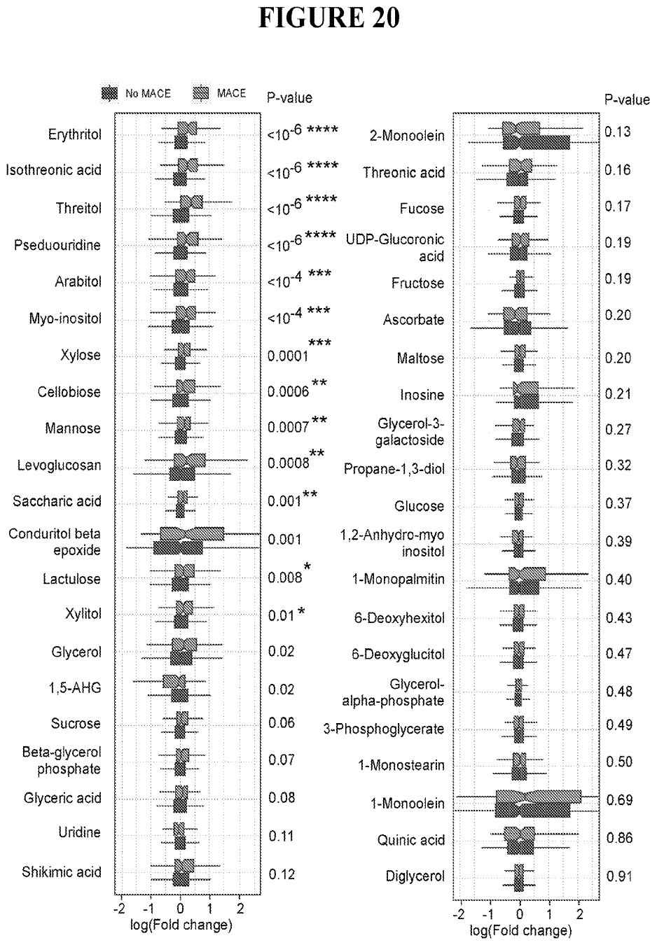

We first performed untargeted metabolomics studies in a discovery cohort (n=1,157) comprised of sequential stable patients undergoing elective diagnostic cardiac evaluation with longitudinal (3 years) outcome data (Table 4 and 5 show baseline characteristics). Among known compounds in plasma that were associated with MACE, we identified multiple polyols, including several that are commonly used as artificial sweeteners in food (FIG. 20). One of the most widely-used artificial sweeteners with rapidly increasing prevalence in processed and “keto” related foods, erythritol, was among the very top MACE-associated candidate molecules identified (HR 3.22 [95% CI 1.91-5.41], P<0.0001) (FIG. 20 and FIG. 15).

### Targeted Metabolomics Analyses of Erythritol, Validation Cohorts

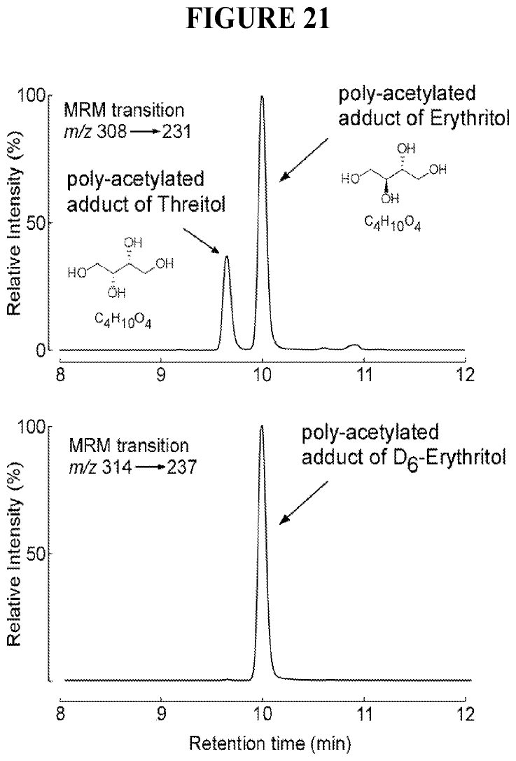

Many polyols are structural isomers of one another, sharing the same molecular weight and elemental composition, and only differing in stereochemistry, making the separation and quantification of these compounds challenging. Since untargeted metabolomics studies are only semi-quantitative and were not optimized to distinguish between structural isomers, we sought to confirm the identification of erythritol as a candidate metabolite associated with MACE risk. We thus developed a stable isotope dilution LC/MS/MS assay specifically to separate and quantify erythritol from its structural isomer (threitol) (FIG. 21). Then, we used this method to examine two independent validation cohorts comprised of subjects with longitudinal cardiovascular outcome data—namely, a US cohort (n=2,149) and a European cohort (n=833) (Table 4, 6, and 7).

TABLE 4
Clinic characteristics of the discovery and validation cohorts.

| | Discovery cohort | US cohort | European cohort |
|---|---|---|---|
| Characteristics | (n = 1157) | (n = 2149) | (n = 833) |
| Age (yr) | 65.0(55-72) | 62.9(55-71) | 75.0(66-81) |
| Male (%) | 63.6 | 64.0 | 70.1 |
| BMI (kg/m²) | 29.2(25.5-32.2) | 29.4(25.4-32.1) | NA |
| Diabetes | 22.0 | 22.1 | 27.9 |
| mellitus (%) | | | |
| Hypertension (%) | 72.2 | 70.4 | 80.4 |
| Current | 13.7 | 12.7 | 16.8 |
| smoking (%) | | | |
| Coronary artery | 75.5 | 75.0 | 69.3 |
| disease (%) | | | |
| Heart failure (%) | 16.7 | 19.4 | 17.8 |
| History of | 46.3 | 39.5 | 49.6 |
| myocardial | | | |
| infarction (%) | | | |
| Low-density | 96.0(80.0- | 96.0(77.0- | 91.5(69.0- |
| lipoprotein (mg/dl) | 115.0) | 117.0) | 122.0) |
| High-density | 34.3(28.5-41.2) | 34.3(28.2-41.7) | 48.0(39.0-60.0) |
| lipoprotein (mg/dl) | | | |
| Total cholesterol | 163.8(142.7- | 160.8(138.5- | 161.0(134.8- |
| (mg/dl) | 188.2) | 187.4) | 195.0) |
| Triglycerides | 122.0(84.0- | 114.0(84-163) | 118.0(89.0- |
| (mg/dl) | 171.0) | | 167.0) |

The baseline characteristics of participants in the discovery cohort, and both validation cohorts (US, and European) are shown. Continuous data are presented as median (25th and 75th percentiles). Categorical variables are presented as %.

TABLE 5
Clinic characteristics of the discovery cohort

| | Quartile 1 | Quartile 2 | Quartile 3 | Quartile 4 | |
|---|---|---|---|---|---|
| Characteristics | (n = 290) | (n = 289) | (n = 289) | (n = 289) | p |
| Age (yr) | 59.8(51.9-69) | 61.3(54.1-70.5) | 66.5(57.4-72.5) | 70.2(61.8-76) | <0.001 |
| Male (%) | 69.3 | 68.5 | 62.3 | 54.3 | <0.001 |
| BMI (kg/m²) | 27.8(24.9-31.6) | 28.4(25.2-31.2) | 28.7(25.6-31.9) | 28.7(25.9-33.6) | 0.04 |
| Diabetes mellitus (%) | 14.8 | 13.1 | 23.5 | 36.7 | <0.001 |
| Hypertension (%) | 59.7 | 61.9 | 67.1 | 65.4 | 0.24 |
| Current smoking (%) | 15.9 | 17.3 | 12.1 | 9.4 | 0.02 |
| CAD (%) | 75.9 | 78.2 | 70.6 | 77.5 | 0.14 |
| Heart failure (%) | 22.1 | 21.0 | 24.3 | 33.3 | 0.02 |
| Ilistory of MI (%) | 45.5 | 44.7 | 45.1 | 50.0 | 0.57 |
| LDL-c (mg/dl) | 100(82-118) | 96(80-117) | 97(83-112) | 93(76-114) | 0.05 |
| HDL-c (mg/dl) | 34(29-42) | 34(28-40) | 34(28-41) | 33.9(27-40) | 0.22 |
| Total cholesterol (mg/dl) | 163(145-187) | 160(141-188) | 164(143-185) | 161(139-188) | 0.86 |
| Triglycerides (mg/dl) | 110(75-153) | 119(82-170) | 123(85-161) | 136(96-201) | <0.001 |

The baseline characteristics of participants in the discovery cohort are shown stratified by erythritol quartiles. Continuous data are presented as median (interquartile range). Categorical variables are presented as %. BMI, body mass index; CAD, coronary artery disease; HDL, high density lipoprotein; LDL, low density lipoprotein; MI, myocardial infarction. Two-sided P values were calculated by Kruskal-Wallis test for numerical data and Chi-Square test for categorical data comparing erythritol quartiles. n=1,157.

TABLE 6
Clinical characteristics of the US validation cohort

| | Quartile 1 | Quartile 2 | Quartile 3 | Quartile 4 | |
|---|---|---|---|---|---|
| Characteristics | (n = 542) | (n = 534) | (n = 540) | (n = 533) | p |
| Age (yr) | 57.5(51-64) | 60.5(54-67) | 64.8(57-72) | 70(60-76) | <0.001 |
| Male (%) | 66.6 | 65.9 | 65.0 | 58.5 | 0.02 |
| BMI (kg/m²) | 28.5(25.1-31.9) | 28.3(25.6-31.6) | 28.7(25.9-32.5) | 28.1(25.3-32.9) | 0.30 |
| Diabetes mellitus (%) | 12.4 | 15.7 | 23.9 | 36.6 | <0.001 |
| Hypertension (%) | 61.4 | 62.7 | 64.3 | 64 | 0.76 |
| Current smoking (%) | 16.4 | 14.5 | 8.6 | 11.4 | 0.001 |
| CAD (%) | 66.7 | 71.1 | 77.4 | 85.0 | <0.001 |
| Heart failure (%) | 16.1 | 21.2 | 26.7 | 37.9 | <0.001 |
| History of MI (%) | 30.9 | 35.9 | 41.2 | 50.1 | <0.001 |
| LDL-c (mg/dl) | 99(80-120) | 99.5(79-120) | 94(76-116) | 93(73-111) | <0.001 |
| HDL-c (mg/dl) | 35(29-43) | 35(29-42) | 34(28-41) | 32(26-38) | <0.001 |
| Total cholesterol (mg/dl) | 162(140-187) | 163(143-191) | 160(137-186) | 158(132-184) | 0.005 |
| Triglycerides (mg/dl) | 103(77-139) | 111(82-161) | 119(87-159) | 131(93-188) | <0.001 |

The baseline characteristics of participants in the US validation cohort are shown stratified by erythritol quartiles. Continuous data are presented as median (interquartile range). Categorical variables are presented as %. BMI, body mass index; CAD, coronary artery disease; HDL, high density lipoprotein; LDL, low density lipoprotein; MI, myocardial infarction. Two-sided P values were calculated by Kruskal-Wallis test for numerical data and Chi-Square test for categorical data comparing erythritol quartiles. n=2,149.

TABLE 7
Clinical characteristics of the European validation cohort

| | Quartile 1 | Quartile 2 | Quartile 3 | Quartile 4 | |
|---|---|---|---|---|---|
| Characteristics | (n = 209) | (n = 211) | (n = 205) | (n = 208) | p |
| Age-yr | 67(59-76) | 72(65-79) | 77(69-82) | 79(73-85) | <0.001 |
| Male (%) | 71.8 | 73.5 | 64.9 | 70.2 | 0.25 |
| Diabetes mellitus (%) | 13.4 | 19.4 | 32.2 | 46.6 | <0.001 |
| Hypertension (%) | 75.6 | 79.6 | 82.4 | 84.1 | 0.14 |
| Current smoking (%) | 29.7 | 14.7 | 10.2 | 12.5 | <0.001 |
| CAD (%) | 64.6 | 65.4 | 68.3 | 78.8 | 0.005 |
| Heart failure (%) | 54.3 | 60.7 | 68.8 | 83.6 | <0.001 |
| History of MI (%) | 44.0 | 46.4 | 46.6 | 61.5 | 0.001 |
| LDL-c (mg/dl) | 98(71-131) | 96(71-130) | 91(72-121) | 82(64-105) | <0.001 |
| HDL-c (mg/dl) | 49(40-62) | 49(39-60) | 48(40-62) | 45(36-57) | 0.06 |
| Total cholesterol | 162(137-197) | 165(136-199) | 167(136-198) | 156(129-180) | 0.05 |
| (mg/dl) | | | | | |
| Triglycerides (mg/dl) | 121(89-172) | 111(87-163) | 115(90-160) | 133(95-174) | 0.19 |

The baseline characteristics of participants in the European validation cohort are shown stratified by erythritol quartiles. Continuous data are presented as median (interquartile range). Categorical variables are presented as %. BMI, body mass index; CAD, coronary artery disease; HDL, high density lipoprotein; LDL, low density lipoprotein; MI, myocardial infarction. Two-sided P values were calculated by Kruskal-Wallis test for numerical data and Chi-Square test for categorical data comparing erythritol quartiles. n=833.

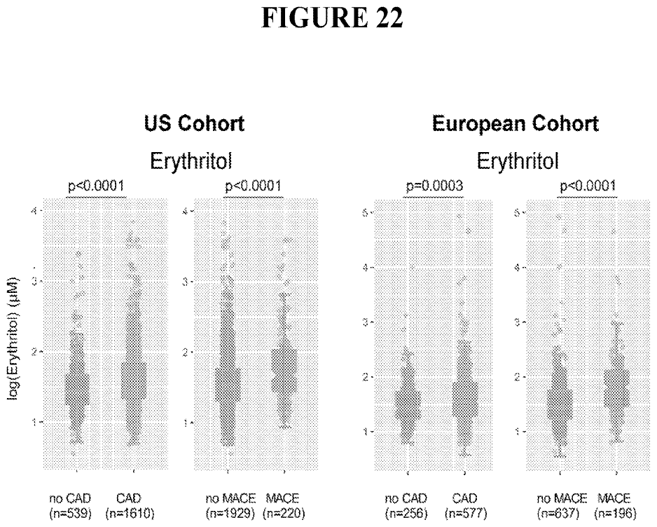

Both cohorts were enrolled at quaternary referral centers with large catchment areas with high prevalence of CVD and risk factor burden, including type 2 diabetes and obesity (i.e. individuals for whom avoidance of sweets and weight reduction efforts are routinely recommended). In both validation cohorts, plasma levels of erythritol were higher among individuals with prevalent CVD (P<0.0001 each; FIG. 22). Higher levels of erythritol were also observed among those who experienced an incident MACE over the ensuing 3 years of follow-up (P<0.0001 each; FIG. 22). Further, in both cohorts, higher incident event risk was observed with higher levels of erythritol in Kaplan-Meier analyses (FIG. 15). In Cox proportional hazard regression analyses, compared to participants in the lowest quartile of erythritol levels, those in the highest quartile had a significantly increased incident event risk in both validation cohorts (HR [95% CI]=2.64 [1.79-3.90] and 4.48 [2.86-7.02] for US cohort and European cohort, respectively, p<0.0001 each, FIG. 15). Consistent with the results observed within the discovery cohort (adjusted HR 2.95 [1.70-5.12], p<0.001, FIG. 15, Table 8), the association between erythritol levels (4^{th} quartile versus 1^{st} quartile) and incident MACE risk remained significant in both US and European validation cohorts following adjustments for cardiovascular risk factors (adjusted HR [95% CI], 1.80 [1.18-2.77] and 2.21 [1.20-4.07], P=0.007 and P=0.010, respectively) (FIG. 15, Table 9 and 10).

TABLE 8
Hazard ratios for 3-year MACE for the covariates used in
the adjusted Cox models in the discovery cohort

| | HR with 95% CI | P value |
|---|---|---|
| Age | 1.06 (0.85-1.34) | 0.59 |
| Gender | 1.10 (0.70-1.72) | 0.69 |
| Blood pressure | 1.05 (0.88-1.26) | 0.59 |
| Diabetes | 1.23 (0.79-1.91) | 0.36 |
| Smoking | 1.28 (0.73-2.25) | 0.39 |
| LDL cholesterol | 1.02 (0.83-1.26) | 0.83 |
| HDL cholesterol | 1.03 (0.80-1.31) | 0.84 |
| Triglycerides | 1.09 (0.96-1.23) | 0.18 |
| BMI | 0.90 (0.72-1.13) | 0.36 |

BMI, body mass index;
LDL, low-density lipoprotein;
HDL, high-density lipoprotein.
Two-sided P values for the adjusted Cox model are indicated.
n = 1,157.

TABLE 9
Hazard ratios for 3-year MACE for the covariates used in the
adjusted Cox models in the US validation cohort

| | HR with 95% CI | P value |
|---|---|---|
| Age | 1.60 (1.35-1.89) | <0.001 |
| Gender | 0.77 (0.57-1.05) | 0.10 |
| Blood pressure | 1.01 (0.87-1.19) | 0.86 |
| Diabetes | 0.83 (0.59-1.17) | 0.29 |
| Smoking | 2.02 (1.39-2.92) | <0.001 |
| LDL cholesterol | 1.01 (0.86-1.17) | 0.95 |
| HDL cholesterol | 0.81 (0.69-0.96) | 0.02 |
| Triglycerides | 0.88 (0.73-1.06) | 0.17 |
| BMI | 0.94 (0.81-1.11) | 0.48 |

BMI, body mass index;
LDL, low-density lipoprotein;
HDL, high-density lipoprotein.
Two-sided P values for the adjusted Cox model are indicated.
n = 2,149.

TABLE 10
Hazard ratios for 3-year MACE for the covariates used in
the adjusted Cox models in the European validation cohort

| | HR with 95% CI | P value |
|---|---|---|
| Age | 1.78 (1.40-2.28) | <0.001 |
| Gender | 1.06 (0.70-1.61) | 0.77 |
| Hypertension | 1.25 (0.74-2.11) | 0.40 |
| Diabetes | 1.53 (1.04-2.24) | 0.03 |
| Smoking | 1.66 (1 .00-2.74) | 0.05 |
| LDL cholesterol | 1.12 (0.89-1.42) | 0.32 |
| HDL cholesterol | 0.94 (0.73-1.22) | 0.66 |
| Triglycerides | 1.06 (0.87-1.29) | 0.54 |

LDL, low-density lipoprotein;
HDL, high-density lipoprotein.
Two-sided P values for the adjusted Cox model are indicated.
n = 833.

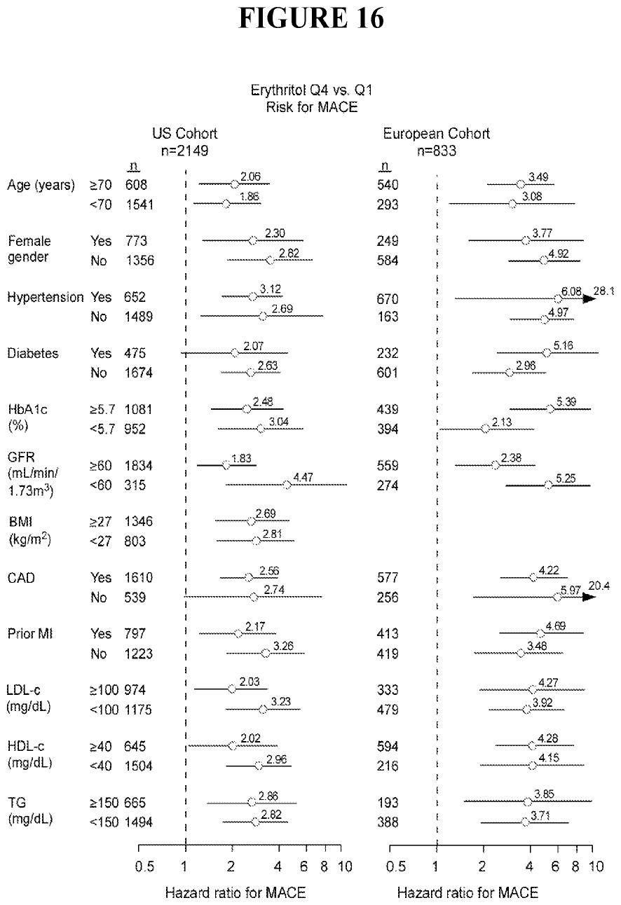

The addition of history of coronary artery disease to the model (i.e. coronary artery disease plus traditional CVD risk factors) did not materially change the association of erythritol with incident MACE (HR 1.79 [1.17-2.74] and 2.14 [1.15-3.98], P=0.007 and P=0.016 for the US and European validation cohort, respectively). Further, the association between erythritol and MACE risk was observed in both males and females alike (Table 11, 12 and 13), and was also observed to hold true among multiple different subgroups in both US and European validation cohorts (FIG. 16, Table 14 and 15). In adjusted Cox regression models where erythritol was treated as a continuous variable, erythritol was independently associated with MACE in all 3 observational cohorts (discovery, and both US and European validation cohorts, Table 16, 17 and 18). Specifically, per 1 μM increase in erythritol levels, there was a 21% and 16% increase in the adjusted HR for MACE in the US and European validation cohorts, respectively (P<0.001 and P=0.005; Table 17 and 18).

TABLE 11
Gender-stratified Cox models for 3-year MACE in the discovery cohort

| Female | Quartile 1 | | | |
|---|---|---|---|---|
| (n = 421) | (n = 106) | Q2 (n = 105) | Q3 (n = 105) | Q4 (n = 105) |
| Unadjusted | 4 | 2.35 | 1.35 (0.43-4.25) | 4.46 (1.68- |
| | | (0.83-6.65) | | |
| Adjusted HR | 1 | 2.46 | 1.46 (0.41-5.11) | 5.00 (1.68- |
| | | (0.84-7.19) | | 14.9)** |
| Male | Q1 | | | |
| (n = 736) | (n = 185) | Q2 (n = 184) | Q3 (n = 183) | Q4 (n = 184) |
| Unadjusted | 1 | 1.19 | 0.64 (0.26-1.56) | 3.25 (1.69- |
| HR | | (0.55-2.57) | | 6.24)** |
| Adjusted HR | 1 | 1.17 | 0.59 (0.24-1.46) | 2.69 (1.34- |
| | | (0.53-2.58) | | 5.41)* |

The adjustment included age, sex, diabetes mellitus, blood pressure, low-density and high-density lipoprotein cholesterol levels, triglyceride levels, current smoking status and BMI. Two-sided P values for the Cox model are indicated as follows ***P<0.001, **P<0.01. n=1,157.

TABLE 12
Gender-stratified Cox models for 3-year MACE in the
US validation cohort

| Female | Q1 | | | |
|---|---|---|---|---|
| (n = 773) | (n = 194) | Q2 (n = 194) | Q3 (n = 195) | Q4 (n = 190) |
| Unadjusted | 1 | 1.11 | 1.41 (0.73-2.74) | 2.30 (1.25- |
| HR | | (0.56-2.22) | | 4.23)** |
| Adjusted HR | 1 | 1.02 (0.52-2) | 1.18 (0.61-2.31) | 1.59 (0.79- |
| | | | | 3.2) |
| Male | Q1 | | | |
| (n = 1376) | (n = 344) | Q2 (n = 348) | Q3 (n = 340) | Q4 (n = 344) |
| Unadjusted | 1 | 1.08 | | 2.82 (1.70- |
| HR | | (0.59-1.99) | 1.7 (0.98-2.94) | 4.70)*** |
| Adjusted HR | 1 | 1.02 | 1.38 (0.79-2.43) | 1.96(1.13- |
| | | (0.55-1.90) | | 3.39)* |

The adjustment included age, sex, diabetes mellitus, blood pressure, low-density and high-density lipoprotein cholesterol levels, triglyceride levels, current smoking status and BMI. Two-sided P values for the Cox model are indicated as follows ***P<0.001, **P<0.01, *P<0.05. n=2,149.

TABLE 13
Gender-stratified Cox models for 3-year MACE in the
European validation cohort

| Female | Q1 | | | |
|---|---|---|---|---|
| (n = 249) | (n = 63) | Q2 (n = 62) | Q3 (n = 62) | Q4 (n = 62) |
| Unadjusted | 1 | 1.64 | 1.67 | 3.77 (1.61-8.80) |
| HR | | (0.64-4.2) | (0.65-4.30) | |
| Adjusted | 1 | 1.82 | 1.66 | 2.27 (0.75-6.82) |
| HR | | (0.48-7.0) | (0.53-5.19) | |
| Male | Q1 | | | |
| (n = 584) | (n = 150) | Q2 (n = 142) | Q3 (n = 146) | Q4 (n = 146) |
| Unadjusted | 1 | 1.22 | 2.09(1.17- | 4.92 (2.89- |
| HR | | (0.65-2.32) | 3.72)* | 8.35)*** |
| Adjusted | 1 | 0.98 | 0.93 (0.43- | 2.37(1.16- |
| HR | | (0.46-2.09) | 1.98) | 4.84)* |

The adjustment included age, sex, diabetes mellitus, hypertension, low-density and high-density lipoprotein cholesterol levels, triglyceride level and current smoking status. Two-sided P values for the Cox model are indicated as follows ***P<0.001, **P<0.01, *P<0.05. n=833.

TABLE 14
Hazard ratios for 3-year MACE for erythritol in different
subgroups of the US validation cohort

| | | Q4 vs. Q1 | P | P for |
|---|---|---|---|---|
| Subgroup | n | HR (95% CI) | value | interaction |
| Age ≥70 | 608 | 2.06 (1.22-3.49) | 0.007 | 0.61 |
| Age <70 | 1541 | 1.86 (1.12-3.08) | 0.02 | |
| Female | 773 | 2.30 (1.25-4.23) | 0.007 | 0.43 |
| Male | 1356 | 2.82 (1.70-4.70) | <0.001 | |
| Hypertension | 652 | 3.12 (1.25-7.76) | 0.01 | 0.95 |
| No Hypertension | 1489 | 2.69 (1.71-4.24) | <0.001 | |
| Diabetes | 475 | 2.07 (0.93-4.57) | 0.07 | 0.55 |
| No Diabetes | 1674 | 2.63 (1.69-4.09) | <0.001 | |
| HbA1C ≥5.7% | 1081 | 2.48 (1.45-4.24) | 0.001 | 0.20 |
| HbA1C <5.7% | 952 | 3.04 (1.62-5.68) | 0.001 | |
| GFR ≥60 ml/min/1.73 m³ | 1834 | 1.83 (1.18-2.85) | 0.007 | 0.11 |
| GFR <60 ml/min/1.73 m³ | 315 | 4.47 (1.82-11.0) | 0.001 | |
| BMI ≥27 kg/m² | 1346 | 2.69 (1.57-4.61) | <0.001 | 0.68 |
| BMI <27 kg/m² | 803 | 2.81 (1.58-4.98) | <0.001 | |
| CAD | 1610 | 2.56 (1.67-3.94) | <0.001 | 0.58 |
| No CAD | 539 | 2.74 (0.98-7.64) | 0.05 | |
| Prior MI | 797 | 2.17 (1.22-3.86) | 0.008 | 0.12 |
| No Prior MI | 1223 | 3.26 (1.83-5.81) | <0.001 | |
| LDL-c ≥100 mg/dL | 974 | 2.03 (1.18-3.52) | 0.011 | 0.76 |
| LDL-c <100 mg/dL | 1175 | 3.23 (1.87-5.57) | <0.001 | |
| HDL-c >40 mg/dL | 645 | 2.02 (1.04-3.92) | 0.04 | 0.75 |
| HDL-c <40 mg/dL | 1504 | 2.96 (1.82-4.80) | <0.001 | |
| Triglycerides ≥150 | 665 | 2.86 (1.38-5.92) | 0.005 | 0.41 |
| Triglycerides <150 | 1494 | 2.82 (1.76-4.53) | <0.001 | |

BMI, body mass index;
CAD, coronary artery disease;
GFR, glomerular filtration rate;
HbA1C, hemoglobin A1C;
HDL, high density lipoprotein;
LDL, low density lipoprotein;
MI, myocardial infarction.
Two-sided P values are shown for the Cox model and for interaction with the groups.
N numbers for each subset are indicated.

TABLE 15
Hazard ratios for 3-year MACE for erythritol in different
subgroups of the European validation cohort

| | | Q4 vs. Q1 HR | P | P for |
|---|---|---|---|---|
| Subgroup | n | (95% CI) | value | interaction |
| Age ≥70 | 540 | 3.49 (2.12-5.73) | <0.001 | 0.86 |
| Age <70 | 293 | 3.08 (1.22-7.78) | 0.02 | |
| Female | 249 | 3.77 (1.61-8.80) | 0.002 | 0.28 |
| Male | 584 | 4.92 (2.89-8.35) | <0.001 | |
| Hypertension | 670 | 6.08 (1.31-28.1) | <0.001 | 0.61 |
| No Hypertension | 163 | 4.97 (3.05-8.09) | 0.02 | |
| Diabetes | 232 | 5.16 (2.4-11.1) | <0.001 | 0.35 |
| No Diabetes | 601 | 2.96 (1.72-5.10) | <0.001 | |
| HbA1C ≥5.7% | 439 | 5.39 (2.96-9.81) | <0.001 | 0.05 |
| HbA1C <5.7% | 394 | 2.13 (1.06-4.25) | 0.03 | |
| GFR ≥60 ml/min/1.73 m³ | 559 | 2.38 (1.32-4.31) | 0.004 | 0.21 |
| GFR <60 ml/min/1.73 m³ | 274 | 5.25 (2.82-9.76) | <0.001 | |
| CAD | 577 | 4.22 (2.58-6.89) | <0.001 | 0.86 |
| No CAD | 256 | 5.97 (1.75-20.4) | 0.004 | |
| Prior MI | 413 | 4.69 (2.54-8.66) | <0.001 | 0.69 |
| No Prior MI | 419 | 3.48 (1.77-6.87) | <0.001 | |
| LDL-c ≥100 mg/dL | 333 | 4.27 (1.94-9.37) | <0.001 | 0.98 |
| LDL-c <100 mg/dL | 479 | 3.92 (2.25-6.83) | <0.001 | |
| HDL-c ≥40 mg/dL | 594 | 4.28 (2.4-7.62) | <0.001 | 0.99 |
| HDL-c <40 mg/dL | 216 | 4.15 (1.91-8.99) | <0.001 | |
| Triglycerides ≥150 | 193 | 3.85 (1.50-9.87) | 0.005 | 0.74 |
| Triglycerides <150 | 388 | 3.71 (1.93-7.14) | <0.001 | |

CAD, coronary artery disease;
GFR, glomerular filtration rate;
HbA1C, hemoglobin A1C;
HDL, high density lipoprotein;
LDL, low density lipoprotein;
MI, myocardial infarction.
Two-sided P values are shown for the Cox model and for interaction with the groups.
N numbers for each subset are indicated.

TABLE 16
Cox regression models in the discovery cohort

| | HR with 95% CI | P value |
|---|---|---|
| Erythritol | 1.31 (1.17-1.46) | <0.001 |
| Age | 1.20 (0.96-1.49) | 0.12 |
| Gender | 1.10 (0.70-1.73) | 0.68 |
| Blood pressure | 1.04 (0.87-1.24) | 0.67 |
| Diabetes | 1.16 (0.73-1.84) | 0.54 |
| Smoking | 1.41 (0.82-2.42) | 0.22 |
| LDL cholesterol | 1.04 (0.84-1.28) | 0.72 |
| HDL cholesterol | 1.04 (0.81-1.32) | 0.77 |
| Triglycerides | 1.11 (0.99-1.25) | 0.09 |
| BMI | 0.97 (0.78-1.21) | 0.79 |

Hazard ratio for 3-year MACE per arbitrary unit of Erythritol (untargeted metabolomics) with covariates used for adjustment.
BMI, body mass index;
HDL, high density lipoprotein;
LDL, low density lipoprotein.
Two-sided P values are shown for the adjusted Cox model.
n = 1,157.

TABLE 17
Cox regression analysis in US validation cohort

| | HR with 95% CI | P value |
|---|---|---|
| Erythritol | 1.21 (1.12-1.31) | <0.001 |
| Age | 1.69 (1.44-1.98) | <0.001 |
| Gender | 0.76 (0.56-1.03) | 0.08 |
| Blood pressure | 1.00 (0.86-1.17) | 0.98 |
| Diabetes | 0.82 (0.58-1.16) | 0.26 |
| Smoking | 1.97 (1.35-2.88) | <0.001 |
| LDL cholesterol | 1.03 (0.88-1.19) | 0.74 |
| HDL cholesterol | 0.80 (0.68-0.95) | 0.01 |
| Triglycerides | 0.89 (0.74-1.07) | 0.22 |
| BMI | 0.96 (0.82-1.12) | 0.60 |

Hazard ratio for 3-year MACE per μM of erythritol with covariates used for adjustment.
BMI, body mass index;
HDL, high density lipoprotein;
LDL, low density lipoprotein.
Two-sided P values are shown for the adjusted Cox model.
n = 2,149.

TABLE 18
Cox regression analysis in the European validation cohort

| | HR with 95% CI | P value |
|---|---|---|
| Erythritol | 1.16 (1.05-1.30) | 0.005 |
| Age | 2.01 (1.6-2.54) | <0.001 |
| Gender | 1.07 (0.7-1.63) | 0.76 |
| Hypertension | 1.25 (0.73-2.13) | 0.41 |
| Diabetes | 1.68 (1.14-2.47) | 0.009 |
| Smoking | 1.65 (1.01-2.71) | 0.05 |
| LDL cholesterol | 1.09 (0.82-1.43) | 0.57 |
| HDL cholesterol | 0.96 (0.74-1.24) | 0.75 |
| Triglycerides | 1.14 (0.95-1.36) | 0.16 |

Hazard ratio for 3-year MACE per μM of erythritol with covariates used for adjustment.
HDL, high density lipoprotein;
LDL, low density lipoprotein.
Two-sided P values are shown for the adjusted Cox model.
n = 833.

### Erythritol Exposure and Platelet Responsiveness

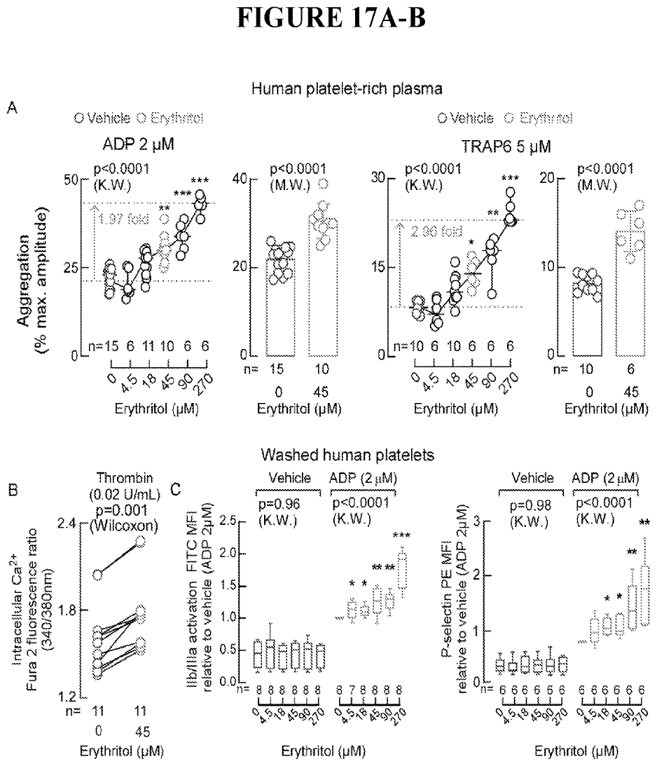

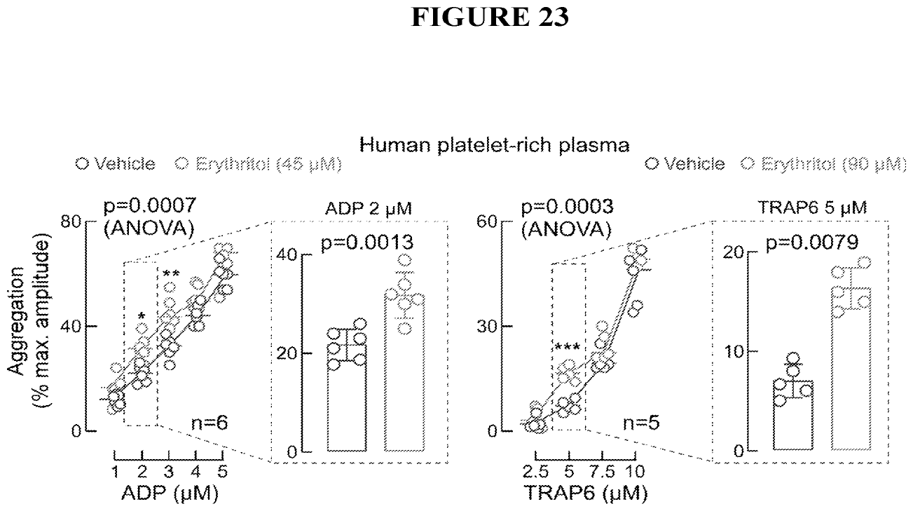

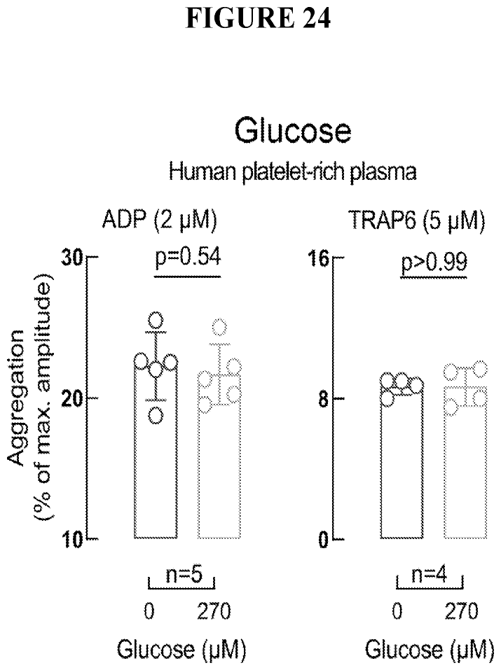

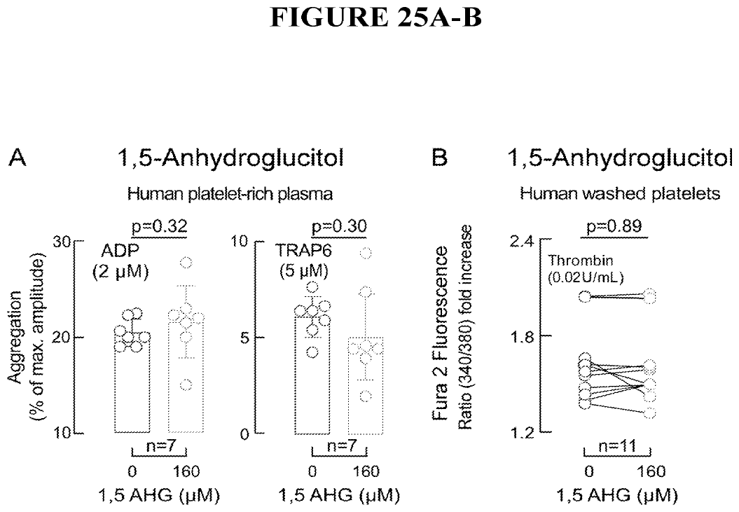

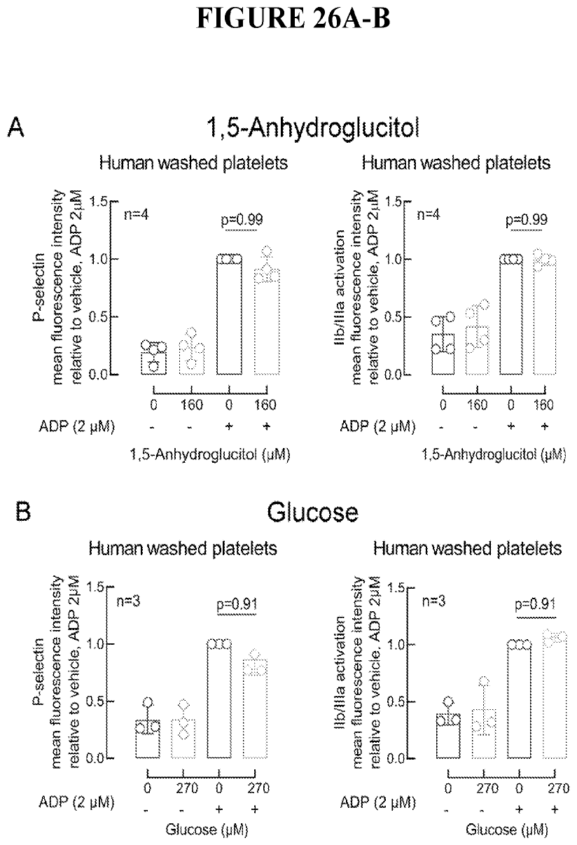

The positive association observed between circulating erythritol levels and incident thrombotic event risk led us to explore whether erythritol impacted platelet function. In these initial studies, we were careful to use erythritol concentrations within the range observed among the fasting samples examined in subjects from the US and European validation cohorts. Incubation of human platelet-rich plasma (PRP) recovered from healthy volunteers with a physiological level of erythritol versus vehicle resulted in a significant increase (i.e. leftward shift in dose response curve of different agonists) in stimulus-dependent platelet aggregation response to submaximal concentrations of two known platelet agonists: adenosine diphosphate (ADP) and thrombin receptor-activated peptide (TRAP6) (FIG. 17A and FIG. 23). In parallel experiments, a fixed submaximal dose of platelet agonist (ADP or TRAP6) was used, and the effect of increasing erythritol levels on platelet aggregometry response was monitored. Across the physiologically relevant concentration range observed in fasting plasma samples, erythritol dose-dependently enhanced platelet aggregation in PRP (FIG. 17A). In contrast, no effect on platelet aggregation responses was observed with either glucose, the most common polyol, or 1,5-anhydroglucitol (AHG), a well-established polyol surrogate of glycemic control (FIGS. 24 and 25A). Incidentally, we note that 1,5-AHG was negatively associated with cardiovascular event risks in our initial untargeted metabolomics studies (discovery cohort, FIG. 20), as well as in prior reports from large epidemiological studies²⁵. Because aggregation responses in platelet rich plasma can be influenced by factors independent of platelets, and to directly test whether erythritol impacts platelet function, we isolated platelets from healthy volunteers and then examined the effect of brief (30 min) exposure to physiological levels of erythritol versus either vehicle control or 1,5-AHG as a control on multiple indices of platelet functional responses. Notably, erythritol, but not 1,5-AHG, enhanced intracellular cytosolic Ca²⁺ concentrations in washed human platelets following exposure to submaximal (0.02 U/mL) thrombin (FIG. 17B, FIG. 25B). Similarly, exposure of washed human platelets to erythritol, but not vehicle, glucose, or 1,5-AHG, caused a dose-dependent enhancement in multiple platelet activation phenotypes, including ADP-stimulated P-selectin surface expression and glycoprotein α2β3 (GP IIb/IIIa) activation (FIGS. 17B and 17C, FIG. 26).

### Erythritol and Thrombosis Potential in Human Whole Blood, and In Vivo

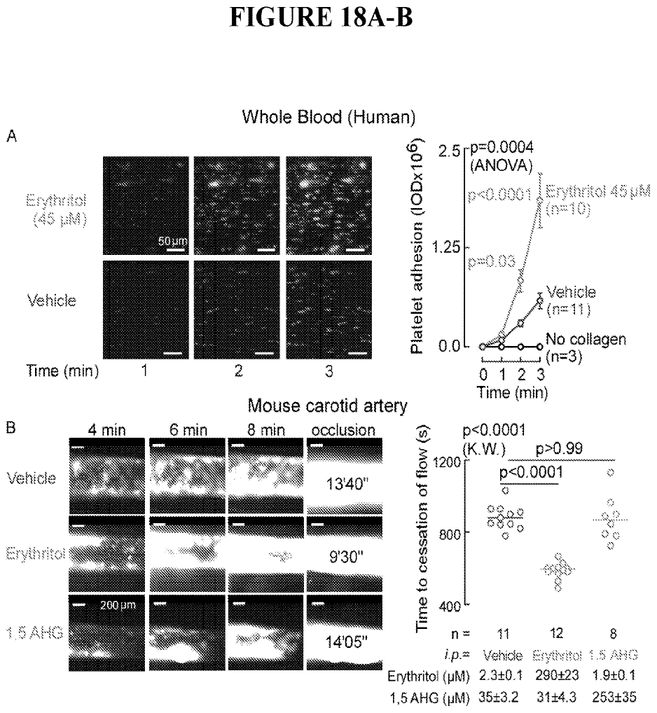

We further examined the effect of erythritol on platelet adhesion, the initial step in clot formation, in human whole blood under physiological shear conditions using a microfluidics device. Erythritol elicited significant enhancement in the rate of collagen-dependent platelet adhesion and thrombus formation (FIG. 18A). The impact of erythritol on in vivo thrombosis potential was further examined in mice by monitoring both the rate of clot formation and the time to cessation of blood flow using a FeCl₃-induced carotid artery injury model²⁶. Notably, when compared to either saline (vehicle) or 1,5-AHG, elevation of circulating erythritol levels elicited marked enhancement in the rate of thrombus formation, as well as significant reduction in the time to cessation of blood flow following arterial injury (FIG. 4B).

### Postprandial Levels of Erythritol in Healthy Individuals

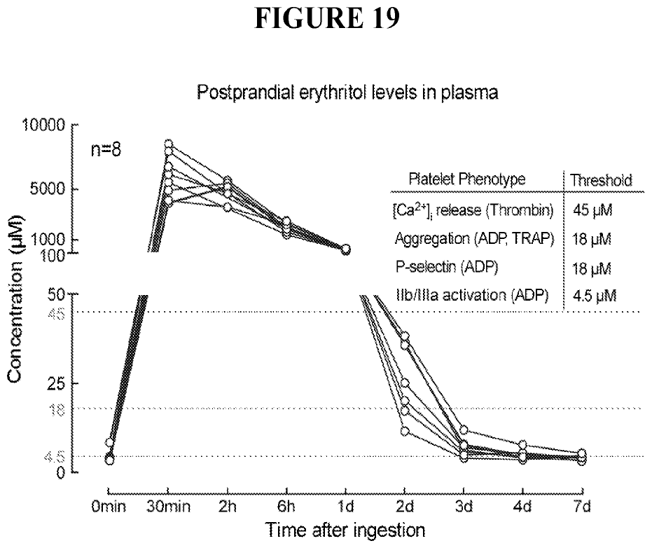

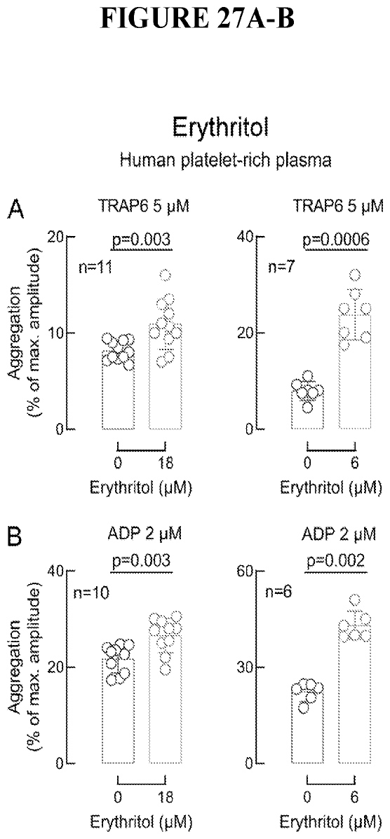

Since numerous “zero calorie” or “keto” friendly prepared foods and beverages can possess relatively large quantities of erythritoli^{17,18}, we thought it would be of interest to assess the physiological range in circulating erythritol levels observed following a relevant dietary exposure. We thus examined postprandial erythritol plasma levels in healthy participants (n=8) following an erythritol-sweetened drink (30 g), an erythritol exposure comparable to a single can of commercially available artificially sweetened beverage, a pint of keto ice cream, or other foods or beverages containing erythritol. While plasma levels of erythritol were low at baseline (median [25th and 75th percentiles], 3.84 [3.27-4.14]μM), they remained 1000-fold higher (millimolar levels) for hours after ingestion (e.g. at 30 min, 5.85 [4.30-7.68] mM), and remained significantly elevated for over 2 days in all participants examined (FIG. 19). Notably, the elevation in erythritol levels observed remained well above thresholds observed for concentrations of erythritol that elicit significant increases in multiple indices of platelet function, including stimulus-dependent (thrombin) increases in intracellular calcium (45 μM, FIG. 17), ADP- or thrombin-stimulated aggregometry responses (18 μM each, FIG. 27), and stimulus-dependent enhancement in P-selectin or activated GP IIbIIIa surface expression (18 μM and 4.5 μM, respectively, FIG. 17).

In the present Example, we used an initial untargeted metabolomics approach as a discovery platform to identify circulating metabolites associated with incident CVD event risk. While untargeted metabolomics is only semi-quantitative in nature, these qualitative results suggested that multiple polyols in general, and erythritol specifically, are associated with incident CVD risks. Across both US and European validation cohorts, we confirmed that circulating levels of erythritol were associated with incident adverse cardiovascular event risk independent of traditional CVD risk factors. Sensitivity analyses showed that this association remained significant in multiple different subgroups across both cohorts. Furthermore, through mechanistic studies, multiple lines of evidence indicate that elevated erythritol levels can directly contribute to heightened platelet reactivity and thrombosis risk by enhancing platelet intracellular calcium release and aggregation in response to multiple agonists. Specifically, the use of a preclinical in vivo thrombosis model similarly indicates higher rates of clot formation and increased thrombosis potential following arterial injury when plasma erythritol levels are elevated.

Erythritol is endogenously produced by the pentose phosphate pathway^{20,27}, and the metabolite is readily observed in circulation. We speculate that erythritol levels in both validation cohorts originate from a combination of ingestion and endogenous production. While fasting samples in the US validation cohort (where enrollment largely preceded proliferation of erythritol in processed foods) likely reflect endogenous levels, our intervention study clearly shows prolonged elevation of erythritol after ingestion. So even in fasting individuals, erythritol levels may reflect post-prandial levels (e.g. in the more recently recruited EU validation cohort that enrolled participants well into 2018).

Since the discovery of microbial fermentation processes that allowed for large-scale industrial production of erythritol in the 1990s, the sweetener has increasingly been added to processed foods, with rapid approval for its use in many countries around the world (and applications are still increasing)²⁸. Potential benefits of eythritol's use as an artificial sweetener that contribute to its rapidly growing market penetration include a high digestive tolerance (a daily dose up to 1 g/kg is well tolerated), presumed non-carcinogenic and antioxidant effects, and perceived qualitative sweetness improvements that make erythritol commercially used to sweeten food both alone and as a bulk sweetener in combination with other high intensity sweeteners^{16,22,29,30}. Studies report erythritol has no short-term insulinemic or glycemic effects, therefore it has been considered well-suited for patients with impaired glucose control or obesity³⁰. Erythritol's safety has been assessed by short-term animal toxicity studies and reported human clinical studies with ingestion up to 4 weeks^{23,31}. Based on these studies, along with its natural occurrence both endogenously in human tissues and in food (albeit at levels 1000-fold lower than used as additive to processed foods), erythritol is “generally recognized as safe” by both the EU and the FDA^{17,18}. The World Health Organization (WHO)/Food and Agriculture Organization of the United Nations (FAO) Expert Committee on Food Additives assigned an acceptable daily intake that is “not specified”¹⁷. The FDA does not require disclosure of erythritol content in food products, making its levels in foods as an additive hard to track. Many observational epidemiological studies report that artificial sweetener use is associated with various adverse health outcomes including CVD mortality^{9-11,13,14,32-35}, while others do not^{36,37}. One possible explanation for these conflicting findings is the difficulty in reliably quantifying dietary artificial sweetener consumption. In addition, “artificial sweeteners” are often (typically) reported in aggregate due to non-disclosure policies on food-labels. This limits the specification of individual sweeteners on labels, and also the ability to monitor adverse long-term outcomes with individual sweeteners in clinical studies. Further, this has led to difficulties in linking the amount of dietary artificial sweetener use with circulating levels. The present results highlight the need to establish reporting requirements, safety profiles, and margins of daily intake amounts given that broad consumption continues to increase. Public policy decisions need to be evidence-based and better informed.

In one randomized intervention study, the artificial sweeteners saccharin and sucralose were linked to impaired glycemic responses in participants³⁸. In a population based prospective cohort study with repeated dietary records, ingestion of multiple artificial sweeteners (e.g. aspartame, acesulfame potassium, and sucralose) was associated with cardiovascular disease risk³⁹. Meta analyses of both the limited number of brief randomized controlled trials (median follow-up of only 3 to 6 month) and observational studies with artificial sweeteners concluded that low calorie sweeteners and non-nutritive sweeteners do not provide the intended benefits, and instead are associated with adverse cardiometabolic phenotypes, including weight gain, increased body fat, type 2 diabetes, and cardiovascular events^{40,41}, while other clinical trials meta analyzed suggested potential small improvements^{42,43}. Meanwhile, intervention safety studies on artificial sweeteners are conducted over relatively short durations, and have been criticized both for inadequately capturing long-term exposure, and for differing from real-life practice⁴⁴.

A previous study employing untargeted metabolomics specifically reported relative erythritol levels. Using samples from the Atherosclerosis Risk in Communities (ARIC) study, it was proposed that 19 different analytes (one of which was erythritol), when cumulatively included in a risk score, could provide additive predictive value for incident coronary heart disease⁴⁵. The general lack of reporting of erythritol in the literature might be due in part to difficulties in quantification of erythritol, like other polyols, due to its structural isomers. The present studies suggest that following ingestion of an artificially sweetened food harboring typical levels of erythritol as artificial sweetener, plasma levels of erythritol remain elevated for many days, well above the thresholds necessary to enhance stimulus-dependent platelet reactivity, even amongst healthy volunteers. Our erythritol pharmacokinetics studies served to identify postprandial peak levels and the time course of elimination. Based on these studies in a limited number of people, all subjects included had elevated plasma levels for approximately 2 days (FIG. 19).

In summary, the present studies suggest that trials investigating the impact of crythritol specifically, and artificial sweeteners in general, with appropriate duration of follow-up for clinically relevant outcomes, are needed. Following exposure to dietary erythritol, a prolonged period of potentially heightened thrombotic risk may occur. This is of concern given that the very subjects for whom artificial sweeteners are marketed (patients with diabetes, obesity, history of CVD and impaired kidney function) are those typically at higher risk for future CVD events.

### Methods

### Ethics Approvals

Four distinct clinical studies were performed. All human subjects gave written informed consent, and all human studies performed abided by the Declaration of Helsinki. The Institutional Review Board of the Cleveland Clinic, or the ethics committee of Charité-Universitatsmedizin Berlin approved all study protocols (GeneBank IRB 4265, European validation cohort EA1/135/16, COSETTE IRB 21-005, healthy volunteer blood donors for platelet related studies IRB 09-506). All animal model studies were approved by the Institutional Animal Care and Use Committee at the Cleveland Clinic.

### Study Design

### Discovery Cohort

In the first clinical study, untargeted metabolomics analyses were performed on plasma samples from a discovery cohort including stable subjects undergoing elective cardiac catheterization (n=1,157) to identify circulating analytes whose levels in semi-quantitative analyses were associated with incident cardiovascular disease (CVD)-related risks. The discovery cohort consisted of sequential stable adult patients undergoing cardiac risk assessment for symptom evaluation at a quaternary referral center (Cleveland Clinic) between 2001-2007. Participants were monitored for MACE (Major Adverse Cardiovascular Event) outcomes adjudicated up to 3 years (GeneBank at the Cleveland Clinic; clinicaltrials.gov identifier: NCT00590200), as previously described^{46,47}.

### US Validation Cohort

In the second clinical study, stable isotope dilution liquid chromatography tandem mass spectrometry (LC/MS/MS) was used to quantify erythritol in serum samples from a non-overlapping cohort of independent subjects (US Cohort, n=2,149) from GeneBank at the Cleveland Clinic (clinicaltrials.gov identifier: NCT0059020)^{46,47}. The subjects enrolled in GeneBank have broad geographic catchment from over 40 states throughout the US. All participants had extensive clinical and longitudinal outcome data collected, including adjudicated adverse cardiovascular events over the ensuing 3 years after enrollment. MACE (Major Adverse Cardiovascular Event) was defined as death, non-fatal myocardial infarction, or nonfatal cerebrovascular accident (stroke) following enrollment. Coronary artery disease (CAD) was defined as any clinical history of myocardial infarction, coronary revascularization (including percutaneous coronary intervention, coronary artery bypass surgery), or angiographic evidence of significant stenosis (≥50%) in 1 or more major coronary arteries. Estimated glomerular filtration rate (eGFR) was calculated via CKD-EPI equation⁴⁸, and in sensitivity analyses, examined above versus below the cut point for chronic kidney disease stage III (<60 mL/min/1.73 m²).

### European Validation Cohort

In a third clinical study (the European Cohort, n=833), serum erythritol levels were quantified by stable isotope dilution LC/MS/MS in samples from sequential patients undergoing elective diagnostic coronary angiography due to (suspected) chronic coronary syndromes enrolled in the observational LipidCardio study between 2016-2018 at the Charité University Hospital, Campus Benjamin Franklin (registered under German Clinical Trial Register (drks.de); identifier: DRKS00020915) with a follow-up for 3 years⁴⁹. As a quaternary referral center, Charité University Hospital is centrally located in Europe, and subjects enrolled in the LipidCardio study have large geographic catchment, with residences throughout Europe. eGFR was calculated via CKD-MDRD equation⁴⁸. For the European validation cohort, there was a total of 833 samples available with MACE outcome data. All samples were used for the erythritol LC/MS/MS measurement.

### Erythritol Intervention Study

In a fourth study, the first phase of the Erythritol Intervention Study (COSETTE, clinicaltrials.gov number: NCT04731363), prospectively recruited healthy volunteers ingested a standard-size artificially sweetened beverage (300 mL) containing 30 g of erythritol with instructions to consume the drink within 2 minutes, and blood samples were collected at scheduled post-prandial time points for crythritol measurement. For each participant in the erythritol intervention study (n=8), baseline blood samples were first obtained after overnight (≥8 hours) fasting. Information provided during the FDA approval processes suggests a daily erythritol consumption of up to >30 g per day in some of the US population based on National Health and Nutrition Examination Survey data¹⁸. Therefore, participants were given 30 g erythritol dissolved in 300 mL water. Serial venous blood sampling was performed up to 7d after baseline. The reported pharmacokinetics data in this manuscript (FIG. 19) were acquired as the first part of COSETTE to identify both the timing of peak plasma levels of erythritol after ingestion, and the time course of erythritol elimination. The registration at Clinicaltrials.gov and the IRB protocol for COSETTE also include a distinct non-overlapping second separate study that assesses platelet functional changes after ingestion.

### Platelet Related Studies

For in vitro studies using human platelet-rich plasma, washed platelets or whole blood, healthy adults (n=55 total) with no chronic illness were consented for blood donation using a distinct IRB protocol.

### Aggregometry Assay in Platelet-Rich Plasma

Platelet rich plasma (PRP) and platelet poor plasma (PPP) was prepared as previously described with sodium citrate (0.109 M) as anticoagulant⁵¹. Platelets were counted and concentrations adjusted to 2×10⁸/mL with PPP. PRPs were pre-incubated with erythritol (Catalogue #E7500, Sigma, St. Louis, MO, USA), glucose (Catalogue #G7021, Sigma, St. Louis, MO, USA) or 1,5-AHG (Catalogue #29874, Cayman Chemical, Ann Arbor, Michigan, USA) (at indicated concentrations) or vehicle (saline) for 30 min at 22° C. After pre-incubation PRPs were maintained in suspension with constant stirring (600 rpm) at 37° C. and platelet aggregation was initiated using ADP (up to 5 μM, Catalogue #384, Chronolog, Havertown, PA, US) or TRAP6 (TFLLR-NH2, up to 10 M, Catalogue #464, Tocris, Bristol, UK).

### Intracellular Calcium Measurements

Washed platelets for intracellular Ca²⁺ measurements were prepared by adding 100 nM prostaglandin E1 (PGE-1, Catalogue #P5512, Sigma, St. Louis, MO, USA) to PRP and centrifugation at 500×g, 20 min at 22° C. as previously described^{26,51}. The platelet pellet was washed with a modified phosphate buffer saline (NaCl (137 mM), KCl (2.7 mM), Na2HPO4 (12 mM), MgCl2 (1 mM), and glucose (5.5 mM), pH 7.4) with PGE-1 (100 nM), and spun again at 500×g for 20 min. The platelets were re-suspended in modified Hank's buffered salt solution (HBSS-BSA-glucose; NaCl (0.137 M), KCl (5.4 mM), Na2HPO4 (0.25 mM KH2PO4 (0.44 mM), CaCl₂) (1.3 mM), MgSO4 (1.0 mM), NaHCO₃ (4.2 mM), glucose (5 mM) and BSA (0.1%)) with 100 nM PGE-1 and incubated with Fura 2-AM (1 mM) at 22° C. for 30 min. Excess Fura 2-AM was removed by additional centrifugation at 500×g for 30 min. The platelets were then re-suspended in modified Hank's buffered salt solution and incubated with erythritol or 1,5-AHG at the indicated concentrations or vehicle for 30 min at 22° C. Intra-cellular calcium release was induced by submaximal concentration of thrombin (0.02 U/mL) and changes monitored via Fura 2-AM fluorescence using 340/380 nm dual-wavelength excitation and an emission of 510 nm.

### Platelet Flow Cytometry Assay

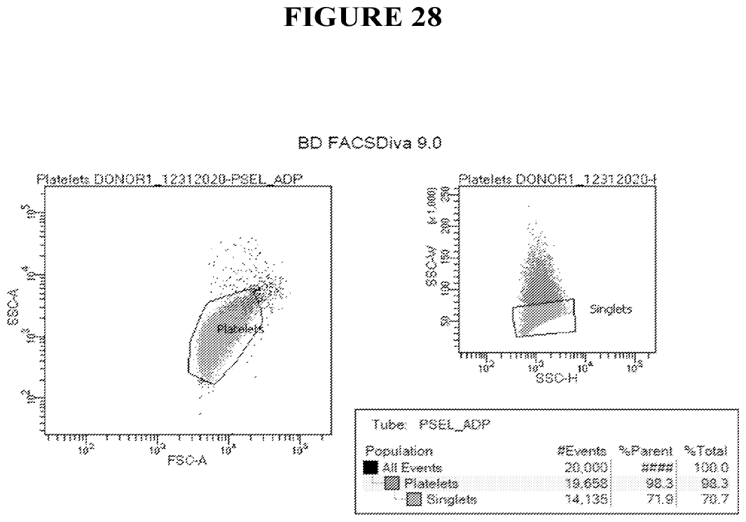

Washed platelets and antibody staining for flow cytometry was performed as described previously using sodium citrate (0.109 M) as anti-coagulant⁵¹. Washed platelets were separated by centrifugation at 500×g for 10 min and re-suspended in modified Hank's buffered salt solution without PGE1. Final platelet suspensions (100 μL; 2×10⁸ platelets/mL) were then pre-incubated with erythritol, glucose or 1,5-AHG (at indicated concentrations) for 30 min at 22° C. Platelets were then stimulated with 2 μM ADP for 10 min and incubated with PE conjugated anti-P-selectin (CD62P-PE, Catalogue #555524, BD PharMingen, San Diego, CA, USA; 2.5 μL/100 μL) or Fluorescein isothiocyanate (FITC) conjugated PAC1 (binds only to active conformation of GP IIb/IIIa, Catalogue #3 40507, BD PharMingen, San Diego, CA, USA; 5 μL/100 μL) or isotype control antibody (PE IgG isotype control, Catalogue #555749 or FITC IgM Isotype control, Catalogue #555583, BD PharMingen, San Diego, CA, USA) in the dark for 20 min. The platelet suspensions were then fixed with 100 μL of 2% paraformaldehyde. Data was acquired on a flow cytometer (FACS LSR Fortessa, BD Biosciences, Franklin Lakes, New Jersey, USA). Twenty thousand (20,000) events were collected. The data was analyzed with FACSDiva Software (v.9.0) (BD Biosciences). Platelets were gated to exclude doublets (FIG. 28) and the raw mean fluorescent intensity (MFI) of either P-selectin (CD62P) or PAC-1 was quantified.

### Whole Blood In Vitro Thrombosis Assay

Microfluidic shear flow experiments were performed using the Cellix Microfluidics System (Cellix, Dublin, Ireland) as previously described⁵-53. Where indicated, each micro channel of a Vena8 Fluoro+ biochip was coated with type 1 collagen (15 μL; 50 μg/mL). Images were collected using an HC Plan Apo 20X/0.7NA lens on a Leica DMI6000 inverted microscope equipped with an environmental chamber and a Hamamatsu ImagEM cooled CCD camera. Whole blood was incubated with an Alexa Fluor®488-conjugated anti-human CD42b antibody (catalogue #303914, Biolegend, San Diego, CA, USA; 5 μL/100 μL blood) and was pretreated with erythritol (45 μM) or normal saline (control) for 30 min at 22° C. Blood was then perfused over chips coated with or without immobilized type 1 collagen at a physiological shear rate (60 dynes/cm²) using a multi-channel microfluidic device for 3 min. Images of fluorescent platelets adhering to the collagen coating were captured every 5 s during that time. At the end of the experiment, the tube containing the whole blood was removed and the 1×PBS in the biochip reservoir was drawn through the channel at 20 dynes/cm². Five images were captured along the length of the channel during that time. Platelet activation and adherence to the collagen surface was then quantified with computer assisted tomographic analyses. Briefly, images of CD42b stained thrombi were quantified using Image Pro plus software v7.0.0 (Media Cybernetics, Rockville, Maryland, USA). Intensity threshold was chosen to select for specific staining and quantified for integrated optical density (IOD, Area×Intensity).

### Murine Model for Carotid Artery FeCl3 Injury Thrombosis Assay

The common carotid artery FeCl₃ induced injury model was performed as previously described⁵⁴. 12-14 weeks old BL/6J mice were injected with vehicle (normal saline), erythritol (25 mg/kg) or 1,5-AHG (25 mg/kg) and anesthetized with 100 mg/kg ketamine+10 mg/kg xylazine. Rhodamine 6G (100 μL; 0.5 mg/mL, catalogue #252433, Sigma, St. Louis, MO, USA) was injected into the right jugular vein to label platelets. The left carotid artery was then injured with a Whatman filter paper of 1 mm² size containing 10% FeCl₃ (Catalogue #157740, Sigma, St. Louis, MO, USA) for 1 min. Intravital fluorescence microscopy equipped with video recording was used to monitor thrombus formation in real time. Time to cessation of blood flow through clot formation for all studies was determined by visual inspection of captured video by two independent investigators in real time. Data for the in vivo thrombosis were collected by Streampix 7—Multiple Camera DVR Software (NorPix Inc, Montreal, Canada). Animals were immediately euthanized after data acquisition.

### Untargeted GC-MS Analysis of Human Plasma Samples

### Sample Preparation

1 mL of chilled (−20° C.) extraction solution (acetonitrile/isopropanol/water, 3:3:2, v/v/v) was added to 30 μL plasma aliquots. After vortexing for 10 s and shaking for 5 min at 4° C., the samples were centrifuged for 2 min at 14,000 ref. Three aliquots (each 300 μL) were taken: one for GC-MS analysis and two for backup samples. GC-MS aliquots were evaporated to dryness followed by re-suspending with 450 μL 50% acetonitrile. After centrifugation for 2 min at 14,000 ref the supernatants were pipetted to new Eppendorf tubes followed by evaporation to dryness. A two-step derivatization was used prior to GC-MS analysis. Methoxyamine hydrochloride in pyridine (10 μL; 40 mg/mL) was added to dried samples and shaken for 1.5 hours at 30° C. Next, 60 μL of N-Methyl-N-(trimethylsilyl)trifluoroacetamide (MSTFA) with fatty acid methyl ester (FAME) mixture was added to each sample and shaken for 0.5 hour at 37° C. After centrifugation (2 min at 14,000 ref), the content was submitted to GC-MS analysis.

### GC-MS Analysis

The system was composed of an MPS2 automatic liner exchange system (Gerstel, Mulheim an der Ruhr, Germany), an Agilent 7890A GC system, and a time-of-flight Pegasus III mass spectrometer (Leco, St. Joseph, MI, USA). Injection parameters were as follows: injection volume, 0.5 μL; injector temperature, 50° C. ramped to 275° C. at a rate of 12° C./s; helium carrier gas flow, 1 mL/min; splitless period, 25 s. For GC separation a 30 m×0.25 mm, 0.25 μm Rtx5Sil MS (Restek, Bellefonte, PA, USA) capillary column including an additional 10 m integrated guard column (Restek) was used with an oven temperature program: 50° C. (1 min), 20° C./min to 330° C. (5 min). MS parameters were as follows: electron ionization, −70 eV; acquisition rate, 17 spectra/s; mass range, m/z 85-500; MS ion source temperature, 250° C.; transfer line temperature, 280° C. For data acquisition, ChromaTOF 2.32 (Leco) software was used.

### Data Processing

Raw data files were processed using the metabolomics BinBase database⁵⁵. All database entries in BinBase were matched against the UC Davis metabolomics center's mass spectral library.

### Targeted Analysis of Selected Polyols in Plasma

Stable-isotope-dilution LC/MS/MS was used for quantification of erythritol, threitol, and 1,5-AHG in human and mouse plasma. Ice cold methanol (800 μL) and internal standards (D₆-erythitol and ¹³C₆-1,5-AHG) were added to the plasma samples (20 μL), followed by vortexing and centrifuging (21,000×g; 4° C. for 15 min). The clear supernatant (800 μL) was transferred into a clean, labeled glass tubes (Borosilicate glass 12×75 mm) and dried in a speed vacuum concentrator (Speed vac plus, SC210, Thermo Sevant). The dry residue was reconstituted in acetic anhydride (100 μL) and 4-Dimethylaminopyridine (DMAP) in pyridine (100 μL; 1 mg/mL), sealed with safety caps, vortexed and heated (45 min at 80° C.) followed with drying under nitrogen. Dried residues were dissolved in HCl in water (0.1 M; 0.5 mL) and extracted with ethyl acetate (2 mL). Ethyl acetate layer was transferred into a clean glass tubes (Borosilicate glass 12×75 mm) and dried under nitrogen. The dry residue was reconstituted in ammonium formate in a mixture of methanol:water (100 μL; 50:50 v/v with 10 mM ammonium formate), tubes were vortexed and liquid was transferred to glass vials with micro-insets and caped. LC/MS/MS analysis was performed on a chromatographic system consisting of two Shimadzu LC-30 AD pumps (Nexera X2), a CTO 20AC oven operating at 30° C., and a SIL-30 AC-MP autosampler in tandem with a triple quadruple mass spectrometer (8050 series, Shimadzu Scientific Instruments, Inc., Columbia, MD, USA). For chromatographic separation, a Kinetex C18 column (50 mm×2.1 mm; 2.6 m) (Cat #00B-4462-AN, Phenomenex, Torrance, CA) was used. Solvent A (10 mM ammonium formate and 0.1% formic acid in water) and B (10 mM ammonium formate and 0.1% formic acid in acetonitrile:water 95:5) were run using the following gradient: 0.0 min (0% B); 0.0-11.0 min (25% B); 11.0-14.0 min (25% B→30% B); 14.0-17.0 min (30% B→35% B); 17.0-19.0 min (35% B); 19.0-22.0 min (35% B→40% B); 22.0-22.5 min (100%); 22.5-25 min (100% B); 25.0-26.0 min (100% B→0% B); 26.0-28.0 min (0% B) with flow rate of 0.35 mL/min and an injection volume of 1 μL. Electrospray ionization in the positive mode was used with multiple reaction monitoring (MRM) for detection of endogenous and stable isotope labeled internal standards. The following transitions were used: m/z 308.0 [M+4xC₂OH₂+NH₄]⁺→231.0 for threitol and erythritol, m/z 314.0 [M+4xC₂OH₂+NH₄]⁺→237.0 for D₆-erythritol; m/z 360.1 [M+4xC₂OH₂+NH₄]⁺→273.3 for 1,5AHG; m/z 356.1 [M+4xC₂OH₂+NH₄]⁺→279.3 for ¹³C₆-1,5-AHG; The following ion source parameters were applied: nebulizing gas flow, 3 l/min; heating gas flow, 10 L/min; interface temperature, 300° C.; desolvation line temperature, 250° C.; heat block temperature, 400° C.; and drying gas flow, 10 L/min. Limit of detection (LOD) and limit of quantification (LOQ) were as follow: threitol:0.048 and 0.160 μM; erythritol: 0.026 and 0.089 μM; 1,5-AHG: 0.011 and 0.035 M; respectively. Three quality control samples were run with each batch of samples and inter-batch variations expressed as coefficient of variation (CV) were less than 7% for all analytes monitored. Data were collected and analyzed by LabSolution 5.91 software (Shimadzu).

### Statistical Analysis

Continuous variables were summarized as median (25th and 75th percentiles), and categorical variables are presented as %. For group comparisons of patient characteristics, Kruskal-Wallis test was performed for numerical data and Chi-Square test for categorical data. Given the relatively small sample sizes, we did not feel that the assumptions behind parametric approaches we considered (particularly, normality of models' error terms) were sufficiently well justified and so non-parametric models were preferred for most in vitro and in vivo studies. Mann-Whitney U-test or Wilcoxon matched-pairs signed rank test were applied to continuous variables. Kruskal-Wallis test with Dunn's post hoc test was used for multiple comparisons. Kaplan-Meier analysis with Cox proportional-hazards regression was used for time-to-event analysis to determine hazard ratios (HR) and 95% confidence intervals (CI) for MACE. Adjustments included traditional cardiovascular risk factors that are known to predict CVD event risk^{56,57}: age, sex, diabetes mellitus, systolic blood pressure (in the European Cohort hypertension), low-density and high-density lipoprotein cholesterol levels, triglyceride levels, and current smoking status. For the discovery cohort and US validation cohort, the adjustment also included body mass index (BMI), in addition to the aforementioned variables. The R built-in cox.zph( ) function was used to check for proportionality assumptions in Cox models, by using the Schoenfeld residuals against the transformed time. There was no evidence against proportionality. Two-way ANOVA with Sidiák's multiple comparison post hoc test was used for multiple-group comparisons of aggregometry data using different concentrations of agonists. For analysis of collagen-dependent platelet adhesion in whole blood, we performed a two-way repeated measures ANOVA with Sidák's multiple comparison post hoc test. False discovery rate corrected P values for metabolite levels in the discovery cohort were calculated using the Benjamini-Hochberg method. All reported measurements were taken from distinct samples (for whole blood in vitro thrombosis experiments (FIG. 19A) individual biological replicates were followed over 3 min). Data analyses were performed with R software (version 4.0.2) and GraphPad Prism software (version 9.0). All reported P values are two-sided. A two-sided P<0.05 was considered statistically significant.

### References for Example 2

1. Abarca-Gómez, L., et al. Worldwide trends in body-mass index, underweight, overweight, and obesity from 1975 to 2016: a pooled analysis of 2416 population-based measurement studies in 128-9 million children, adolescents, and adults. The Lancet 390, 2627-2642 (2017).
2. Sylvetsky, A. C. & Rother, K. I. Trends in the consumption of low-calorie sweeteners. Physiol Behav 164, 446-450 (2016).
3. Buerge, I. J., Buser, H. R., Kahle, M., Müller, M. D. & Poiger, T. Ubiquitous occurrence of the artificial sweetener acesulfame in the aquatic environment: an ideal chemical marker of domestic wastewater in groundwater. Environ Sci Technol 43, 4381-4385 (2009).
4. Roberts, A. The safety and regulatory process for low calorie sweeteners in the United States. Physiol Behav 164, 439-444 (2016).
5. Mortensen, A. Sweeteners permitted in the European Union: safety aspects. Scandinavian Journal of Food and Nutrition 50, 104-116 (2006).
6. Gardner, C., et al. Nonnutritive sweeteners: current use and health perspectives: a scientific statement from the American Heart Association and the American Diabetes Association. Circulation 126, 509-519 (2012).
7. British Dietetic Association. Policy statement—The Use of Artificial Sweeteners. Available at: https://www.bda.uk.com/uploads/assets/11ea5867-96eb-43df-b61f2cbe9673530d/policystatementsweetners.pdf
8. Markovic T P, Proietto J, Dixon J B, et al. The Australian Obesity Management Algorithm: A simple tool to guide the management of obesity in primary care. Obes Res Clin Pract 16, 353-363 (2022). doi:10.1016/j.orcp.2022.08.003
9. Ruanpeng, D., Thongprayoon, C., Cheungpasitporn, W. & Harindhanavudhi, T. Sugar and artificially sweetened beverages linked to obesity: a systematic review and meta-analysis. Qjm 110, 513-520 (2017).
10. Romo-Romo, A., Aguilar-Salinas, C. A., Brito-Córdova, G. X., Gómez-Diaz, R. A. & Almeda-Valdes, P. Sucralose decreases insulin sensitivity in healthy subjects: a randomized controlled trial. Am J Clin Nutr 108, 485-491 (2018).
11. Imamura, F., et al. Consumption of sugar sweetened beverages, artificially sweetened beverages, and fruit juice and incidence of type 2 diabetes: systematic review, meta-analysis, and estimation of population attributable fraction. BMJ: British Medical Journal 351, h3576 (2015).
12. Mossavar-Rahmani, Y., et al. Artificially Sweetened Beverages and Stroke, Coronary Heart Disease, and All-Cause Mortality in the Women's Health Initiative. Stroke 50, 555-562 (2019).
13. Malik, V. S., et al. Long-Term Consumption of Sugar-Sweetened and Artificially Sweetened Beverages and Risk of Mortality in US Adults. Circulation 139, 2113-2125 (2019).
14. Mullee, A., et al. Association Between Soft Drink Consumption and Mortality in 10 European Countries. JAMA Internal Medicine 179, 1479-1490 (2019).
15. Lohner, S., Toews, I. & Meerpohl, J. J. Health outcomes of non-nutritive sweeteners: analysis of the research landscape. Nutr J 16, 55 (2017).
16. Ron Perko, P. D. Sweeteners and Sugar Alternatives in Food Technology. in Sweeteners and Sugar Alternatives in Food Technology (ed. Mitchell, H.) 151-175 (Blackwell Publishing, 2006).
17. European Food Safety Authority. Statement in relation to the safety of erythritol (E 968) in light of new data, including a new paediatric study on the gastrointestinal tolerability of erythritol. EFSA Journal 8, 1650 (2010).
18. Food and Drug Administration. GRAS Notice (GRN) No. 789. (2018). Available at: https://www.fda.gov/media/132946/download
19. Bornet, F. R., Blayo, A., Dauchy, F. & Slama, G. Plasma and urine kinetics of erythritol after oral ingestion by healthy humans. Regul Toxicol Pharmacol 24, S280-285 (1996).
20. Hootman, K. C., et al. Erythritol is a pentose-phosphate pathway metabolite and associated with adiposity gain in young adults. Proc Natl Acad Sci USA 114, E4233-e4240 (2017).
21. Global Erythritol Market Research Report 2020. Available at: https://www.360researchreports.com/global-erythritol-market-15041957 (2020).
22. Yokozawa, T., Kim, H. Y. & Cho, E. J. Erythritol attenuates the diabetic oxidative stress through modulating glucose metabolism and lipid peroxidation in streptozotocin-induced diabetic rats. J Agric Food Chem 50, 5485-5489 (2002).
23. Flint, N., et al. Effects of erythritol on endothelial function in patients with type 2 diabetes mellitus: a pilot study. Acta Diabetol 51, 513-516 (2014).
24. Rebholz, C. M., et al. Serum metabolomic profile of incident diabetes. Diabetologia 61, 1046-1054 (2018).
25. Selvin, E., et al. Association of 1,5-Anhydroglucitol With Cardiovascular Disease and Mortality. Diabetes 65, 201-208 (2016).
26. Zhu, W., et al. Gut Microbial Metabolite TMAO Enhances Platelet Hyperreactivity and Thrombosis Risk. Cell 165, 111-124 (2016).
27. Schlicker, L., et al. Unexpected roles for ADH1 and SORD in catalyzing the final step of erythritol biosynthesis. J Biol Chem 294, 16095-16108 (2019).
28. Regnat, K., Mach, R. L. & Mach-Aigner, A. R. Erythritol as sweetener-wherefrom and whereto? Appl Microbiol Biotechnol 102, 587-595 (2018).
29. Tetzloff, W., Dauchy, F., Medimagh, S., Carr, D. & Bar, A. Tolerance to subchronic, high-dose ingestion of erythritol in human volunteers. Regul Toxicol Pharmacol 24, S286-295 (1996).
30. Bornet, F. R., Blayo, A., Dauchy, F. & Slama, G. Gastrointestinal response and plasma and urine determinations in human subjects given erythritol. Regul Toxicol Pharmacol 24, S296-302 (1996).
31. Munro, I. C., et al. Erythritol: an interpretive summary of biochemical, metabolic, toxicological and clinical data. Food Chem Toxicol 36, 1139-1174 (1998).
32. Gardener, H., et al. Diet soft drink consumption is associated with an increased risk of vascular events in the Northern Manhattan Study. J Gen Intern Med 27, 1120-1126 (2012).
33. Narain, A., Kwok, C. S. & Mamas, M. A. Soft drinks and sweetened beverages and the risk of cardiovascular disease and mortality: a systematic review and meta-analysis. Int J Clin Pract 70, 791-805 (2016).
34. Vyas, A., et al. Diet drink consumption and the risk of cardiovascular events: a report from the Women's Health Initiative. J Gen Intern Med 30, 462-468 (2015).
35. Lin, J. & Curhan, G. C. Associations of sugar and artificially sweetened soda with albuminuria and kidney function decline in women. Clin J Am Soc Nephrol 6, 160-166 (2011).
36. de Koning, L., et al. Sweetened beverage consumption, incident coronary heart disease, and biomarkers of risk in men. Circulation 125, 1735-1741, s1731 (2012).
37. de Koning, L., Malik, V. S., Rimm, E. B., Willett, W. C. & Hu, F. B. Sugar-sweetened and artificially sweetened beverage consumption and risk of type 2 diabetes in men. The American Journal of Clinical Nutrition 93, 1321-1327 (2011).
38. Suez, J., et al. Personalized microbiome-driven effects of non-nutritive sweeteners on human glucose tolerance. Cell 185, 3307-3328.e3319 (2022).
39. Debras, C., et al. Artificial sweeteners and risk of cardiovascular diseases: results from the prospective NutriNet-Santé cohort. BMJ 378, e071204 (2022).
40. Toews, I., Lohner, S., Küllenberg de Gaudry, D., Sommer, H. & Meerpohl, J. J. Association between intake of non-sugar sweeteners and health outcomes: systematic review and meta-analyses of randomised and non-randomised controlled trials and observational studies. Bmj 364, k4718 (2019).
41. Azad, M. B., et al. Nonnutritive sweeteners and cardiometabolic health: a systematic review and meta-analysis of randomized controlled trials and prospective cohort studies. Cmaj 189, E929-e939 (2017).
42. Miller, P. E. & Perez, V. Low-calorie sweeteners and body weight and composition: a meta-analysis of randomized controlled trials and prospective cohort studies. Am J Clin Nutr 100, 765-777 (2014).
43. McGlynn, N. D., et al. Association of Low- and No-Calorie Sweetened Beverages as a Replacement for Sugar-Sweetened Beverages With Body Weight and Cardiometabolic Risk: A Systematic Review and Meta-analysis. JAMA Netw Open 5, e222092 (2022).
44. Sylvetsky, A. C., Blau, J. E. & Rother, K. I. Understanding the metabolic and health effects of low-calorie sweeteners: methodological considerations and implications for future research. Rev Endocr Metab Disord 17, 187-194 (2016).
45. Wang, Z., et al. Metabolomic Pattern Predicts Incident Coronary Heart Disease. Arterioscler Thromb Vase Biol 39, 1475-1482 (2019).

### Methods-Only References

46. Tang, W. H., et al. Intestinal microbial metabolism of phosphatidylcholine and cardiovascular risk. N Engl J Med 368, 1575-1584 (2013).
47. Wang, Z., et al. Gut flora metabolism of phosphatidylcholine promotes cardiovascular disease. Nature 472, 57-63 (2011).
48. Stevens, L. A., et al. Comparative performance of the CKD Epidemiology Collaboration (CKD-EPI) and the Modification of Diet in Renal Disease (MDRD) Study equations for estimating GFR levels above 60 mL/min/1.73 m2. Am J Kidney Dis 56, 486-495 (2010).
49. König, M., et al. Cohort profile: role of lipoproteins in cardiovascular disease—the LipidCardio study. BMJ Open 9, e030097 (2019).
50. STROBE Statement—checklist of items that should be included in reports of observational studies1 (© STROBE Initiative). International Journal of Public Health 53, 3-4 (2008).
51. Nemet, I., et al. A Cardiovascular Disease-Linked Gut Microbial Metabolite Acts via Adrenergic Receptors. Cell 180, 862-877.e822 (2020).
52. Gupta, N., Li, W. & McIntyre, T. M. Deubiquitinases Modulate Platelet Proteome Ubiquitination, Aggregation, and Thrombosis. Arterioscler Thromb Vasc Biol 35, 2657-2666 (2015).
53. Scavone, M., et al. Platelet Adhesion and Thrombus Formation in Microchannels: The Effect of Assay-Dependent Variables. Int J Mol Sci 21, 750 (2020).
54. Witkowski, M., et al. Vascular endothelial tissue factor contributes to trimethylamine N-oxide-enhanced arterial thrombosis. Cardiovascular Research (2021).
55. Fiehn, O., Wohlgemuth, G. & Scholz, M. Setup and Annotation of Metabolomic Experiments by Integrating Biological and Mass Spectrometric Metadata. in Data Integration in the Life Sciences (eds. Luduscher, B. & Raschid, L.) 224-239 (Springer Berlin Heidelberg, Berlin, Heidelberg, 2005).
56. Wilson, P. W., et al. Prediction of coronary heart disease using risk factor categories. Circulation 97, 1837-1847 (1998).
57. SCORE2 working group and ESC Cardiovascular risk collaboration. SCORE2 risk prediction algorithms: new models to estimate 10-year risk of cardiovascular disease in Europe. European Heart Journal 42, 2439-2454 (2021).

### Example 3

### Ingestion of the Artificial Sweetener Erythritol Enhances Platelet Reactivity and Thrombosis Potential in Healthy Volunteers

Artificial sweeteners (including non-nutritive sweeteners) are widely used and generally regarded as safe (GRAS). However, a growing number of epidemiological studies have associated their use with cardiovascular disease (CVD) risk.(1). Epidemiological studies are limited by potential unmodeled confounding, including reverse causation. Further, due to current limitations in food labelling requirements, epidemiology studies generally do not quantify individual sweeteners, but instead use food questionnaires to estimate consumption in broad categories (e.g. “natural sweeteners”, or “sugar alcohols”). While numerous international health bodies (e.g. ADA, AHA, EFSA) recommend use of artificial sweeteners over sugar to patients most at risk for thrombotic events (e.g. those with diabetes, obesity, metabolic syndrome),(2) human intervention studies to directly assess adverse effects of sweeteners are limited.

We reported in Example 2 above fasting plasma levels of the widely used non-nutritive sweetener erythritol are clinically associated with incident major adverse cardiovascular event (MACE) risks in both US and EU cohorts.(3) We also showed elevated circulating erythritol levels enhanced thrombosis potential in animal models, and elicited pro-thrombotic phenotypic changes to platelets in multiple in vitro studies.(3). Moreover, consumption of erythritol lead to prolonged (days) heightened levels in subjects, further raising safety concerns. (3) In this Example, using a prospective interventional study design with a relevant dietary exposure in healthy volunteers, we assessed the pro-thrombotic effects of dietary erythritol (E 968), one of the fastest growing Food and Drug Administration (FDA) and European Food Safety Authority (EFSA) approved “zero-calorie” or non-nutritive sweeteners.(4).

This single center trial was approved by the Cleveland Clinic Institutional Review Board (clinicaltrials.gov identifier NCT04731363). Following subject consent (nonsmokers without CVD, hypertension, or diabetes), blood was drawn after overnight fast and 30 min following consumption of water containing 30 g of ether glucose (n=10, 30.1±11 years of age, 40% male), or erythritol (n=10, 30.5±8 years of age, 50% male), a quantity commonly found in erythritol-sweetened foods and the daily intake of some subjects based on 2013-2014 National Health and Nutrition Examination Survey (NHANES) data and FDA filings.(4,5).

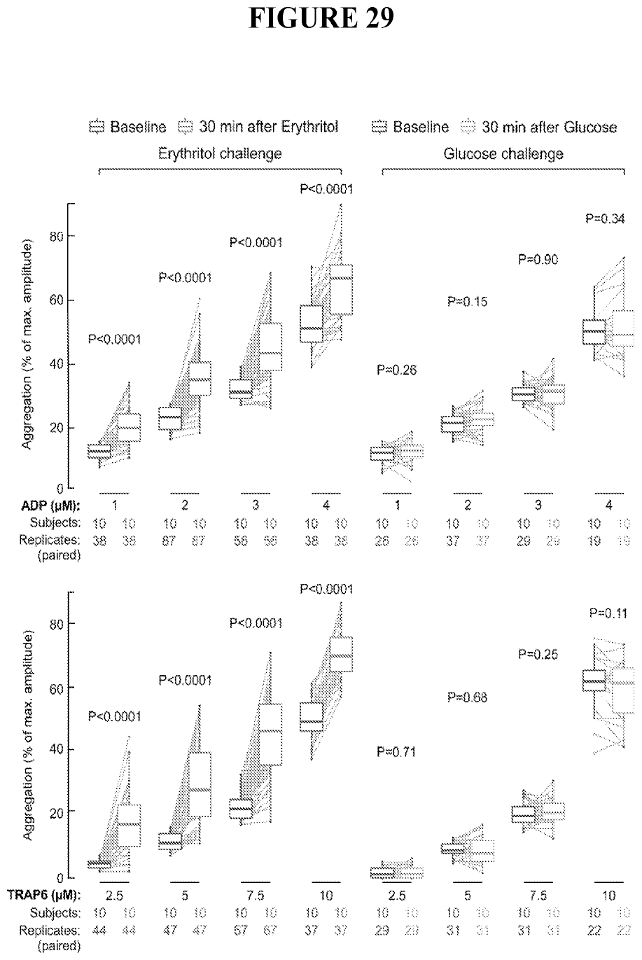

Following erythritol consumption, postprandial circulating erythritol levels (assessed using stable-isotope-dilution liquid-chromatography tandem mass spectrometry (LC/MS/MS)3) were >1000-fold increased compared to baseline levels (median[inter quartile range], 6480[5930-7300]μM versus 3.75[3.35-3.87]μM, P<0.0001). In contrast, circulating erythritol levels remained similar before versus after glucose consumption (3.0[2.6-4.0]μM versus 2.9[2.7-3.8]μM, P=0.87; glucose levels before versus after 86.5(81.8-93.0) versus 127.0(122.3-132.3), P=0.002). A striking increase in platelet aggregation responses to both ADP and TRAP6 were observed in all subjects following ingestion of erythritol (FIG. 29, P<0.0001 for all subjects, agonists and doses examined). In contrast, glucose consumption had no effect on platelet aggregation. The erythritol-dependent increase in platelet responsiveness showed significant correlation among all subjects (erythritol levels versus agonist-induced aggregation; Spearman rho 0.65 and 0.68, P<0.0001 each, for ADP and TRAP6, respectively). Erythritol ingestion also markedly enhanced stimulus-dependent release of both the dense granule marker serotonin (P<0.001 for both TRAP6 and ADP; quantified by LC/MS/MS), and the alpha granule marker CXCL4 (P<0.001 for TRAP6 (7.5 μM); P=0.06 for ADP (2.0 μM); quantified by ELISA, R&D Systems, MN, USA). In contrast, no differences were observed in stimulus (ADP, TRAP6) dependent release of either serotonin or CXCL4 following glucose ingestion.

The present example shows that a standard serving of erythritol elicits a direct pro-thrombotic effect in healthy subjects with normal renal function. The present findings strongly advise for long-term safety studies of artificial sweeteners generally, and erythritol specifically. They also suggest guideline recommendations of erythritol as a GRAS supplement need to be reassessed. This is particularly relevant to consumers to be able to make informed decisions on erythritol amounts added to processed foods.

### References for Example 3

1. Malik V S, Li Y, Pan A, De Koning L, Schernhammer E, Willett W C and Hu F B. Long-Term Consumption of Sugar-Sweetened and Artificially Sweetened Beverages and Risk of Mortality in US Adults. Circulation. 2019; 139:2113-2125.
2. Gardner C, Wylie-Rosett J, Gidding S S, Steffen L M, Johnson R K, Reader D and Lichtenstein A H. Nonnutritive sweeteners: current use and health perspectives: a scientific statement from the American Heart Association and the American Diabetes Association. Circulation. 2012; 126:509-19.
3. Witkowski M, Nemet I, Alamri H, Wilcox J, Gupta N, Nimer N, Haghikia A, Li X S, Wu Y, Saha P P, Demuth I, K6nig M, Steinhagen-Thiessen E, Cajka T, Fiehn O, Landmesser U, Tang W H W, and Hazen S L. The Artificial Sweetener Erythritol and Cardiovascular Event Risk. Nat Med. 2023 Feb. 27. doi: 10.1038/s41591-023-02223-9. Online ahead of print.
4. Food and Drug Administration. GRAS Notice (GRN) No. 789. (2018). Available at: https://www.fda.gov/media/132946/download
5. European Food Safety Authority. Statement in relation to the safety of erythritol (E 968) in light of new data, including a new paediatric study on the gastrointestinal tolerability of erythritol. EFSA Journal 8, 1650 (2010).

Although only a number exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

We claim:

1. A method of detecting the level of xylitol in a biological sample that may also contain xylitol's structural isomers arabitol and rabitol, comprising:
a) mixing a biological sample from a subject with at least one stable isotope labeled structural isomer (SIL isomer) family member selected from: stable isotope labelled xylitol (SIL-xylitol), SIL-arabitol, and SIL-rabitol,
b) mixing said biological sample with a hydroxyl group modifying agent such that said SIL isomer and xylitol and any arabitol and rabitol present in said biological sample are modified, thereby generating modified-xylitol and modified SIL isomer, and generating modified-arabitol and modified-rabitol if present;
c) subjecting said biological sample to purification such that said modified-xylitol and modified SIL isomer, and modified-arabitol modified-rabitol is present, are substantially isolated from other components of said biological sample, thereby generating a purified sample;
d) subjecting said purified sample to mass spectrometry such that peak intensity values are generated for said modified-xylitol and said modified SIL isomer, and said modified-arabitol and modified-rabitol if present; and
e) employing the peak intensity value of said modified SIL isomer as an internal standard, and the peak intensity of said modified-xylitol, to determine the level of said xylitol present in said biological sample.

2. The method of claim 1, wherein said subject has, or is suspected of having, cardiovascular disease, or has an increased risk of: developing cardiovascular disease, having a major adverse cardiovascular event, risk of stroke, or having enhanced thrombosis.

3. The method of claim 1, wherein said biological sample is selected from a urine sample, plasma sample, blood sample, serum sample, sputum, or liquified stool sample.

4. The method of claim 1, wherein said hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.

5. The method of claim 1, wherein said modified-xylitol, modified SIL isomer, modified-arabitol, modified-rabitol are esterified or acetylated versions of said xylitol, SIL isomer, arabitol, and rabitol.

6. The method of claim 1, wherein said subject reports a higher than average intake of xylitol in their diet.

7. The method of claim 1, wherein said at least one stable isotope labeled structural isomer comprises stable isotope labelled arabitol (SIL-arabitol).

8. The method of claim 1, wherein said at least one stable isotope labeled structural isomer comprises stable isotope labelled xylitol (SIL-xylitol).

9. The method of claim 1, wherein said at least one stable isotope labeled structural isomer comprises stable isotope labelled rabitol (SIL-rabitol).

10. The method of claim 1, further comprising: generating a report comprising said level of xylitol, wherein said report identifies said level of xylitol as higher than a control level.

11. The method of claim 10, wherein said report describes that said subject should reduce intake of xylitol in order to improve: risk of cardiac disease and/risk of thrombosis.

12. The method of claim 10, wherein said report describes that said subject should be prescribed and/or administered at least one of the following: a CVD therapeutic, an anti-coagulating agent, an anti-platelet agent, a lipid lowering agent, and a blood pressure control agent or therapy.

13. The method of claim 1, wherein said mass spectrometry comprises MS/MS, and/or wherein said stable isotope is selected from C13 and deuterium.

14. The method of claim 1, wherein said subject is a human.

15. A method of detecting the level of erythritol in a biological sample that also contains erythritol's structural isomer threitol, comprising:
a) mixing a biological sample from a subject with at least one stable isotope labeled structural isomer (SIL isomer) family member selected from: stable isotope labelled erythritol (SIL-erythritol) and SIL-threitol,
b) mixing said biological sample with a hydroxyl group modifying agent such that said SIL isomer and erythritol and any threitol present in said biological sample are modified, thereby generating modified-erythritol, modified-threitol, and modified SIL isomer;
c) subjecting said biological sample to purification such that said modified-erythritol, modified-threitol, and said modified SIL isomer are substantially isolated from other components of said biological sample, thereby generating a purified sample; and
d) subjecting said purified sample to mass spectrometry such that peak intensity values are generated for said modified-erythritol, modified-threitol, and said modified SIL isomer; and
e) employing the peak intensity value of said modified SIL isomer as an internal standard, and the peak intensity of said modified-erythritol, to determine the level of said erythritol present in said biological sample.

16. The method of claim 15, wherein said subject has, or is suspected of having, cardiovascular disease, or has an increased risk of: developing cardiovascular disease, having a major adverse cardiovascular event, risk of stroke, or having enhanced thrombosis.

17. The method of claim 15, wherein said biological sample is selected from a urine sample, plasma sample, blood sample, serum sample, sputum, or liquified stool sample.

18. The method of claim 15, wherein said hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.

19. The method of claim 15, wherein said modified-erythritol, modified SIL isomer, and modified-threitol are esterified or acetylated versions of said erythritol, SIL isomer, and threitol.

20. The method of claim 15, wherein said subject reports a higher than average intake of erythritol in their diet.

21. The method of claim 15, wherein said at least one stable isotope labeled structural isomer comprises stable isotope labelled erythritol (SIL-erythritol).

22. The method of claim 15, wherein said at least one stable isotope labeled structural isomer comprises stable isotope labelled threitol (SIL-threitol).

23. The method of claim 15, further comprising: generating a report comprising said level of erythritol, wherein said report identifies said level of erythritol as higher than a control level.

24. The method of claim 23, wherein said report describes that said subject should reduce intake of erythritol in order to improve: risk of cardiac disease and/risk of thrombosis.

25. The method of claim 23, wherein said report describes that said subject should be prescribed and/or administered at least one of the following: a CVD therapeutic, an anti-coagulating agent, an anti-platelet agent, a lipid lowering agent, and a blood pressure control agent or therapy.

26. The method of claim 15, wherein said mass spectrometry comprises MS/MS, and/or wherein said stable isotope is selected from C13 and deuterium.

27. The method of claim 15, wherein said subject is a human.

28. A method comprising:
a) receiving results of, or conducting, a circulating polyol sweetener analysis on a biological sample from a subject with, or suspected of having, or at increased risk of developing: a cardiovascular disease, a major adverse cardiovascular event, or enhanced thrombosis; and
b) performing at least one of the following after identifying said biological sample as having higher levels of said polyol sweetener compared to control levels,
i) treating said subject with a CVD therapeutic, anti-coagulating agent, anti-platelet agent;
ii) treating said subject with lipid lowering agents, or BP control agent or therapy;
iii) treating said subject with antiplatelet agents, due to the increased thrombosis risks associated with elevated polyol sweetener,
iv) prescribing, or administering, to said subject a dietary intervention to reduce polyol sweetener levels in their diet;
v) treating said subject with heart failure related therapy efforts; and/or
vi) transmitting a report to said subject or medical personnel treating said subject, indicating said subject is suitable for, or should be, treated with any of i)-v) above.

29. The method of claim 28, wherein said polyol sweetener is erythritol or xylitol.

30. The method of claim 28, wherein said polyol sweetener is selected from: Isothreonic acid, threitol, pseudouridine, arabitol, myo-inositol, xylose, cellobiose, mannose, levoglucosan, saccharic acid, conduritol beta epoxide, lactulose, glycerol, 1,5-AHG, sucrose, beta-glycerol phosphate, glyceric acid, uridine, shikimic acid, 2-monoolein, threonic acid, fucose, UDP-glucuronic acid, fructose, ascorbate, maltose, inosine, glycerol-3-galactoside, propane-1,3-diol, glucose, 1,2-anhydro-myo-inositol, 1-monopalmitin, 6-deoxyyhexitol, 5-deoxyglucitol, glycerol-alpha-phosphate, 3-phosphoglycerate, 1-monstearin, 1-monolein, quinic acid, and diglycerol.

31. The method of claim 1, wherein said subject has at least one of the following: coronary artery disease (CAD), peripheral artery disease (PAD), cerebrovascular disease (CVD), transient ischemic attack (TIA), acute coronary syndrome (ACS), arterial aneurysm, heart failure (heart failure with preserved ejection fraction (HFpEF), heart failure with reduced ejection fraction (HFrEF)), need for revascularization (CABG, angioplasty, stent), or enhanced thrombosis, non-ST-segment myocardial infarction (NSTEMI), and ST-segment myocardial infarction (STEMI).

32. The method of claim 28, said prescribing, or administering, to said subject a dietary intervention to reduce polyol sweetener levels in their diet comprises recommending to said subject at least one of the following: avoidance of artificial sweeteners, avoidance of processed foods, reduction in high glycemic index related foods, caloric restriction diet, and a weight loss diet.

33. The method of claim 28, wherein said biological sample is selected from a urine sample, plasma sample, blood sample, serum sample, sputum, or liquified stool sample.

34. The method of claim 28, wherein said anti-platelet agents is selected from: ASA, also called acetylsalicylic acid (Aspirin, Asaphen, Entrophen, Novasen); Clopidogrel (Plavix); Prasugrel (Effient); and Ticagrelor (Brilinta).

35. The method of claim 28, wherein said anti-coagulant is selected from: a coumarin, a indandione, a factor Xa inhibitor, a heparin, a thrombin inhibitor, rivaroxaban (Xarelto), dabigatran (Pradaxa), apixaban (Eliquis), edoxaban (Lixiana), and Warfarin.

36. The method of claim 28, wherein said conducting a circulating polyol sweetener analysis comprises: i) mixing said biological sample from a subject with at least one stable isotope labeled structural isomer (SIL isomer) family member that includes said stable isotope labelled polyol sweetener and a first structural isomer of said polyol sweetener.

37. The method of claim 36, wherein said conducting a circulating polyol sweetener analysis further comprises: ii) mixing said biological sample with a hydroxyl group modifying agent agent such that said SIL isomer and said polyol sweetener are modified, thereby generating modified-polyol sweetener and modified SIL isomer.

38. The method of claim 37, wherein said conducting a circulating polyol sweetener analysis further comprises: iii) subjecting said biological sample to purification such that said modified-polyol sweetener and said modified SIL isomer are substantially isolated from other components of said biological sample, thereby generating a purified sample.

39. The method of claim 38, wherein said conducting a circulating polyol sweetener analysis further comprises: iv) subjecting said purified sample to mass spectrometry such that peak intensity values are generated for said modified-polyol sweetener and said modified SIL isomer, and optionally further comprises: comprises: v) employing the peak intensity value of said modified SIL isomer as an internal standard, and the peak intensity of said modified-polyol sweetener, to determine the level of said polyol sweetener present in said biological sample.

40. The method of claim 28, wherein said conducting a circulating polyol sweetener analysis comprises: i) the use of mass spectrometry of the analytes; ii) the use of immunological detection of the analytes; iii) the use of colorimetric detection of the analytes); iv) the use of electrochemical detection of the analytes; v) the use of fluorescence detection of the analytes, vi) the use of spectrophotometric detection of analytes; vii) the use of chemiluminescence detection of analytes; viii) the use of ambient ionization mass spectrometry to detect analytes.

41. The method of claim 28, wherein said report comprises a paper report or an electronic report; and/or wherein said receiving information comprises receiving said report, wherein said receiving said report is optionally via: 1) the mail system, 2) email, or 3) via a LAN of a hospital or clinic.

42. The method of claim 28, wherein said transmitting said report comprises: 1) mailing the reporting through the mail system, 2) emailing the report over the internet, or 3) sending the report through a local area network (LAN) or a hospital or clinic.

43. The method of claim 28, wherein said control value is derived from a sample from the general public or from a group known to not have cardiovascular disease or be at elevated risk for a thrombotic event.

44. The method of claim 28, wherein said conducting a circulating polyol sweetener analysis is performed with an analytical device selected from: a mass spectrometer, NMR spectrometer, and a UV/Vis spectrometer.

45. A kit or system or composition comprising:
a) at least one stable isotope labeled structural isomer (SIL isomer) family member selected from: stable isotope labelled xylitol (SIL-xylitol), SIL-arabitol, and SIL-rabitol; and
b) at least one of the following:
i) a mass spectrometer,
ii) a hydroxyl group modifying agent;
iii) a biological sample from a subject, optionally wherein the subject with, or suspected of having, or at increased risk of developing: a cardiovascular disease, a major adverse cardiovascular event, or enhanced thrombosis.

46. The kit or system of claim 45, wherein said mass spectrometer is present.

47. The kit, system, or composition of claim 45, wherein said hydroxyl group modifying agent and/or biological sample is present.

48. The kit, system, or composition of claim 47, wherein said hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.

50. A kit or system or composition comprising:
a) at least one stable isotope labeled structural isomer (SIL isomer) family member selected from: stable isotope labelled erythritol (SIL-erythritol) and SIL-threitol; and
b) at least one of the following:
i) a mass spectrometer,
ii) hydroxyl group modifying agent;
iii) a biological sample from a subject, optionally wherein the subject with, or suspected of having, or at increased risk of developing: a cardiovascular disease, a major adverse cardiovascular event, or enhanced thrombosis.

51. The kit or system of claim 50, wherein said mass spectrometer is present.

52. The kit, system, or composition of claim 50, wherein said hydroxyl group modifying agent and/or biological sample is present.

53. The kit, system, or composition of claim 50, wherein said hydroxyl group modifying agent is selected from: acetic anhydride, an organic anhydride, an acyl chloride, an isocyanate, a silation agents, MTBSTFA, BSTFA, N,O-bis(trimethylsily)trifluoroacetamide (BSTFA), N-methyl-N(trimethylsily) trifluoroacetamide (MSTFA), an alkyldimethylsilyl agents, acetic anhydride, propionic anhydride, butyric anhydride, pentafluorobenzoyl anhydride, and heptafluorobutyric acid anhydride.
